SEQUENCE LISTING (11 sequences)

SEQ ID NO: 1 - protein, 450 aa, synthetic construct
  REGION 1..450: note = Heavy chain of humanized anti-HER2 antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
  REGION 1..214: note = Light chain of humanized anti-HER2 antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 8 aa, synthetic construct
  REGION 1..8: note = CDRH1 of heavy chain of humanized anti-HER2 antibody
GFNIKDTY

SEQ ID NO: 4 - protein, 8 aa, synthetic construct
  REGION 1..8: note = CDRH2 of heavy chain of humanized anti-HER2 antibody
IYPTNGYT

SEQ ID NO: 5 - protein, 13 aa, synthetic construct
  REGION 1..13: note = CDRH3 of heavy chain of humanized anti-HER2 antibody
SRWGGDGFYAMDY

SEQ ID NO: 6 - protein, 6 aa, synthetic construct
  REGION 1..6: note = CDRL1 of light chain of humanized anti-HER2 antibody
QDVNTA

SEQ ID NO: 7 - protein, 7 aa, synthetic construct
  REGION 1..7: note = Sequence comprising CDRL2 of light chain of humanized anti-HER2 antibody
SASFLYS

SEQ ID NO: 8 - protein, 9 aa, synthetic construct
  REGION 1..9: note = CDRL3 of light chain of humanized anti-HER2 antibody
QQHYTTPPT

SEQ ID NO: 9 - protein, 120 aa, synthetic construct [sequence omitted]

SEQ ID NO: 10 - protein, 107 aa, synthetic construct [sequence omitted]

SEQ ID NO: 11 - protein, 449 aa, synthetic construct
  REGION 1..449: note = Heavy chain of humanized anti-HER2 antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2025/0161305 A1
(43) Pub. Date: May 22, 2025

(54) COMBINATION OF ANTIBODY-DRUG CONJUGATE AND RASG12C INHIBITOR

(71) Applicants: AstraZeneca UK Limited, Cambridge (GB); DAIICHI SANKYO COMPANY, LIMITED, Tokyo (JP)

(72) Inventors: Sarah Jane ROSS, Cambridge (GB); Jerome Thomas METTETAL, II, Wilmington, DE (US); Atanu CHAKRABORTY, Cambridge (GB)

(73) Assignees: AstraZeneca UK Limited, Cambridge (GB); DAIICHI SANKYO COMPANY, LIMITED, Tokyo (JP)

(21) Appl. No.: 18/724,593

(22) PCT Filed: Dec. 27, 2022
(86) PCT No.: PCT/IB2022/062797

§ 371 Date: Jun. 26, 2024

Related U.S. Application Data
(60) Provisional application No. 63/294,367, filed on Dec. 28, 2021.

(51) Int. Cl. A61K 31/519; A61K 47/68; A61P 35/00

(52) U.S. Cl. CPC A61K 31/519; A61K 47/68037; A61K 47/6851; A61P 35/00

(57) ABSTRACT

A pharmaceutical product for administration of an anti HER2 antibody-drug conjugate in combination with a RASG12C inhibitor is provided. Also provided is a therapeutic use and method wherein the antibody-drug conjugate and the RASG12C inhibitor are administered in combination to a subject.

### TECHNICAL FIELD

The present disclosure relates to a pharmaceutical product for administration of an antibody-drug conjugate, having an antitumor drug conjugated to an anti-HER2 antibody via a linker structure, in combination with a RASG12C inhibitor, and to a therapeutic use and method wherein the specific antibody-drug conjugate and the RASG12C inhibitor are administered in combination to a subject.

### BACKGROUND

The KRAS, NRAS and HRAS genes encode a set of closely related small GTPase proteins KRas, NRas and HRas, collectively referred to herein as the Ras proteins or Ras, that share 82-90% overall sequence identity. The Ras proteins are critical components of signalling pathways transmitting signals from cell-surface receptors to regulate cellular proliferation, survival and differentiation.

The Ras proteins are 188-189 amino acids in length and function as molecular switches, cycling between an inactive GDP-bound state and an active GTP-bound state. In particular, the Ras proteins have a highly conserved N-terminal G-domain containing the p-loop region, which binds nucleotide, and the switch I and switch II regions which are important for regulatory and effector protein interactions. The best characterised effector of Ras is the serine/threonine kinase Raf which regulates the activity of the mitogen-activate protein kinase (MAPK) pathway. The PI3K pathway is another important effector pathway down-stream of Ras with the p110 catalytic subunit of the class I phosphoinositide 3-kinases interacting with Ras. Other effectors of Ras including RalGDS, Tiam1, PLC-e and Rassf1 have been have also been described (Cox, et al. Nature Reviews Drug Discovery, 2014, 13:828-851).

RAS mutations are frequently found in cancer and approximately 30% of all human cancers have a mutation in KRAS, NRAS or HRAS genes. Oncogenic Ras is typically, but not exclusively, associated with mutations at glycine 12, glycine 13 or glutamine 61 of Ras. These residues are located at the active site of Ras and mutations impair intrinsic and/or GAP-catalysed GTPase activity favouring the formation of GTP bound Ras and aberrant activation of down-stream effector pathways. KRAS is the most frequently mutated RAS gene in cancer followed by NRAS and then HRAS. There are several tumour types that exhibit a high frequency of activating mutations in KRAS including pancreatic (˜90% prevalence), colorectal (˜40% prevalence) and non-small cell lung cancer (˜30% prevalence). KRAS mutations are also found in other cancer types including multiple myeloma, uterine cancer, bile duct cancer, stomach cancer, bladder cancer, diffuse large B cell lymphoma, rhabdomyosarcoma, cutaneous squamous cell carcinoma, cervical cancer, testicular germ cell cancer and others.

Glycine to cysteine mutations at residue 12 of Ras (the G12C mutation) is generated from a G.C to T.A base transversion at codon 12, a mutation commonly found in RAS genes that accounts for 14% of all KRAS, 2% of all NRAS and 2% of all HRAS mutations across cancer types. The G12C mutation is particularly enriched in KRAS mutant non-small cell lung cancer with approximately half carrying this mutation, which has been associated with the DNA adducts formed by tobacco smoke. The G12C mutation is not exclusively associated with lung cancer and is found in other RAS mutant cancer types including 8% of all KRAS mutant colorectal cancer.

Compounds exhibiting G12C mutant Ras proteins inhibitory activity (RASG12C inhibitors) are disclosed, for example, in WO2019/099524, WO2019/215203, WO2020/178282, WO2021/118877, WO2021/245051. Known RASG12C inhibitors include compounds designated as LY3537982 (Loxo/Lilly), AZD4625 (AstraZeneca), AMG510 (sotorasib: Amgen), MRTX849 (adagrasib: Mirati), JDQ443 (Novartis), GDC-6036 (Genentech), BI 1,823,911 (Boehringer Ingelheim), D1553 (InventisBio) and JNJ-74699157 (Johnson and Johnson).

Antibody-drug conjugates (ADCs), which are composed of a cytotoxic drug conjugated to an antibody, can deliver the drug selectively to cancer cells, and are therefore expected to cause accumulation of the drug within cancer cells and to kill the cancer cells (Ducry, L., et al., Bioconjugate Chem. (2010) 21, 5-13; Alley, S. C., et al., Current Opinion in Chemical Biology (2010) 14, 529-537; Damle N. K. Expert Opin. Biol. Ther. (2004) 4, 1445-1452; Senter P. D., et al., Nature Biotechnology (2012) 30, 631-637; Burris HA., et al., J. Clin. Oncol. (2011) 29(4): 398-405).

ADCs comprising an anti-HER2 antibody conjugated to a drug via a linker are disclosed, for example, in WO2015/115091. Known ADCs include trastuzumab emtansine (Kadcyla®, T-DM1), which is composed of a HER2-targeting antibody (trastuzumab) covalently linked to a cytotoxic microtubule inhibitor (DM1), and trastuzumab deruxtecan (Enhertu®, DS-8201), which is composed of trastuzumab and a derivative of exatecan (Ogitani Y. et al., Clinical Cancer Research (2016) 22(20), 5097-5108; Ogitani Y. et al., Cancer Science (2016) 107, 1039-1046).

Trastuzumab deruxtecan (Enhertu®, DS-8201) has shown significant clinical efficacy in HER2-expressing solid tumors, including breast cancer, gastric cancer, colorectal cancer and non-small cell lung cancer. Significantly, DS-8201 has demonstrated promising activity in HER2 low tumors in the above indications. However, there is still a need to identify combination partners for DS-8201 to enhance its therapeutic potential.

Despite the therapeutic potential of anti-HER2 ADCs such as trastuzumab emtansine and trastuzumab deruxtecan, and of RASG12C inhibitors, a need remains for improved therapeutic compositions and methods, that can enhance efficacy of existing cancer treating agents, increase durability of therapeutic response, improve tolerance to patients and/or reduce dose-dependent toxicity.

### SUMMARY OF DISCLOSURE

Various antibody-drug conjugates, such an anti-HER2 antibody-drug conjugate that includes a microtubule inhibitor (DM1) or a derivative of the topoisomerase I inhibitor exatecan, as a component, have been shown to exhibit an excellent antitumor effect in the treatment of certain cancers such as breast cancer and gastric cancer, when administered singly. Furthermore, RASG12C inhibitors are known that have been confirmed to exhibit an antitumor effect in the treatment of certain cancers. However, it is desired to provide a medicine and treatment which can obtain a superior antitumor effect in the treatment of cancers, such as enhanced efficacy, increased durability of therapeutic response and/or reduced dose-dependent toxicity.

The present disclosure provides a pharmaceutical product which can exhibit an excellent antitumor effect in the treatment of cancers, through administration of an anti-HER2 antibody-drug conjugate in combination with a RASG12C inhibitor. The present disclosure also provides a therapeutic use and method wherein the anti-HER2 antibody-drug conjugate and RASG12C inhibitor are administered in combination to a subject.

Specifically, the present disclosure relates to the following [1] to [63]:

[1] a pharmaceutical product comprising an antibody-drug conjugate in which the antibody is an anti-HER2 antibody, and a RASG12C inhibitor for administration in combination.
[2] the pharmaceutical product according to [1], wherein the RASG12C inhibitor is a compound represented by the following formula (I):

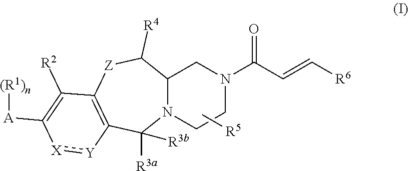

wherein:
A is phenyl or a bicyclic heteroaryl group;
X and Y are connected by a double bond and i) X is CR⁷ and Y is CR⁸, ii) X is N and Y is CR⁸, or iii) X is CR⁷ and Y is N; or
X and Y together are C(O)NR⁹; or
X and Y are adjacent ring atoms of an optionally substituted 5- or 6-membered N-heterocycle fused to the aromatic ring substituted with Z, and X and Y are both C or are C and N;
Z is O, NH, or NMe;
R¹ is independently selected from F, Cl, Br, OH, CH₂OH, OMe, CH₂OMe, C₁-C₃alkyl and C₁-C₃fluoroalkyl;
n is 0, 1, 2 or 3;
R² is H, F, Cl, CCH, CCMe, CN, Br, C₁-C₃alkyl, C₁-C₃fluoroalkyl, OMe or OEt;
R^{3a} and R^{3b} together are ═O or R^{3a} and R^{3b} are H;
R⁴ is H or Me;
R⁵ is H or Me;
R⁶ is H or CH₂NMe₂;
R⁷ and R⁸ are independently selected from H, F, Cl, CCH, CC(C₁-C₃alkyl), CCCH₂NMe₂, CCCH₂O(C₁-C₃alkyl), CN, Me, C₁-C₆alkyl, OH, OMe, O(C₁-C₃alkyl), O(C₁-C₃deuteroalkyl), O(C₁-C₃fluoroalkyl), O(C₃-C₆cycloalkyl), C₁-C₃fluoroalkyl, OCH₂CH₂NMe₂, OCH₂CH₂OMe, CH₂OMe, OCH₂CH₂N(CH₂CH₂)₂CH, OCH₂CH₂N(CH₂CH₂) 20, OCH₂CH₂ (2-pyridyl) or an optionally substituted 3-, 4-, 5- or 6-membered carbocycle or heterocycle; or
R⁷ and R⁸ combine to form an optionally substituted 5- or 6-membered carbocycle or heterocycle;
R⁹ is selected from H, Me, Et, C₃H₇ and C₁-C₃fluoroalkyl; or a pharmaceutically acceptable salt thereof;
   [3] the pharmaceutical product according to [2] wherein, in formula (I), i) X is CR⁷ and Y is CR⁸, ii) X is N and Y is CR⁸ or iii) X is CR⁷ and Y is N;
   [4] the pharmaceutical product according to [2] or [3] wherein, in formula (I), Z is O;
   [5] the pharmaceutical product according to any one of [2] to [4] wherein, in formula (I), R^{3a} and R^{3b} are H;
   [6] the pharmaceutical product according to any one of [2] to [5] wherein, in formula (I), R⁴ is H;
   [7] the pharmaceutical product according to any one of [2] to [6] wherein, in formula (I), R⁶ is H;
   [8] the pharmaceutical product according to any one of [2] to [7] wherein, in formula (I), A is phenyl;
   [9] the pharmaceutical product according to [2], wherein the RASG12C inhibitor is a compound selected from:
(12aS)-2-Acryloyl-10-chloro-9-(5-methyl-1H-indazol-4-yl)-1,2,3,4,12,12a-hexahydro-6H-benzo[f]pyrazino[2,1-c][1,4]oxazepin-6-one;
1-((12aS)-10-Chloro-9-(5-methyl-1H-indazol-4-yl)-3,4,12,12a-tetrahydro-6H-benzo[f]pyrazino[2,1-c][1,4]oxazepin-2(1H)-yl)prop-2-en-1-one;
1-[(12aR)-10-Chloro-9-(2-fluoro-6-hydroxyphenyl)-7-methoxy-3,4,12,12a-tetrahydro-6H-pyrazino[2,1-c][1,4]benzoxazepin-2(1H)-yl]prop-2-en-1-one;
(12aR)-10-chloro-9-(2-fluoro-6-hydroxyphenyl)-7-hydroxy-2-(prop-2-enoyl)-1,2,3,4,12,12a-hexahydro-6H-pyrazino[2,1-c][1,4]benzoxazepin-6-one;
(12aR)-10-Chloro-9-(5-methyl-1H-indazol-4-yl)-2-(prop-2-enoyl)-1,2,3,4,12,12a-hexahydro-6H-pyrazino[2,1-c][1,4]benzoxazepin-6-one;
1-((12aR)-10-Chloro-9-(5-methyl-1H-indazol-4-yl)-3,4,12,12a-tetrahydro-6H-benzo[f]pyrazino[2,1-c][1,4]oxazepin-2(1H)-yl)prop-2-en-1-one;
(12aR)-10-Chloro-8-fluoro-9-(2-fluoro-6-hydroxyphenyl)-2-(prop-2-enoyl)-1,2,3,4,12,12a-hexahydro-6H-pyrazino[2,1-c][1,4]benzoxazepin-6-one;
1-[(12aR)-8,10-dichloro-9-(2-fluoro-6-hydroxyphenyl)-7-hydroxy-3,4,12,12a-tetrahydro-6H-pyrazino[2,1-c][1,4]benzoxazepin-2(1H)-yl]prop-2-en-1-one;
1-[(12aR)-10-Chloro-9-(2-fluoro-6-hydroxyphenyl)-7-(1H-imidazol-1-yl)-3,4,12,12a-tetrahydro-6H-pyrazino[2,1-c][1,4]benzoxazepin-2(1H)-yl]prop-2-en-1-one;
(12aR)-10-Chloro-9-(2-fluoro-6-hydroxyphenyl)-2-(prop-2-enoyl)-1,2,3,4,12,12a-hexahydro-6H-pyrazino[2,1-c][1,4]benzoxazepine-7-carbonitrile;
1-[(12aR)-10-Chloro-9-(2-fluoro-6-hydroxyphenyl)-7-(1H-pyrazol-1-yl)-3,4,12,12a-tetrahydro-6H-pyrazino[2,1-c][1,4]benzoxazepin-2(1H)-yl]prop-2-en-1-one;
1-((12aR)-10-Chloro-8-fluoro-9-(5-methyl-1H-benzo[d]imidazol-4-yl)-3,4,12,12a-tetrahydro-6H-benzo[f]pyrazino[2,1-c][1,4]oxazepin-2(1H)-yl) prop-2-en-1-one;
(12aR)-8,10-Dichloro-9-(2-fluoro-6-hydroxyphenyl)-2-(prop-2-enoyl)-1,2,3,4,12,12a-hexahydro-6H-pyrazino[2,1-c][1,4]benzoxazepin-6-one;
(12aR)-10-Chloro-9-(2-fluoro-6-hydroxyphenyl)-2-(prop-2-enoyl)-1,2,3,4,12,12a-hexahydro-6H-pyrazino[2,1-c][1,4]benzoxazepine-8-carbonitrile;
(12aR)-10-Chloro-9-(2-fluoro-6-hydroxyphenyl)-8-methyl-2-(prop-2-enoyl)-1,2,3,4,12,12a-hexahydro-6H-pyrazino[2,1-c][1,4]benzoxazepin-6-one;
1-[(12aR)-8,10-Dichloro-9-(2-fluoro-6-hydroxyphenyl)-3,4,12,12a-tetrahydro-6H-pyrazino[2,1-c][1,4]benzoxazepin-2(1H)-yl]prop-2-en-1-one;
8-[(12aR)-10-Chloro-8-fluoro-2-(prop-2-enoyl)-1,2,3,4,12,12a-hexahydro-6H-pyrazino[2,1-c][1,4]benzoxazepin-9-yl]-7-methylisoquinolin-1(2H)-one;
1-[(12aR)-10-chloro-9-(2-fluoro-6-hydroxyphenyl)-8-methoxy-3,4,12,12a-tetrahydro-6H-pyrazino[2,1-c][1,4]benzoxazepin-2(1H)-yl]prop-2-en-1-one;
(12aS)-10-Chloro-9-(5-methyl-1H-indazol-4-yl)-2-(prop-2-enoyl)-1,3,4,11,12,12a-hexahydropyrazino[2,1-c][1,4]benzodiazepin-6(2H)-one;
1-[(12aR)-10-chloro-9-(2-chloro-6-hydroxyphenyl)-8-fluoro-3,4,12,12a-tetrahydro-6H-pyrazino[2,1-c][1,4]benzoxazepin-2(1H)-yl]prop-2-en-1-one;
(12aS)-10-Chloro-11-methyl-9-(5-methyl-1H-indazol-4-yl)-2-(prop-2-enoyl)-1,3,4,11,12,12a-hexahydropyrazino[2,1-c][1,4]benzodiazepin-6(2H)-one;
1-[(12aR)-10-Chloro-9-(2,3-difluoro-6-hydroxyphenyl)-8-fluoro-3,4,12,12a-tetrahydro-6H-pyrazino[2,1-c][1,4]benzoxazepin-2(1H)-yl]prop-2-en-1-one;
(12aR)-10-Chloro-9-(2-hydroxy-6-methylphenyl)-2-(prop-2-enoyl)-1,2,3,4,12,12a-hexahydro-6H-pyrazino[2,1-c][1,4]benzoxazepine-8-carbonitrile;
1-[(12aR)-9-(2-Chloro-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-8,10-Difluoro-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-8,10-Difluoro-9-[2-fluoro-6-(hydroxymethyl)phenyl]-3,4,12,12a-tetrahydro-6H-pyrazino[2,1-c][1,4]benzoxazepin-2(1H)-yl]prop-2-en-1-one;
1-[(12aR)-8,10-Difluoro-9-[2-hydroxy-6-(trifluoromethyl)phenyl]-3,4,12,12a-tetrahydro-6H-pyrazino[2,1-c][1,4]benzoxazepin-2(1H)-yl]prop-2-en-1-one;
1-[(12aR)-9-(2-Ethyl-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-9-[2-(Difluoromethyl)-6-hydroxyphenyl]-8,10-difluoro-3,4,12,12a-tetrahydro-6H-pyrazino[2,1-c][1,4]benzoxazepin-2(1H)-yl]prop-2-en-1-one;
(12aR)-9-(2-Chloro-6-hydroxyphenyl)-10-fluoro-2-(prop-2-enoyl)-1,2,3,4,12,12a-hexahydro-6H-pyrazino[2,1-c][1,4]benzoxazepine-8-carbonitrile;
(12aR)-10-Chloro-9-(2-chloro-6-hydroxyphenyl)-2-(prop-2-enoyl)-1,2,3,4,12,12a-hexahydro-6H-pyrazino[2,1-c][1,4]benzoxazepine-8-carbonitrile;
1-[(12aR)-9-(2-Bromo-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-8-Chloro-10-fluoro-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-8-Chloro-10-ethynyl-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-10-Ethynyl-8-fluoro-9-(2-hydroxy-6-methylphenyl)-3,4,12,12a-tetrahydro-6H-pyrazino[2,1-c][1,4]benzoxazepin-2(1H)-yl]prop-2-en-1-one;
(6aR)-4-Chloro-3-(2-fluoro-6-hydroxyphenyl)-2-methyl-8-(prop-2-enoyl)-2,6,6a,7,8,9,10,12-octahydro-1H-pyrazino[2,1-c]pyrido[3,4-f][1,4]oxazepin-1-one;
1-[(6aR)-1,4-Dichloro-3-(2-fluoro-6-hydroxyphenyl)-6a,7,9,10-tetrahydro-12H-pyrazino[2,1-c]pyrido[3,4-f][1,4]oxazepin-8(6H)-yl]prop-2-en-1-one;
(6aR)-4-Chloro-3-(2-fluoro-6-hydroxyphenyl)-8-(prop-2-enoyl)-2,6,6a,7,8,9,10,12-octahydro-1H-pyrazino[2,1-c]pyrido[3,4-f][1,4]oxazepin-1-one;
1-[(8aR)-6-Chloro-5-(2-fluoro-6-hydroxyphenyl)-8a,9,11,12-tetrahydro-14H-pyrazino[2,1-c][1,2,4]triazolo[4′,3′:1,2]pyrido[3,4-f][1,4]oxazepin-10(8H)-yl]prop-2-en-1-one;
1-[(7aR)-5-chloro-4-(2-fluoro-6-hydroxyphenyl)-1-methyl-1,7a,8,10,11,13-hexahydropyrazino[2′,1′:3,4][1,4]oxazepino[7,6-g]indazol-9(7H)-yl]prop-2-en-1-one;
1-[(7aR)-5-Chloro-4-(2-fluoro-6-hydroxyphenyl)-2-methyl-2,7a,8,10,11,13-hexahydropyrazino[2′,1′:3,4][1,4]oxazepino[7,6-g]indazol-9(7H)-yl]prop-2-en-1-one;
1-[(12aR)-9-(2-chloro-6-hydroxyphenyl)-8-ethynyl-10-fluoro-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-ethynyl-3,4,12,12a-tetrahydro-6H-pyrazino[2,1-c][1,4]benzoxazepin-2(1H)-yl]prop-2-en-1-one;
1-((12aR)-10-chloro-8-ethynyl-9-(2-fluoro-6-hydroxyphenyl)-3,4,12,12a-tetrahydro-6H-benzo[f]pyrazino[2,1-c][1,4]oxazepin-2(1H)-yl)prop-2-en-1-one;
1-[(7aR)-5-Chloro-4-(2-chloro-6-hydroxyphenyl)-1-methyl-1,7a,8,10,11,13-hexahydroimidazo[4,5-g]pyrazino[2,1-c][1,4]benzoxazepin-9(7H)-yl]prop-2-en-1-one;
1-[(12aR)-8-Chloro-9-(2-chloro-6-hydroxyphenyl)-10-fluoro-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-10-fluoro-8-(prop-1-yn-1-yl)-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-2-ethynyl-6a,7,9,10-tetrahydro-12H-pyrazino[2,1-c]pyrido[2,3-f][1,4]oxazepin-8(6H)-yl]prop-2-en-1-one;
1-[(12aR)-9-(2-Chloro-6-hydroxyphenyl)-8-ethynyl-10-methyl-3,4,12,12a-tetrahydro-6H-pyrazino[2,1-c][1,4]benzoxazepin-2(1H)-yl]prop-2-en-1-one;
1-[(12aR)-10-chloro-9-(2-chloro-6-hydroxyphenyl)-7,8-difluoro-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-(difluoromethoxy)-10-fluoro-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-ethynyl-7,10-difluoro-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-(difluoromethoxy)-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-(cyclopropyloxy)-10-fluoro-3,4,12,12a-tetrahydro-6H-pyrazino[2,1-c][1,4]benzoxazepin-2(1H)-yl]prop-2-en-1-one;
1-((12aR)-9-(2-Chloro-6-hydroxyphenyl)-8-(3-(dimethylamino)prop-1-yn-1-yl)-10-fluoro-3,4,12,12a-tetrahydro-6H-benzo[f]pyrazino[2,1-c][1,4]oxazepin-2(1H)-yl)prop-2-en-1-one;
1-[(12aR)-9-(2-chloro-6-hydroxyphenyl)-10-fluoro-8-[(pyridin-4-yl)methoxy]-3,4,12,12a-tetrahydro-6H-pyrazino[2,1-c][1,4]benzoxazepin-2(1H)-yl]prop-2-en-1-one;
1-[(12aR)-10-chloro-9-(2-chloro-6-hydroxyphenyl)-8-(2-methoxyethoxy)-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-10-fluoro-8-[2-(piperidin-1-yl)ethoxy]-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-(prop-1-yn-1-yl)-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-2-[(²H₃)methyloxy]-6a,7,9,10-tetrahydro-12H-pyrazino[2,1-c]pyrido[2,3-f][1,4]oxazepin-8(6H)-yl]prop-2-en-1-one;
1-[(12aR)-10-Chloro-9-(2-chloro-6-hydroxyphenyl)-8-(methoxymethyl)-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-7-[2-(dimethylamino)ethoxy]-10-fluoro-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-1-(prop-1-yn-1-yl)-6a,7,9,10-tetrahydro-12H-pyrazino[2,1-c]pyrido[3,4-f][1,4]oxazepin-8(6H)-yl]prop-2-en-1-one; and
1-((6aR)-4-Chloro-3-(2-chloro-6-hydroxyphenyl)-1-ethynyl-6a,7,9,10-tetrahydro-12H-pyrazino[2,1-c]pyrido[3,4-f][1,4]oxazepin-8(6H)-yl)prop-2-en-1-one;
   or a pharmaceutically acceptable salt thereof;
   [10] the pharmaceutical product according to [2], wherein the RASG12C inhibitor is 1-[(12aR)-9-(2-chloro-6-hydroxyphenyl)-8-ethynyl-10-fluoro-3,4,12,12a-tetrahydro-6H-pyrazino[2,1-c][1,4]benzoxazepin-2(1H)-yl]prop-2-en-1-one;
   [11] the pharmaceutical product according to [2], wherein the RASG12C inhibitor is 1-[(12aR)-9-(2-chloro-6-hydroxyphenyl)-10-fluoro-8-(prop-1-yn-1-yl)-3,4,12,12a-tetrahydro-6H-pyrazino[2,1-c][1,4]benzoxazepin-2(1H)-yl]prop-2-en-1-one.
   [12] the pharmaceutical product according to [1], wherein the RASG12C inhibitor is selected from LY3537982 (Loxo/Lilly), AZD4625 (AstraZeneca), sotorasib (AMG510), adagrasib (MRTX849), JDQ443 (Novartis), GDC-6036 (Genentech), BI 1,823,911 (Boehringer Ingelheim), D1553 (InventisBio) and 74699157 (Johnson and Johnson), or a pharmaceutically acceptable salt thereof.
   [13] the pharmaceutical product according to [1], wherein the RASG12C inhibitor is Compound A represented by the following formula:

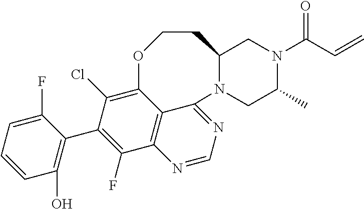 [SMILES: C=CC(=O)N1C[C@@H]2CCOc3c(Cl)c(-c4c(O)cccc4F)c(F)c4ncnc(c34)N2C[C@H]1C]

or a pharmaceutically acceptable salt thereof;
[14] the pharmaceutical product according to any one of [1] to [13], wherein the anti-HER2 antibody is an antibody comprising a heavy chain comprising CDRH1 consisting of an amino acid sequence represented by SEQ ID NO: 3 [=amino acid residues 26 to 33 of SEQ ID NO: 1], CDRH2 consisting of an amino acid sequence represented by SEQ ID NO: 4 [=amino acid residues 51 to 58 of SEQ ID NO: 1] and CDRH3 consisting of an amino acid sequence represented by SEQ ID NO: 5 [=amino acid residues 97 to 109 of SEQ ID NO: 1], and a light chain comprising CDRL1 consisting of an amino acid sequence represented by SEQ ID NO: 6 [=amino acid residues 27 to 32 of SEQ ID NO: 2], CDRL2 consisting of an amino acid sequence consisting of amino acid residues 1 to 3 of SEQ ID NO: 7 [=amino acid residues 50 to 52 of SEQ ID NO: 2] and CDRL3 consisting of an amino acid sequence represented by SEQ ID NO: 8 [=amino acid residues 89 to 97 of SEQ ID NO: 2];
[15] the pharmaceutical product according to any one of [1] to [13], wherein the anti-HER2 antibody is an antibody comprising a heavy chain comprising a heavy chain variable region consisting of an amino acid sequence represented by SEQ ID NO: 9 [=amino acid residues 1 to 120 of SEQ ID NO: 1] and a light chain comprising a light chain variable region consisting of an amino acid sequence represented by SEQ ID NO: 10 [=amino acid residues 1 to 107 of SEQ ID NO: 2];
[16] the pharmaceutical product according to any one of [1] to [13], wherein the anti-HER2 antibody is an antibody comprising a heavy chain consisting of an amino acid sequence represented by SEQ ID NO: 1 and a light chain consisting of an amino acid sequence represented by SEQ ID NO: 2;
[17] the pharmaceutical product according to any one of [1] to [13], wherein the anti-HER2 antibody is an antibody comprising a heavy chain consisting of an amino acid sequence represented by SEQ ID NO: 11 [=amino acid residues 1 to 449 of SEQ ID NO: 1] and a light chain consisting of an amino acid sequence represented by SEQ ID NO: 2;
[18] the pharmaceutical product according to any one of [1] to [17], wherein the antibody-drug conjugate is an antibody-drug conjugate in which a drug-linker represented by the following formula:

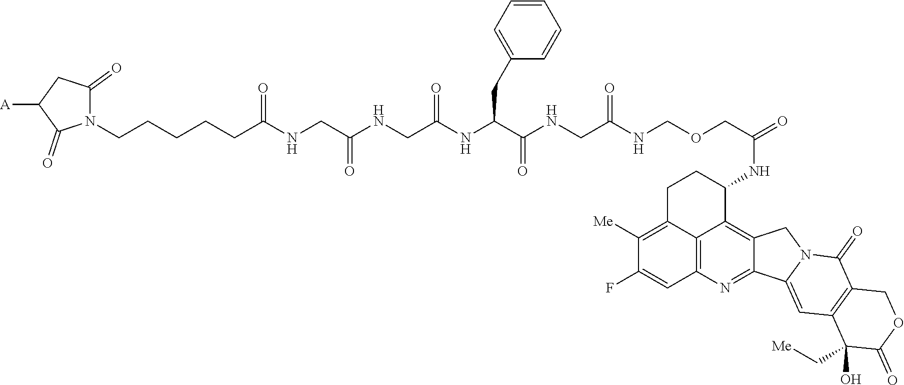

wherein A represents the connecting position to an antibody, is conjugated to an anti-HER2 antibody via a thioether bond; [19] the pharmaceutical product according to any one of [1] to [18], wherein the anti-HER2 antibody-drug conjugate is represented by the following formula:

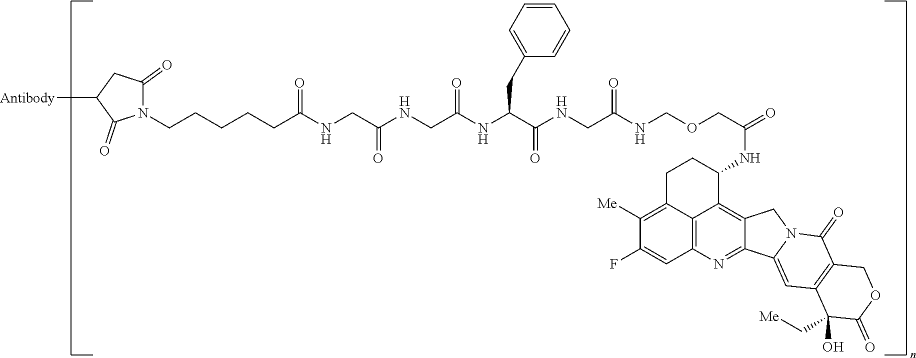

wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8;
[20] the pharmaceutical product according to any one of [1] to [13], wherein the anti-HER2 antibody-drug conjugate is trastuzumab deruxtecan (DS-8201) or trastuzumab emtansine (T-DM1);
[21] the pharmaceutical product according to [20], wherein the anti-HER2 antibody-drug conjugate is trastuzumab deruxtecan (DS-8201);
[22] the pharmaceutical product according to any one of [1] to [21] wherein the product is a composition comprising the anti-HER2 antibody-drug conjugate and the RASG12C inhibitor, for separate simultaneous administration;
[23] the pharmaceutical product according to any one of [1] to [21] wherein the product is a combined preparation comprising the anti-HER2 antibody-drug conjugate and the RASG12C inhibitor, for sequential administration;
[24] the pharmaceutical product according to any one of [1] to [23], wherein the product is for treating cancer;
[25] the pharmaceutical product according to [24], wherein the cancer is at least one selected from the group consisting of breast cancer, gastric cancer, colorectal cancer, lung cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, and melanoma;
[26] the pharmaceutical product according to [25], wherein the cancer is breast cancer;
[27] the pharmaceutical product according to [26], wherein the breast cancer has a HER2 status score of IHC 3+;
[28] the pharmaceutical product according to [26], wherein the breast cancer is HER2 low-expressing breast cancer;
[29] the pharmaceutical product according to [26], wherein the breast cancer has a HER2 status score of IHC 2+;
[30] the pharmaceutical product according to [26], wherein the breast cancer has a HER2 status score of IHC 1+;
[31] the pharmaceutical product according to [26], wherein the breast cancer has a HER2 status score of IHC>0 and <1+;
[32] the pharmaceutical product according to [26], wherein the breast cancer is triple-negative breast cancer;
[33] the pharmaceutical product according to [24], wherein the cancer is gastric cancer;
[34] the pharmaceutical product according to [24], wherein the cancer is colorectal cancer;
[35] the pharmaceutical product according to [24], wherein the cancer is lung cancer;
[36] the pharmaceutical product according to [35], wherein the lung cancer is non-small cell lung cancer;
[37] the pharmaceutical product according to [24], wherein the cancer is pancreatic cancer;
[38] the pharmaceutical product according to [24], wherein the cancer is ovarian cancer;
[39] the pharmaceutical product according to [24], wherein the cancer is prostate cancer;
[40] the pharmaceutical product according to [24], wherein the cancer is kidney cancer;
[41] a pharmaceutical product as defined in any one of [1] to [23], for use in treating cancer;
[42] the pharmaceutical product for the use according to [41], wherein the cancer is as defined in any one of [25] to [40];
[43] use of an anti-HER2 antibody-drug conjugate in the manufacture of a medicament for use in combination with a RASG12C inhibitor, wherein the anti-HER2 antibody-drug conjugate and the RASG12C inhibitor are as defined in any one of [1] to [21], for treating cancer;
[44] the use according to [43] wherein the medicament is for use in combination with the RASG12C inhibitor by sequential administration;
[45] the use according to [43] wherein the medicament is for use in combination with the RASG12C inhibitor by separate simultaneous administration;
[46] use of a RASG12C inhibitor in the manufacture of a medicament for use in combination with an anti-HER2 antibody-drug conjugate, wherein the anti-HER2 antibody-drug conjugate and the RASG12C inhibitor are as defined in any one of [1] to [21], for treating cancer;
[47] the use according to [46] wherein the medicament is for use in combination with the anti-HER2 antibody-drug conjugate by sequential administration;
[48] the use according to [46] wherein the medicament is for use in combination with the anti-HER2 antibody-drug conjugate by separate simultaneous administration.
[49] the use according to any one of [43] to [48], wherein the cancer is as defined in any one of [25] to [40];
[50] an anti-HER2 antibody-drug conjugate for use, in combination with a RASG12C inhibitor, in the treatment of cancer, wherein the anti-HER2 antibody-drug conjugate and the RASG12C inhibitor are as defined in any one of [1] to [21];
[51] the anti-HER2 antibody-drug conjugate for the use according to [50], wherein the cancer is as defined in any one of [25] to [40];
[52] the anti-HER2 antibody-drug conjugate for the use according to [50] or [51], wherein the use comprises administration of the anti-HER2 antibody-drug conjugate and the RASG12C inhibitor sequentially;
[53] the anti-HER2 antibody-drug conjugate for the use according to [50] or [51], wherein the use comprises administration of the anti-HER2 antibody-drug conjugate and the RASG12C inhibitor separately and simultaneously;
[54] An anti-HER2 antibody-drug conjugate for use in the treatment of cancer in a subject, wherein said treatment comprises the sequential or separate simultaneous administration of i) said anti-HER2 antibody-drug conjugate, and ii) a RASG12C inhibitor to said subject, wherein said anti-HER2 antibody-drug conjugate and said RASG12C inhibitor are as defined in any one of [1] to [21];
[55] a RASG12C inhibitor for use, in combination with an anti-HER2 antibody-drug conjugate, in the treatment of cancer, wherein the anti-HER2 antibody-drug conjugate and the RASG12C inhibitor are as defined in any one of [1] to [21];
[56] the RASG12C inhibitor for the use according to [55], wherein the cancer is as defined in any one of [25] to [40];
[57] the RASG12C inhibitor for the use according to [55] or [56], wherein the use comprises administration of the anti-HER2 antibody-drug conjugate and the RASG12C inhibitor sequentially;
[58] the RASG12C inhibitor for the use according to [55] or [56], wherein the use comprises administration of the anti-HER2 antibody-drug conjugate and the RASG12C inhibitor separately and simultaneously;
[59] A RASG12C inhibitor for use in the treatment of cancer in a subject, wherein said treatment comprises the sequential or separate simultaneous administration of i) said RASG12C inhibitor, and ii) an anti-HER2 antibody-drug conjugate to said subject, wherein said RASG12C inhibitor and said anti-HER2 antibody-drug conjugate are as defined in any one of [1] to [21];
[60] a method of treating cancer comprising administering an anti-HER2 antibody-drug conjugate and a RASG12C inhibitor as defined in any one of [1] to [21] in combination to a subject in need thereof;
[61] the method according to [60], wherein the cancer is as defined in any one of [25] to [40];
[62] the method according to [60] or [61], wherein the method comprises administering the anti-HER2 antibody-drug conjugate and the RASG12C inhibitor sequentially; and
[63] the method according to [60] or [61], wherein the method comprises administering the anti-HER2 antibody-drug conjugate and the RASG12C inhibitor separately and simultaneously.

### Advantageous Effects of Disclosure

The present disclosure provides a pharmaceutical product wherein an anti-HER2 antibody-drug conjugate, having an antitumor drug conjugated to an anti-HER2 antibody via a linker structure, and a RASG12C inhibitor are administered in combination, and a therapeutic use and method wherein the specific antibody-drug conjugate and the RASG12C inhibitor are administered in combination to a subject. Thus, the present disclosure can provide a medicine and treatment which can obtain a superior antitumor effect in the treatment of cancers.

### BRIEF DESCRIPTION OF DRAWINGS

FIG. 1 is a diagram showing the amino acid sequence of a heavy chain of an anti-HER2 antibody (SEQ ID NO: 1).

FIG. 2 is a diagram showing the amino acid sequence of a light chain of an anti-HER2 antibody (SEQ ID NO: 2).

FIG. 3 is a diagram showing the amino acid sequence of a heavy chain CDRH1 (SEQ ID NO: 3 [=amino acid residues 26 to 33 of SEQ ID NO: 1]).

FIG. 4 is a diagram showing the amino acid sequence of a heavy chain CDRH2 (SEQ ID NO: 4 [=amino acid residues 51 to 58 of SEQ ID NO: 1]).

FIG. 5 is a diagram showing the amino acid sequence of a heavy chain CDRH3 (SEQ ID NO: 5 [=amino acid residues 97 to 109 of SEQ ID NO: 1]).

FIG. 6 is a diagram showing the amino acid sequence of a light chain CDRL1 (SEQ ID NO: 6 [=amino acid residues 27 to 32 of SEQ ID NO: 2]).

FIG. 7 is a diagram showing an amino acid sequence comprising the amino acid sequence of a light chain CDRL2 (SAS) (SEQ ID NO: 7 [=amino acid residues 50 to 56 of SEQ ID NO: 2]).

FIG. 8 is a diagram showing the amino acid sequence of a light chain CDRL3 (SEQ ID NO: 8 [=amino acid residues 89 to 97 of SEQ ID NO: 2]).

FIG. 9 is a diagram showing the amino acid sequence of a heavy chain variable region (SEQ ID NO: 9 [=amino acid residues 1 to 120 of SEQ ID NO: 1]).

FIG. 10 is a diagram showing the amino acid sequence of a light chain variable region (SEQ ID NO: 10 [=amino acid residues 1 to 107 of SEQ ID NO: 2]).

FIG. 11 is a diagram showing the amino acid sequence of a heavy chain (SEQ ID NO: 11 [=amino acid residues 1 to 449 of SEQ ID NO: 1]).

FIG. 12 shows combination matrices for combining Compound A with DS-8201 in KRAS^{G12C} lung cancer cell lines H358 and H2122, and in KRAS^{G12C} oesophageal cell line KYSE410.

FIG. 13 shows graphs representing effects of combinations of Compound A with DS-8201 on tumour growth inhibition and regrowth on treatment cessation in vitro in eight KRAS^{G12C} cancer cell lines.

FIG. 14 shows a graph representing effects of combinations of Compound A with DS-8201 on tumour growth inhibition in vivo in KRAS^{G12C} mutant lung cancer xenograft model NCI-H358.

FIG. 15 shows a graph representing effects of combinations of Compound A with DS-8201 on tumour growth inhibition in vivo in KRAS^{G12C} mutant lung cancer xenograft model NCI-H2122.

FIG. 16 shows a graph representing effects of combinations of Compound A with DS-8201 on tumour growth inhibition in vivo in KRAS^{G12C} mutant lung cancer xenograft model LU99.

FIG. 17 shows a graph representing effects of combinations of Compound A with DS-8201 on tumour growth inhibition in vivo in KRAS^{G12C} mutant colorectal cancer patient derived explant model CTG-1489.

FIG. 18 a graph representing effects of combinations of Compound A with DS-8201 on tumour growth inhibition in vivo in KRAS^{G12C} mutant colorectal cancer patient derived explant model CTG-0387.

FIG. 19A shows combination matrices for combining Compound A, AMG510 or MRTX849 with T-DM1 in KRAS^{G12C} lung cancer cell line H358. FIG. 19B shows combination matrices for combining Compound A, AMG510 or MRTX849 with trastuzumab in KRAS^{G12C} lung cancer cell line H358.

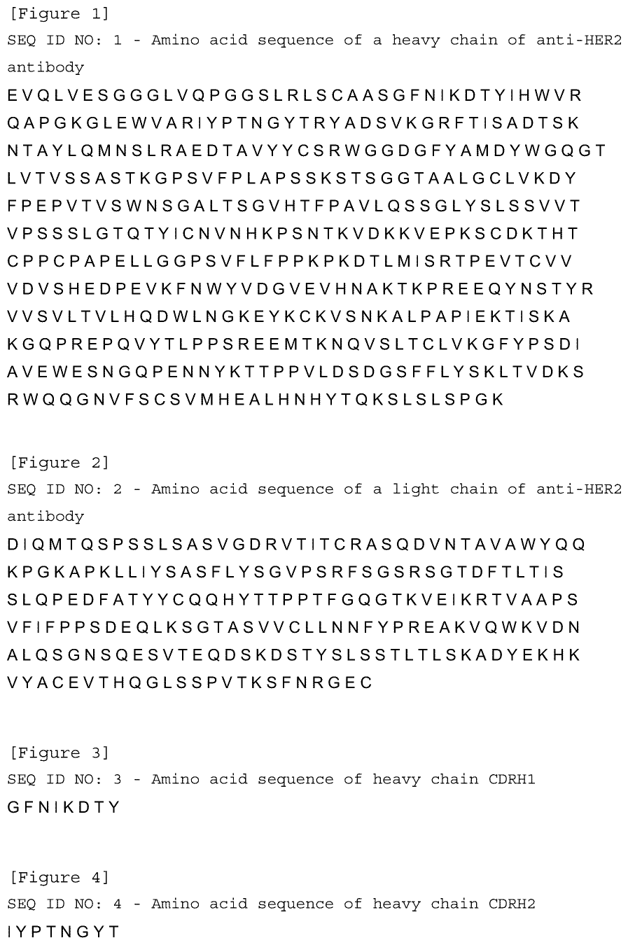

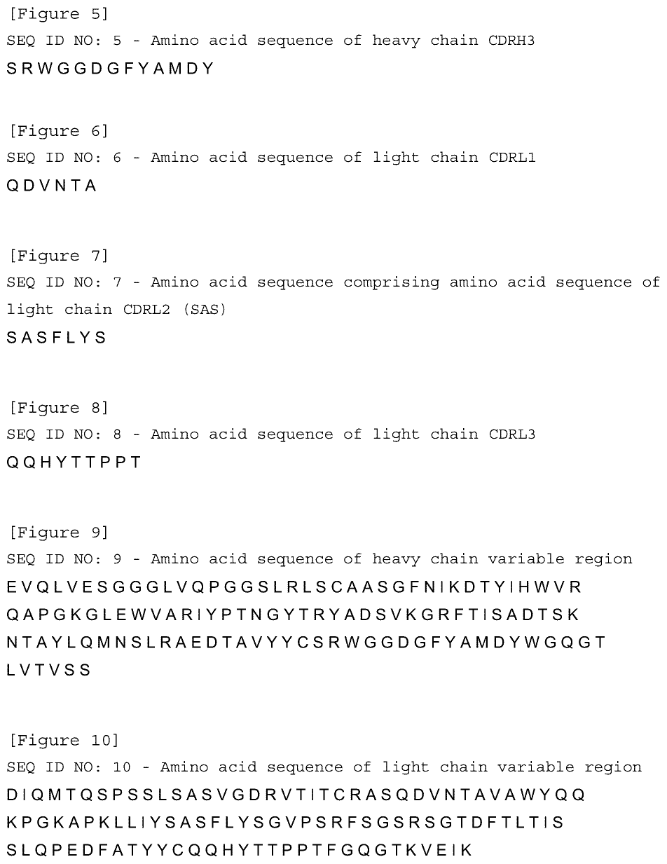

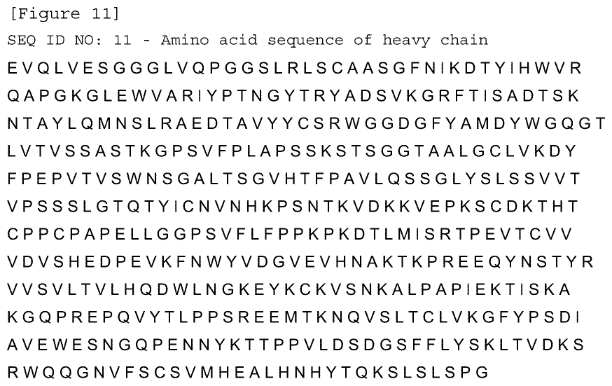

In order that the present disclosure can be more readily understood, certain terms are first defined. Additional definitions are set forth throughout the detailed description.

Before describing the present disclosure in detail, it is to be understood that this disclosure is not limited to specific compositions or method steps, as such can vary. As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range.

It is understood that wherever aspects are described herein with the language “comprising”, otherwise analogous aspects described in terms of “consisting of” and/or “consisting essentially of” are also provided.

The terms “inhibit”, “block”, and “suppress” are used interchangeably herein and refer to any statistically significant decrease in biological activity, including full blocking of the activity. For example, “inhibition” can refer to a decrease of about 10%, 20%, 30%, 40%, 50%, 60%, 70%, 80%, 90% or 100% in biological activity. Cellular proliferation can be assayed using art recognized techniques which measure rate of cell division, and/or the fraction of cells within a cell population undergoing cell division, and/or rate of cell loss from a cell population due to terminal differentiation or cell death (e.g., thymidine incorporation).

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical product” refers to a preparation which is in such form as to permit the biological activity of the active ingredients, either as a composition containing all the active ingredients (for simultaneous administration), or as a combination of separate compositions (a combined preparation) each containing at least one but not all of the active ingredients (for administration sequentially or simultaneously), and which contains no additional components which are unacceptably toxic to a subject to which the product would be administered. Such product can be sterile. By “simultaneous administration” is meant that the active ingredients are administered at the same time. By “sequential administration” is meant that the active ingredients are administered one after the other, in either order, at a time interval between the individual administrations. The time interval can be, for example, less than 24 hours, preferably less than 6 hours, more preferably less than 2 hours.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain aspects, a subject is successfully “treated” for cancer according to the methods of the present disclosure if the patient shows, e.g., total, partial, or transient remission of a certain type of cancer.

The terms “cancer”, “tumor”, “cancerous”, and “malignant” refer to or describe the physiological condition in mammals that is typically characterized by unregulated cell growth. Examples of cancers include but are not limited to, breast cancer, gastric cancer, colorectal cancer, lung cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, and melanoma. Cancers include hematological malignancies such as acute myeloid leukemia, multiple myeloma, chronic lymphocytic leukemia, diffuse large B cell lymphoma, Burkitt's lymphoma, follicular lymphoma and solid tumors such as breast cancer, lung cancer, neuroblastoma and colon cancer.

The term “cytotoxic agent” as used herein is defined broadly and refers to a substance that inhibits or prevents the function of cells and/or causes destruction of cells (cell death), and/or exerts anti-neoplastic/anti-proliferative effects. For example, a cytotoxic agent prevents directly or indirectly the development, maturation, or spread of neoplastic tumor cells. The term includes also such agents that cause a cytostatic effect only and not a mere cytotoxic effect. The term includes chemotherapeutic agents as specified below, as well as other HER2 antagonists, anti-angiogenic agents, tyrosine kinase inhibitors, protein kinase A inhibitors, members of the cytokine family, radioactive isotopes, and toxins such as enzymatically active toxins of bacterial, fungal, plant or animal origin.

The term “chemotherapeutic agent” is a subset of the term “cytotoxic agent” comprising natural or synthetic chemical compounds.

In accordance with the methods or uses of the present disclosure, compounds of the present disclosure may be administered to a patient to promote a positive therapeutic response with respect to cancer. The term “positive therapeutic response” with respect to cancer treatment refers to an improvement in the symptoms associated with the disease. For example, an improvement in the disease can be characterized as a complete response. The term “complete response” refers to an absence of clinically detectable disease with normalization of any previous test results. Alternatively, an improvement in the disease can be categorized as being a partial response. A “positive therapeutic response” encompasses a reduction or inhibition of the progression and/or duration of cancer, the reduction or amelioration of the severity of cancer, and/or the amelioration of one or more symptoms thereof resulting from the administration of compounds of the present disclosure. In specific aspects, such terms refer to one, two or three or more results following the administration of compounds of the instant disclosure:
(1) a stabilization, reduction or elimination of the cancer cell population;
(2) a stabilization or reduction in cancer growth;
(3) an impairment in the formation of cancer;
(4) eradication, removal, or control of primary, regional and/or metastatic cancer;
(5) a reduction in mortality;
(6) an increase in disease-free, relapse-free, progression-free, and/or overall survival, duration, or rate;
(7) an increase in the response rate, the durability of response, or number of patients who respond or are in remission;
(8) a decrease in hospitalization rate,
(9) a decrease in hospitalization lengths,
(10) the size of the cancer is maintained and does not increase or increases by less than 10%, preferably less than 5%, preferably less than 4%, preferably less than 2%, and
(11) an increase in the number of patients in remission.
(12) a decrease in the number of adjuvant therapies (e.g., chemotherapy or hormonal therapy) that would otherwise be required to treat the cancer.

Clinical response can be assessed using screening techniques such as PET, magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like. In addition to these positive therapeutic responses, the subject undergoing therapy can experience the beneficial effect of an improvement in the symptoms associated with the disease.

Alkyl groups and moieties are straight or branched chain, e.g. C₁₋₈ alkyl, C₁₋₆ alkyl, C₁₋₄ alkyl or C₅₋₆ alkyl. Examples of alkyl groups are methyl, ethyl, n-propyl, iso-propyl, n-butyl, t-butyl, n-pentyl, n-hexyl, n-heptyl and n-octyl, such as methyl or n-hexyl.

Fluoroalkyl groups are alkyl groups in which one or more H atoms is replaced with one or more fluoro atoms, e.g. C₁₋₈ fluoroalkyl, C₁₋₆ fluoroalkyl, C₁₋₄ fluoroalkyl or C₅₋₆ fluoroalkyl. Examples include fluoromethyl (CH₂F—), difluromethyl (CHF₂—), trifluoromethyl (CF₃—), 2,2,2-trifluoroethyl (CF₃CH₂—), 1,1-difluoroethyl (CH₃CHF₂—), 2,2-difluoroethyl (CHF₂CH₂—), and 2-fluoroethyl (CH₂FCH₂—).

Halo means fluoro, chloro, bromo, and iodo. In an embodiment, halo is fluoro or chloro.

As used herein, the phrase “effective amount” means an amount of a compound or composition which is sufficient enough to significantly and positively modify the symptoms and/or conditions to be treated (e.g., provide a positive clinical response). The effective amount of an active ingredient for use in a pharmaceutical product will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician. In particular, an effective amount of a compound for use in the treatment of cancer in combination with the antibody-drug conjugate is an amount such that the combination is sufficient to symptomatically relieve in a warm-blooded animal such as man, the symptoms of cancer, to slow the progression of cancer, or to reduce in patients with symptoms of cancer the risk of getting worse.

In this specification, unless otherwise stated, the term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

It will be understood that compounds of formula (I) or (II) may form stable pharmaceutically acceptable acid or base salts, and in such cases administration of a compound as a salt may be appropriate. Examples of acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Non-toxic physiologically-acceptable salts are preferred, although other salts may be useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

Compounds of formula (I) or (II) may have more than one chiral center, and it is to be understood that the application encompasses all individual stereoisomers, enantiomers and diastereoisomers and mixtures thereof. Thus, it is to be understood that, insofar as the compounds of formula (I) or (II) can exist in optically active or racemic forms by virtue of one or more asymmetric carbon atoms, the application includes in its definition any such optically active or racemic form which possesses the above-mentioned activity. The present application encompasses all such stereoisomers having activity as herein defined.

Thus, throughout the specification, where reference is made to the compound of formula (I) or (II) it is to be understood that the term compound includes diastereoisomers, mixtures of diastereoisomers, and enantiomers that are RASG12C inhibitors.

It is also to be understood that certain compounds of formula (I) or (II), and pharmaceutically salts thereof, can exist in solvated as well as unsolvated forms such as, for example, hydrated and anhydrous forms. It is to be understood that the compounds herein encompass all such solvated forms. For the sake of clarity, this includes both solvated (e.g., hydrated) forms of the free form of the compound, as well as solvated (e.g., hydrated) forms of the salt of the compound.

Some of the compounds of formula (I) or (II) may be crystalline and may have more than one crystalline form. It is to be understood that the disclosure encompasses any crystalline or amorphous form, or mixtures thereof, which have RASG12C inhibitory activity. It is generally known that crystalline materials may be analysed using conventional techniques such as, for example, X-Ray Powder Diffraction (hereinafter XRPD) analysis and Differential Scanning Calorimetry (DSC).

Formula (I) or (II) as described herein is intended to encompass all isotopes of its constituent atoms. For example, H (or hydrogen) includes any isotopic form of hydrogen including ¹H, ²H (D), and ³H (T); C includes any isotopic form of carbon including ¹²C, ¹³C, and ¹⁴C; O includes any isotopic form of oxygen including ¹⁶O, ¹⁷O and ¹⁸O; N includes any isotopic form of nitrogen including ¹³N, ¹⁴N and ¹⁵N; F includes any isotopic form of fluorine including ¹⁹F and ¹⁸F; and the like. In one aspect, the compounds of formula (I) or (II) include isotopes of the atoms covered therein in amounts corresponding to their naturally occurring abundance. However, in certain instances, it may be desirable to enrich one or more atom in a particular isotope which would normally be present in a lower abundance. For example, ¹H would normally be present in greater than 99.98% abundance; however, in one aspect, a compound of any formula presented herein may be enriched in ²H or ³H at one or more positions where H is present. In another aspect, when a compound of any formula presented herein is enriched in a radioactive isotope, for example ³H and ¹⁴C, the compound may be useful in drug and/or substrate tissue distribution assays. It is to be understood that the present application encompasses all such isotopic forms.

### DESCRIPTION OF EMBODIMENTS

Hereinafter, preferred modes for carrying out the present disclosure are described. The embodiments described below are given merely for illustrating one example of a typical embodiment of the present disclosure and are not intended to limit the scope of the present disclosure.

### 1. Antibody-Drug Conjugate

The antibody-drug conjugate used in the present disclosure is an antibody-drug conjugate in which the antibody is an anti-HER2 antibody, conjugated to a drug by a linker. The anti-HER2 antibody-drug conjugate used in the present disclosure is not particularly limited, and may be selected from a list including, e.g., trastuzumab deruxtecan, trastuzumab emtansine, trastuzumab duocarmazine (SYD985), A166, XMT-1522, RC48, ALT-P7, ARX788, PF-06804103, MRG002, ZW49, and BDC-1001. Preferably, the antibody-drug conjugate used in the present disclosure is an antibody-drug conjugate such as trastuzumab deruxtecan, in which a drug-linker represented by the following formula:

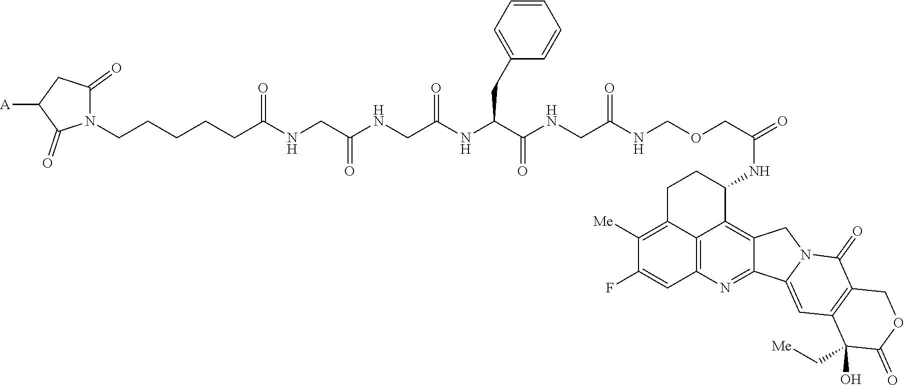

wherein A represents the connecting position to an antibody, is conjugated to an anti-HER2 antibody via a thioether bond. Alternatively, the antibody-drug conjugate may be trastuzumab emtansine (T-DM1).

In the present disclosure, the partial structure consisting of a linker and a drug in the antibody-drug conjugate is referred to as a “drug-linker”. The drug-linker may be connected to a thiol group (in other words, the sulfur atom of a cysteine residue) formed at an interchain disulfide bond site (two sites between heavy chains, and two sites between a heavy chain and a light chain) in the antibody.

For example, the drug-linker may include exatecan (IUPAC name: (1S,9S)-1-amino-9-ethyl-5-fluoro-1,2,3,9,12,15-hexahydro-9-hydroxy-4-methyl-10H,13H-benzo[de]pyrano[3′,4′:6,7]indolizino[1,2-b]quinolin-10,13-dione, (also expressed as chemical name: (1S,9S)-1-amino-9-ethyl-5-fluoro-2,3-dihydro-9-hydroxy-4-methyl-1H,12H-benzo[de]pyrano[3′,4′:6,7]indolizino[1,2-b]quinolin-10,13(9H,15H)-dione)), which is a topoisomerase I inhibitor, as a component. Exatecan is a camptothecin derivative having an antitumor effect, represented by the following formula:

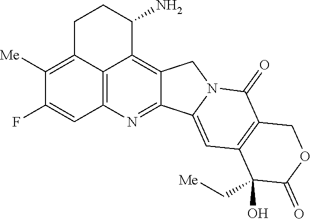 [SMILES: CC[C@@]1(O)C(=O)OCc2c1cc1n(c2=O)Cc2c-1nc1cc(F)c(C)c3c1c2[C@@H](N)CC3]

A preferred anti-HER2 antibody-drug conjugate used in the present disclosure can be also represented by the following formula:

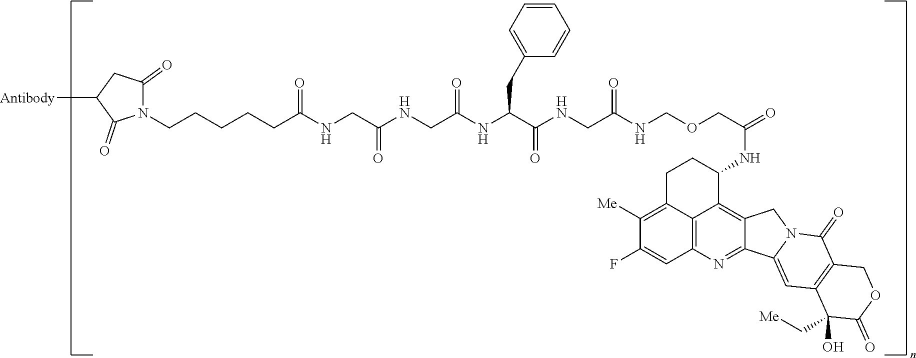

Here, the drug-linker is conjugated to an anti-HER2 antibody (‘Antibody-’) via a thioether bond. The meaning of n is the same as that of what is called the average number of conjugated drug molecules (DAR; Drug-to-Antibody Ratio), and indicates the average number of units of the drug-linker conjugated per antibody molecule.

After migrating into cancer cells, a preferred anti-HER2 antibody-drug conjugate such as trastuzumab deruxtecan may be cleaved at the linker portion to release a compound represented by the following formula:

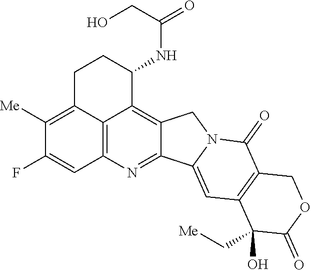 [SMILES: CC[C@@]1(O)C(=O)OCc2c1cc1n(c2=O)Cc2c-1nc1cc(F)c(C)c3c1c2[C@@H](NC(=O)CO)CC3]

This compound is inferred to be the original source of the antitumor activity of a preferred anti-HER2 antibody-drug conjugate such as trastuzumab deruxtecan, and has been confirmed to have a topoisomerase I inhibitory effect (Ogitani Y. et al., Clinical Cancer Research, 2016, Oct. 15; 22(20):5097-5108, Epub 2016 Mar. 29).

A preferred anti-HER2 antibody-drug conjugate trastuzumab deruxtecan is known to have a bystander effect (Ogitani Y. et al., Cancer Science (2016) 107, 1039-1046). The bystander effect is exerted through a process whereby the antibody-drug conjugate used in the present disclosure is internalized in cancer cells expressing the target and the compound released then exerts an antitumor effect also on cancer cells which are present therearound and not expressing the target. This bystander effect may be exerted as an excellent antitumor effect even when the anti-HER2 antibody-drug conjugate is used in combination with a RASG12C inhibitor according to the present disclosure.

In alternative embodiments, the antibody-drug conjugate may include a maytansinoid, such as mertansine (DM1), or another microtubule inhibitor as a component. In certain embodiments, the maytansinoid may be linked to the antibody through a non-cleavable thioether linker, such as succinimidyl 4-(N-maleimidomethyl) cyclohexane-1-carboxylate (SMCC). In other embodiments, the antibody-drug conjugate may include a DNA alkylating agent (such as duocarmycin), an auristatin derivative (such as duostatin-5, monomethyl auristatin E (MMAE), or AUR-06380101), a tubulin inhibitor (such as AS269), or a TLR 7/8 agonist as a component.

### 2. Antibody in Antibody-Drug Conjugate

The anti-HER2 antibody in the antibody-drug conjugate used in the present disclosure may be derived from any species, and is preferably an anti-HER2 antibody derived from a human, a rat, a mouse, or a rabbit. In cases when the antibody is derived from species other than human species, it is preferably chimerized or humanized using a well known technique. The anti-HER2 antibody may be a polyclonal antibody or a monoclonal antibody and is preferably a monoclonal antibody.

The antibody in the antibody-drug conjugate used in the present disclosure is an anti-HER2 antibody preferably having a characteristic of being capable of targeting cancer cells, and is preferably an antibody possessing, for example, a property of recognizing a cancer cell, a property of binding to a cancer cell, a property of internalizing in a cancer cell, and/or cytocidal activity against cancer cells.

The binding activity of the anti-HER2 antibody against cancer cells can be confirmed using flow cytometry. The internalization of the antibody into cancer cells can be confirmed using (1) an assay of visualizing an antibody incorporated in cells under a fluorescence microscope using a secondary antibody (fluorescently labeled) binding to the therapeutic antibody (Cell Death and Differentiation (2008) 15, 751-761), (2) an assay of measuring a fluorescence intensity incorporated in cells using a secondary antibody (fluorescently labeled) binding to the therapeutic antibody (Molecular Biology of the Cell, Vol. 15, 5268-5282, December 2004), or (3) a Mab-ZAP assay using an immunotoxin binding to the therapeutic antibody wherein the toxin is released upon incorporation into cells to inhibit cell growth (Bio Techniques 28: 162-165, January 2000). As the immunotoxin, a recombinant complex protein of a diphtheria toxin catalytic domain and protein G may be used.

The antitumor activity of the anti-HER2 antibody can be confirmed in vitro by determining inhibitory activity against cell growth. For example, a cancer cell line overexpressing HER2 as a target protein for the antibody is cultured, and the antibody is added at varying concentrations into the culture system to determine inhibitory activity against focus formation, colony formation, and spheroid growth. The antitumor activity can be confirmed in vivo, for example, by administering the antibody to a nude mouse with a transplanted cancer cell line highly expressing the target protein, and determining change in the cancer cell.

Since the compound conjugated in the anti-HER2 antibody-drug conjugate exerts an antitumor effect, it is preferred but not essential that the anti-HER2 antibody itself should have an antitumor effect. For the purpose of specifically and selectively exerting the cytotoxic activity of the antitumor compound against cancer cells, it is important and also preferred that the anti-HER2 antibody should have the property of internalizing to migrate into cancer cells.

The anti-HER2 antibody in the antibody-drug conjugate used in the present disclosure can be obtained by a procedure known in the art. For example, the antibody of the present disclosure can be obtained using a method usually carried out in the art, which involves immunizing animals with an antigenic polypeptide and collecting and purifying antibodies produced in vivo. The origin of the antigen is not limited to humans, and the animals may be immunized with an antigen derived from a non-human animal such as a mouse, a rat and the like. In this case, the cross-reactivity of antibodies binding to the obtained heterologous antigen with human antigens can be tested to screen for an antibody applicable to a human disease.

Alternatively, antibody-producing cells which produce antibodies against the antigen are fused with myeloma cells according to a method known in the art (e.g., Kohler and Milstein, Nature (1975) 256, p. 495-497; and Kennet, R. ed., Monoclonal Antibodies, p. 365-367, Plenum Press, N.Y. (1980)) to establish hybridomas, from which monoclonal antibodies can in turn be obtained.

The antigen can be obtained by genetically engineering host cells to produce a gene encoding the antigenic protein. Specifically, vectors that permit expression of the antigen gene are prepared and transferred to host cells so that the gene is expressed. The antigen thus expressed can be purified. The antibody can also be obtained by a method of immunizing animals with the above-described genetically engineered antigen-expressing cells or a cell line expressing the antigen.

The anti-HER2 antibody in the antibody-drug conjugate used the present disclosure is preferably a recombinant antibody obtained by artificial modification for the purpose of decreasing heterologous antigenicity to humans such as a chimeric antibody or a humanized antibody, or is preferably an antibody having only the gene sequence of an antibody derived from a human, that is, a human antibody. These antibodies can be produced using a known method.

As the chimeric antibody, an antibody in which antibody variable and constant regions are derived from different species, for example, a chimeric antibody in which a mouse- or rat-derived antibody variable region is connected to a human-derived antibody constant region can be exemplified (Proc. Natl. Acad. Sci. USA, 81, 6851-6855, (1984)).

As the humanized antibody, an antibody obtained by integrating only the complementarity determining region (CDR) of a heterologous antibody into a human-derived antibody (Nature (1986) 321, pp. 522-525), and an antibody obtained by grafting a part of the amino acid residues of the framework of a heterologous antibody as well as the CDR sequence of the heterologous antibody to a human antibody by a CDR-grafting method (WO 90/07861), and an antibody humanized using a gene conversion mutagenesis strategy (U.S. Pat. No. 5,821,337) can be exemplified.

As the human antibody, an antibody generated by using a human antibody-producing mouse having a human chromosome fragment including genes of a heavy chain and light chain of a human antibody (see Tomizuka, K. et al., Nature Genetics (1997) 16, p. 133-143; Kuroiwa, Y. et. al., Nucl. Acids Res. (1998) 26, p. 3447-3448; Yoshida, H. et. al., Animal Cell Technology: Basic and Applied Aspects vol. 10, p. 69-73 (Kitagawa, Y., Matsuda, T. and Iijima, S. eds.), Kluwer Academic Publishers, 1999; Tomizuka, K. et. al., Proc. Natl. Acad. Sci. USA (2000) 97, p. 722-727, etc.) can be exemplified. As an alternative, an antibody obtained by phage display, the antibody being selected from a human antibody library (see Wormstone, I. M. et. al, Investigative Ophthalmology & Visual Science. (2002)43 (7), p. 2301-2308; Carmen, S. et. al., Briefings in Functional Genomics and Proteomics (2002), 1(2), p. 189-203; Siriwardena, D. et. al., Ophthalmology (2002) 109(3), p. 427-431, etc.) can be exemplified.

In the present disclosure, modified variants of the anti-HER2 antibody in the antibody-drug conjugate used in the present disclosure are also included. The modified variant refers to a variant obtained by subjecting the antibody according to the present disclosure to chemical or biological modification. Examples of the chemically modified variant include variants including a linkage of a chemical moiety to an amino acid skeleton, variants including a linkage of a chemical moiety to an N-linked or O-linked carbohydrate chain, etc. Examples of the biologically modified variant include variants obtained by post-translational modification (such as N-linked or O-linked glycosylation, N- or C-terminal processing, deamidation, isomerization of aspartic acid, or oxidation of methionine), and variants in which a methionine residue has been added to the N terminus by being expressed in a prokaryotic host cell. Further, an antibody labeled so as to enable the detection or isolation of the antibody or an antigen according to the present disclosure, for example, an enzyme-labeled antibody, a fluorescence-labeled antibody, and an affinity-labeled antibody are also included in the meaning of the modified variant. Such a modified variant of the antibody according to the present disclosure is useful for improving the stability and blood retention of the antibody, reducing the antigenicity thereof, detecting or isolating an antibody or an antigen, and so on.

Further, by regulating the modification of a glycan which is linked to the antibody according to the present disclosure (glycosylation, defucosylation, etc.), it is possible to enhance antibody-dependent cellular cytotoxic activity. As the technique for regulating the modification of a glycan of antibodies, those disclosed in WO99/54342, WO00/61739, WO02/31140, WO2007/133855, WO2013/120066, etc. are known. However, the technique is not limited thereto. In the anti-HER2 antibody according to the present disclosure, antibodies in which the modification of a glycan is regulated are also included.

It is known that a lysine residue at the carboxyl terminus of the heavy chain of an antibody produced in a cultured mammalian cell is deleted (Journal of Chromatography A, 705: 129-134 (1995)), and it is also known that two amino acid residues (glycine and lysine) at the carboxyl terminus of the heavy chain of an antibody produced in a cultured mammalian cell are deleted and a proline residue newly located at the carboxyl terminus is amidated (Analytical Biochemistry, 360: 75-83 (2007)). However, such deletion and modification of the heavy chain sequence do not affect the antigen-binding affinity and the effector function (the activation of complement, antibody-dependent cellular cytotoxicity, etc.) of the antibody. Therefore, in the anti-HER2 antibody according to the present disclosure, antibodies subjected to such modification and functional fragments of the antibody are also included, and deletion variants in which one or two amino acids have been deleted at the carboxyl terminus of the heavy chain, variants obtained by amidation of deletion variants (for example, a heavy chain in which the carboxyl terminal proline residue has been amidated), and the like are also included. The type of deletion variant having a deletion at the carboxyl terminus of the heavy chain of the anti-HER2 antibody according to the present disclosure is not limited to the above variants as long as the antigen-binding affinity and the effector function are conserved. The two heavy chains constituting the antibody according to the present disclosure may be of one type selected from the group consisting of a full-length heavy chain and the above-described deletion variant, or may be of two types in combination selected therefrom. The ratio of the amount of each deletion variant can be affected by the type of cultured mammalian cells which produce the anti-HER2 antibody according to the present disclosure and the culture conditions; however, an antibody in which one amino acid residue at the carboxyl terminus has been deleted in both of the two heavy chains in the antibody according to the present disclosure can be exemplified as preferred.

As isotypes of the anti-HER2 antibody according to the present disclosure, for example, IgG (IgG1, IgG2, IgG3, IgG4) can be exemplified, and IgG1 or IgG2 can be exemplified as preferred.

In the present disclosure, the term “anti-HER2 antibody” refers to an antibody which specifically binds to HER2 (Human Epidermal Growth Factor Receptor Type 2; ErbB-2), and preferably has an activity of internalizing in HER2-expressing cells by binding to HER2.

Examples of the anti-HER2 antibody include trastuzumab (U.S. Pat. No. 5,821,337) and pertuzumab (WO01/00245), and trastuzumab can be exemplified as preferred. In alternative embodiments, the anti-HER2 antibody may be selected from a list including hertuzumab, HT-19, MAB802, and ZW25.

### 3. Production of Antibody-Drug Conjugate

A preferred drug-linker intermediate for use in production of the anti-HER2 antibody-drug conjugate according to the present disclosure is represented by the following formula:

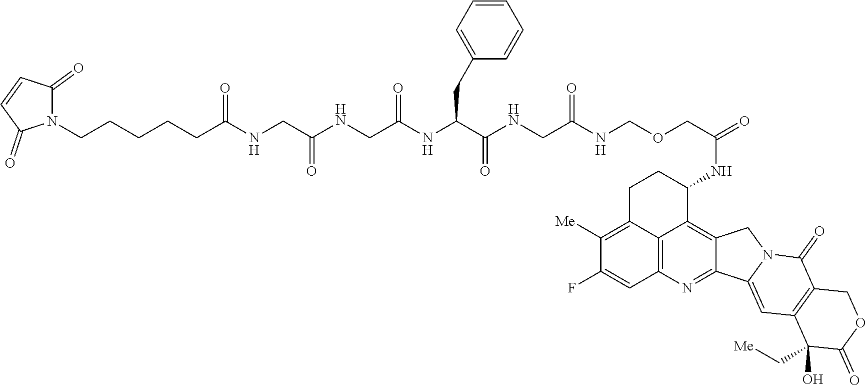 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

This drug-linker intermediate can be expressed as the chemical name N-[6-(2,5-dioxo-2,5-dihydro-1H-pyrrol-1-yl)hexanoyl]glycylglycyl-L-phenylalanyl-N-[(2-{[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]amino}-2-oxoethoxy)methyl]glycinamide, and can be produced with reference to descriptions in WO2014/057687, WO2015/098099, WO2015/115091, WO2015/155998, WO2019/044947 and so on.

A preferred anti-HER2 antibody-drug conjugate such as trastuzumab deruxtecan can be produced by reacting the above-described drug-linker intermediate and an anti-HER2 antibody having a thiol group (also referred to as a sulfhydryl group).

The anti-HER2 antibody having a sulfhydryl group can be obtained by a method well known in the art (Hermanson, G. T, Bioconjugate Techniques, pp. 56-136, pp. 456-493, Academic Press (1996)). For example, by using 0.3 to 3 molar equivalents of a reducing agent such as tris(2-carboxyethyl)phosphine hydrochloride (TCEP) per interchain disulfide within the antibody and reacting with the antibody in a buffer solution containing a chelating agent such as ethylenediamine tetraacetic acid (EDTA), an anti-HER2 antibody having a sulfhydryl group with partially or completely reduced interchain disulfides within the antibody can be obtained.

Further, by using 2 to 20 molar equivalents of the drug-linker intermediate per anti-HER2 antibody having a sulfhydryl group, an anti-HER2 antibody-drug conjugate in which 2 to 8 drug molecules are conjugated per antibody molecule can be produced.

The average number of conjugated drug molecules per anti-HER2 antibody molecule of the antibody-drug conjugate produced can be determined, for example, by a method of calculation based on measurement of UV absorbance for the antibody-drug conjugate and the conjugation precursor thereof at two wavelengths of 280 nm and 370 nm (UV method), or a method of calculation based on quantification through HPLC measurement for fragments obtained by treating the antibody-drug conjugate with a reducing agent (HPLC method).

Conjugation between the anti-HER2 antibody and the drug-linker intermediate and calculation of the average number of conjugated drug molecules per antibody molecule of the antibody-drug conjugate can be performed with reference to descriptions in WO2014/057687, WO2015/098099, WO2015/115091, WO2015/155998, WO2017/002776, WO2018/212136, and so on.

In the present disclosure, the term “anti-HER2 antibody-drug conjugate” refers to an antibody-drug conjugate such that the antibody in the antibody-drug conjugate according to the present disclosure is an anti-HER2 antibody.

The anti-HER2 antibody is preferably an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1 and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2 and CDRL3 consisting of an amino acid sequence consisting of amino acid residues 89 to 97 of SEQ ID NO: 2, and more preferably an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2, and even more preferably an antibody comprising a heavy chain consisting of an amino acid sequence represented by SEQ ID NO: 1 and a light chain consisting of the amino acid sequence represented by SEQ ID NO: 2, or an antibody comprising a heavy chain consisting of amino acid residues 1 to 449 of SEQ ID NO: 1 and a light chain consisting of an amino acid sequence consisting of all amino acid residues 1 to 214 of SEQ ID NO: 2.

The average number of units of the drug-linker conjugated per antibody molecule in the anti-HER2 antibody-drug conjugate is preferably 2 to 8, more preferably 3 to 8, even more preferably 7 to 8, even more preferably 7.5 to 8, and even more preferably about 8.

A preferred anti-HER2 antibody-drug conjugate used in the present disclosure can be produced with reference to descriptions in WO2015/115091 and so on.

In preferred embodiments, the anti-HER2 antibody-drug conjugate is trastuzumab deruxtecan (DS-8201) or trastuzumab emtansine (T-DM1), preferably trastuzumab deruxtecan.

### 4. RASG12C Inhibitor

In the present disclosure, the term “RASG12C inhibitor” refers to a compound that inhibits a G12C mutant Ras protein, such as a protein encoded by KRAS, NRAS or HRAS gene, preferably KRAS gene. Preferred examples of RASG12C inhibitors can include those disclosed herein.

Examples of RASG12C inhibitors which may be used according to the present disclosure include compounds of formula (I) as disclosed in WO 2019/215203, which includes Compound A, and compounds disclosed in WO2019/099524, WO2020/178282, WO2021/118877 and WO2021/245051. Further examples include compounds designated as LY3537982 (Loxo/Lilly), AZD4625 (AstraZeneca), AMG510 (sotorasib: Amgen), MRTX849 (adagrasib: Mirati), JDQ443 (Novartis), GDC-6036 (Genentech), BI 1,823,911 (Boehringer Ingelheim), D1553 (InventisBio) and JNJ-7469917 (Johnson and Johnson) Preferably, the RASG12C inhibitor used has physicochemical properties that enable it to penetrate the blood brain barrier.

Accordingly, in preferred embodiments of the RASG12C inhibitor used in the present disclosure, the RASG12C inhibitor is a compound represented by the following formula (I):

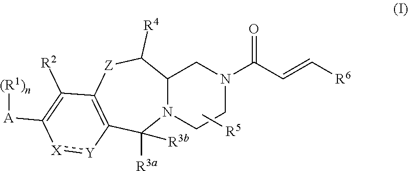

wherein:
A is phenyl or a bicyclic heteroaryl group;
X and Y are connected by a double bond and i) X is CR⁷ and Y is CR⁸, ii) X is N and Y is CR⁸, or iii) X is CR⁷ and Y is N; or
X and Y together are C(O)NR⁹; or
X and Y are adjacent ring atoms of an optionally substituted 5- or 6-membered N-heterocycle fused to the aromatic ring substituted with Z, and X and Y are both C or are C and N;
Z is O, NH, or NMe;
R¹ is independently selected from F, Cl, Br, OH, CH₂OH, OMe, CH₂OMe, C₁-C₃alkyl and C₁-C₃fluoroalkyl; n is 0, 1, 2 or 3;
R² is H, F, Cl, CCH, CCMe, CN, Br, C₁-C₃alkyl, C₁-C₃fluoroalkyl, OMe or OEt;
R^{3a} and R^{3b} together are ═O or R^{3a} and R^{3b} are H;
R⁴ is H or Me;
R⁵ is H or Me;
R⁶ is H or CH₂NMe₂;
R⁷ and R⁸ are independently selected from H, F, Cl, CCH, CC(C₁-C₃alkyl), CCCH₂NMe₂, CCCH₂O(C₁-C₃alkyl), CN, Me, C₁-C₆alkyl, OH, OMe, O(C₁-C₃alkyl), O(C₁-C₃deuteroalkyl), O(C₁-C₃fluoroalkyl), O(C₃-C₆cycloalkyl), C₁-C₃fluoroalkyl, OCH₂CH₂NMe₂, OCH₂CH₂OMe, CH₂OMe, OCH₂CH₂N(CH₂CH₂)₂CH, OCH₂CH₂N(CH₂CH₂)₂O, OCH₂CH₂ (2-pyridyl) or an optionally substituted 3-, 4-, 5- or 6-membered carbocycle or heterocycle; or
R⁷ and R⁸ combine to form an optionally substituted 5- or 6-membered carbocycle or heterocycle;
R⁹ is selected from H, Me, Et, C₃H₇ and C₁-C₃fluoroalkyl; or a pharmaceutically acceptable salt thereof.

In another embodiment, the RASG12C inhibitor is a compound of formula (I), in which i) X is CR⁷ and Y is CR⁸, ii) X is N and Y is CR⁸ or iii) X is CR⁷ and Y is N. In another embodiment the RASG12C inhibitor is a compound of formula (I) wherein Z is O. In another embodiment the RASG12C inhibitor is a compound of formula (I), wherein R^{3a} and R^{3b} are H. In another embodiment the RASG12C inhibitor is a compound of formula (I), wherein R⁴ is H. In another embodiment the RASG12C inhibitor is a compound of formula (I), wherein R⁶ is H. In another embodiment the RASG12C inhibitor is a compound of formula (I), wherein A is phenyl.

In another embodiment the RASG12C inhibitor is a compound of formula (I), selected from:
(12aS)-2-Acryloyl-10-chloro-9-(5-methyl-1H-indazol-4-yl)-1,2,3,4,12,12a-hexahydro-6H-benzo[f]pyrazino[2,1-c][1,4]oxazepin-6-one;
1-((12aS)-10-Chloro-9-(5-methyl-1H-indazol-4-yl)-3,4,12,12a-tetrahydro-6H-benzo[f]pyrazino[2,1-c][1,4]oxazepin-2(1H)-yl)prop-2-en-1-one;
1-[(12aR)-10-Chloro-9-(2-fluoro-6-hydroxyphenyl)-7-methoxy-3,4,12,12a-tetrahydro-6H-pyrazino[2,1-c][1,4]benzoxazepin-2(1H)-yl]prop-2-en-1-one;
(12aR)-10-chloro-9-(2-fluoro-6-hydroxyphenyl)-7-hydroxy-2-(prop-2-enoyl)-1,2,3,4,12,12a-hexahydro-6H-pyrazino[2,1-c][1,4]benzoxazepin-6-one;
(12aR)-10-Chloro-9-(5-methyl-1H-indazol-4-yl)-2-(prop-2-enoyl)-1,2,3,4,12,12a-hexahydro-6H-pyrazino[2,1-c][1,4]benzoxazepin-6-one;
1-((12aR)-10-Chloro-9-(5-methyl-1H-indazol-4-yl)-3,4,12,12a-tetrahydro-6H-benzo[f]pyrazino[2,1-c][1,4]oxazepin-2(1H)-yl)prop-2-en-1-one;
(12aR)-10-Chloro-8-fluoro-9-(2-fluoro-6-hydroxyphenyl)-2-(prop-2-enoyl)-1,2,3,4,12,12a-hexahydro-6H-pyrazino[2,1-c][1,4]benzoxazepin-6-one;
1-[(12aR)-8,10-dichloro-9-(2-fluoro-6-hydroxyphenyl)-7-hydroxy-3,4,12,12a-tetrahydro-6H-pyrazino[2,1-c][1,4]benzoxazepin-2(1H)-yl]prop-2-en-1-one;
1-[(12aR)-10-Chloro-9-(2-fluoro-6-hydroxyphenyl)-7-(1H-imidazol-1-yl)-3,4,12,12a-tetrahydro-6H-pyrazino[2,1-c][1,4]benzoxazepin-2(1H)-yl]prop-2-en-1-one;
(12aR)-10-Chloro-9-(2-fluoro-6-hydroxyphenyl)-2-(prop-2-enoyl)-1,2,3,4,12,12a-hexahydro-6H-pyrazino[2,1-c][1,4]benzoxazepine-7-carbonitrile;
1-[(12aR)-10-Chloro-9-(2-fluoro-6-hydroxyphenyl)-7-(1H-pyrazol-1-yl)-3,4,12,12a-tetrahydro-6H-pyrazino[2,1-c][1,4]benzoxazepin-2(1H)-yl]prop-2-en-1-one;
1-((12aR)-10-Chloro-8-fluoro-9-(5-methyl-1H-benzo[d]imidazol-4-yl)-3,4,12,12a-tetrahydro-6H-benzo[f]pyrazino[2,1-c][1,4]oxazepin-2(1H)-yl)prop-2-en-1-one;
(12aR)-8,10-Dichloro-9-(2-fluoro-6-hydroxyphenyl)-2-(prop-2-enoyl)-1,2,3,4,12,12a-hexahydro-6H-pyrazino[2,1-c][1,4]benzoxazepin-6-one;
(12aR)-10-Chloro-9-(2-fluoro-6-hydroxyphenyl)-2-(prop-2-enoyl)-1,2,3,4,12,12a-hexahydro-6H-pyrazino[2,1-c][1,4]benzoxazepine-8-carbonitrile;
(12aR)-10-Chloro-9-(2-fluoro-6-hydroxyphenyl)-8-methyl-2-(prop-2-enoyl)-1,2,3,4,12,12a-hexahydro-6H-pyrazino[2,1-c][1,4]benzoxazepin-6-one;
1-[(12aR)-8,10-Dichloro-9-(2-fluoro-6-hydroxyphenyl)-3,4,12,12a-tetrahydro-6H-pyrazino[2,1-c][1,4]benzoxazepin-2(1H)-yl]prop-2-en-1-one;
8-[(12aR)-10-Chloro-8-fluoro-2-(prop-2-enoyl)-1,2,3,4,12,12a-hexahydro-6H-pyrazino[2,1-c][1,4]benzoxazepin-9-yl]-7-methylisoquinolin-1(2H)-one;
1-[(12aR)-10-chloro-9-(2-fluoro-6-hydroxyphenyl)-8-methoxy-3,4,12,12a-tetrahydro-6H-pyrazino[2,1-c][1,4]benzoxazepin-2(1H)-yl]prop-2-en-1-one;
(12aS)-10-Chloro-9-(5-methyl-1H-indazol-4-yl)-2-(prop-2-enoyl)-1,3,4,11,12,12a-hexahydropyrazino[2,1-c][1,4]benzodiazepin-6(2H)-one;
1-[(12aR)-10-chloro-9-(2-chloro-6-hydroxyphenyl)-8-fluoro-3,4,12,12a-tetrahydro-6H-pyrazino[2,1-c][1,4]benzoxazepin-2(1H)-yl]prop-2-en-1-one;
(12aS)-10-Chloro-11-methyl-9-(5-methyl-1H-indazol-4-yl)-2-(prop-2-enoyl)-1,3,4,11,12,12a-hexahydropyrazino[2,1-c][1,4]benzodiazepin-6(2H)-one;
1-[(12aR)-10-Chloro-9-(2,3-difluoro-6-hydroxyphenyl)-8-fluoro-3,4,12,12a-tetrahydro-6H-pyrazino[2,1-c][1,4]benzoxazepin-2(1H)-yl]prop-2-en-1-one;
(12aR)-10-Chloro-9-(2-hydroxy-6-methylphenyl)-2-(prop-2-enoyl)-1,2,3,4,12,12a-hexahydro-6H-pyrazino[2,1-c][1,4]benzoxazepine-8-carbonitrile;
1-[(12aR)-9-(2-Chloro-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-8,10-Difluoro-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-8,10-Difluoro-9-[2-fluoro-6-(hydroxymethyl)phenyl]-3,4,12,12a-tetrahydro-6H-pyrazino[2,1-c][1,4]benzoxazepin-2(1H)-yl]prop-2-en-1-one;
1-[(12aR)-8,10-Difluoro-9-[2-hydroxy-6-(trifluoromethyl)phenyl]-3,4,12,12a-tetrahydro-6H-pyrazino[2,1-c][1,4]benzoxazepin-2(1H)-yl]prop-2-en-1-one;
1-[(12aR)-9-(2-Ethyl-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-9-[2-(Difluoromethyl)-6-hydroxyphenyl]-8,10-difluoro-3,4,12,12a-tetrahydro-6H-pyrazino[2,1-c][1,4]benzoxazepin-2(1H)-yl]prop-2-en-1-one;
(12aR)-9-(2-Chloro-6-hydroxyphenyl)-10-fluoro-2-(prop-2-enoyl)-1,2,3,4,12,12a-hexahydro-6H-pyrazino[2,1-c][1,4]benzoxazepine-8-carbonitrile; (12aR)-10-Chloro-9-(2-chloro-6-hydroxyphenyl)-2-(prop-2-enoyl)-1,2,3,4,12,12a-hexahydro-6H-pyrazino[2,1-c][1,4]benzoxazepine-8-carbonitrile;
1-[(12aR)-9-(2-Bromo-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-8-Chloro-10-fluoro-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-8-Chloro-10-ethynyl-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-10-Ethynyl-8-fluoro-9-(2-hydroxy-6-methylphenyl)-3,4,12,12a-tetrahydro-6H-pyrazino[2,1-c][1,4]benzoxazepin-2(1H)-yl]prop-2-en-1-one;
(6aR)-4-Chloro-3-(2-fluoro-6-hydroxyphenyl)-2-methyl-8-(prop-2-enoyl)-2,6,6a,7,8,9,10,12-octahydro-1H-pyrazino[2,1-c]pyrido[3,4-f][1,4]oxazepin-1-one;
1-[(6aR)-1,4-Dichloro-3-(2-fluoro-6-hydroxyphenyl)-6a,7,9,10-tetrahydro-12H-pyrazino[2,1-c]pyrido[3,4-f][1,4]oxazepin-8(6H)-yl]prop-2-en-1-one;
(6aR)-4-Chloro-3-(2-fluoro-6-hydroxyphenyl)-8-(prop-2-enoyl)-2,6,6a,7,8,9,10,12-octahydro-1H-pyrazino[2,1-c]pyrido[3,4-f][1,4]oxazepin-1-one;
1-[(8aR)-6-Chloro-5-(2-fluoro-6-hydroxyphenyl)-8a,9,11,12-tetrahydro-14H-pyrazino[2,1-c][1,2,4]triazolo[4′,3′:1,2]pyrido[3,4-f][1,4]oxazepin-10(8H)-yl]prop-2-en-1-one;
1-[(7aR)-5-chloro-4-(2-fluoro-6-hydroxyphenyl)-1-methyl-1,7a,8,10,11,13-hexahydropyrazino[2′,1′:3,4][1,4]oxazepino[7,6-g]indazol-9(7H)-yl]prop-2-en-1-one;
1-[(7aR)-5-Chloro-4-(2-fluoro-6-hydroxyphenyl)-2-methyl-2,7a,8,10,11,13-hexahydropyrazino[2′,1′:3,4][1,4]oxazepino[7,6-g]indazol-9(7H)-yl]prop-2-en-1-one;
1-[(12aR)-9-(2-chloro-6-hydroxyphenyl)-8-ethynyl-10-fluoro-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-ethynyl-3,4,12,12a-tetrahydro-6H-pyrazino[2,1-c][1,4]benzoxazepin-2(1H)-yl]prop-2-en-1-one;
1-((12aR)-10-chloro-8-ethynyl-9-(2-fluoro-6-hydroxyphenyl)-3,4,12,12a-tetrahydro-6H-benzo[f]pyrazino[2,1-c][1,4]oxazepin-2(1H)-yl)prop-2-en-1-one;
1-[(7aR)-5-Chloro-4-(2-chloro-6-hydroxyphenyl)-1-methyl-1,7a,8,10,11,13-hexahydroimidazo[4,5-g]pyrazino[2,1-c][1,4]benzoxazepin-9(7H)-yl]prop-2-en-1-one;
1-[(12aR)-8-Chloro-9-(2-chloro-6-hydroxyphenyl)-10-fluoro-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-10-fluoro-8-(prop-1-yn-1-yl)-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-2-ethynyl-6a,7,9,10-tetrahydro-12H-pyrazino[2,1-c]pyrido[2,3-f][1,4]oxazepin-8(6H)-yl]prop-2-en-1-one;
1-[(12aR)-9-(2-Chloro-6-hydroxyphenyl)-8-ethynyl-10-methyl-3,4,12,12a-tetrahydro-6H-pyrazino[2,1-c][1,4]benzoxazepin-2(1H)-yl]prop-2-en-1-one;
1-[(12aR)-10-chloro-9-(2-chloro-6-hydroxyphenyl)-7,8-difluoro-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-(difluoromethoxy)-10-fluoro-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-ethynyl-7,10-difluoro-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-(difluoromethoxy)-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-(cyclopropyloxy)-10-fluoro-3,4,12,12a-tetrahydro-6H-pyrazino[2,1-c][1,4]benzoxazepin-2(1H)-yl]prop-2-en-1-one;
1-((12aR)-9-(2-Chloro-6-hydroxyphenyl)-8-(3-(dimethylamino)prop-1-yn-1-yl)-10-fluoro-3,4,12,12a-tetrahydro-6H-benzo[f]pyrazino[2,1-c][1,4]oxazepin-2(1H)-yl)prop-2-en-1-one;
1-[(12aR)-9-(2-chloro-6-hydroxyphenyl)-10-fluoro-8-[(pyridin-4-yl)methoxy]-3,4,12,12a-tetrahydro-6H-pyrazino[2,1-c][1,4]benzoxazepin-2(1H)-yl]prop-2-en-1-one;
1-[(12aR)-10-chloro-9-(2-chloro-6-hydroxyphenyl)-8-(2-methoxyethoxy)-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-10-fluoro-8-[2-(piperidin-1-yl)ethoxy]-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-(prop-1-yn-1-yl)-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-2-[(<2>H3)methyloxy]-6a,7,9,10-tetrahydro-12H-pyrazino[2,1-c]pyrido[2,3-f][1,4]oxazepin-8(6H)-yl]prop-2-en-1-one;
1-[(12aR)-10-Chloro-9-(2-chloro-6-hydroxyphenyl)-8-(methoxymethyl)-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-7-[2-(dimethylamino)ethoxy]-10-fluoro-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-1-(prop-1-yn-1-yl)-6a,7,9,10-tetrahydro-12H-pyrazino[2,1-c]pyrido[3,4-f][1,4]oxazepin-8(6H)-yl]prop-2-en-1-one; and
1-((6aR)-4-Chloro-3-(2-chloro-6-hydroxyphenyl)-1-ethynyl-6a,7,9,10-tetrahydro-12H-pyrazino[2,1-c]pyrido[3,4-f][1,4]oxazepin-8(6H)-yl)prop-2-en-1-one; or a pharmaceutically acceptable salt thereof.

In another embodiment the RASG12C inhibitor is a compound of formula (I), which is 1-[(12aR)-9-(2-chloro-6-hydroxyphenyl)-8-ethynyl-10-fluoro-3,4,12,12a-tetrahydro-6H-pyrazino[2,1-c][1,4]benzoxazepin-2(1H)-yl]prop-2-en-1-one.

In another embodiment the RASG12C inhibitor is a compound of formula (I), which is 1-[(12aR)-9-(2-chloro-6-hydroxyphenyl)-10-fluoro-8-(prop-1-yn-1-yl)-3,4,12,12a-tetrahydro-6H-pyrazino[2,1-c][1,4]benzoxazepin-2(1H)-yl]prop-2-en-1-one.

In other preferred embodiments of the RASG12C inhibitor used in the present disclosure, the RASG12C inhibitor is a compound represented by the following formula (II):

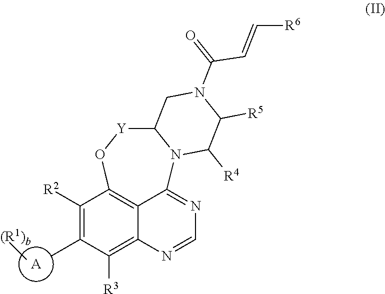

wherein:
Ring A is selected from phenyl and bicyclic heteroaryl;
R¹ in each occurrence is independently selected from C₁₋₄alkyl, halo, hydroxy, C₁₋₄alkoxy, C₁₋₃fluoroalkyl, C₁₋₃fluoroalkoxy, cyano and acetylenyl;
b is 0, 1, 2 or 3;
Y is CH₂ or CH₂CH₂;
R² is cyano, halo, C₁₋₄alkyl, C₁₋₄alkoxy or C₁₋₃fluoroalkyl;
R³ is F, Me, Et, MeO or C₁₋₂fluoroalkyl;
R⁴ is H or Me;
R⁵ is H or Me;
R⁶ is H or CH₂NMe₂;
or a pharmaceutically acceptable salt thereof, provided that when Y is CH₂, R² is Cl, R³ is F, A is phenyl,
b is 2, the groups R¹ are F and OH and are each ortho to the biaryl bond, and when both R⁴ and R⁶ are H, then R⁵ is Me.

In one embodiment the RASG12C inhibitor is a compound of formula (II), having the structure:

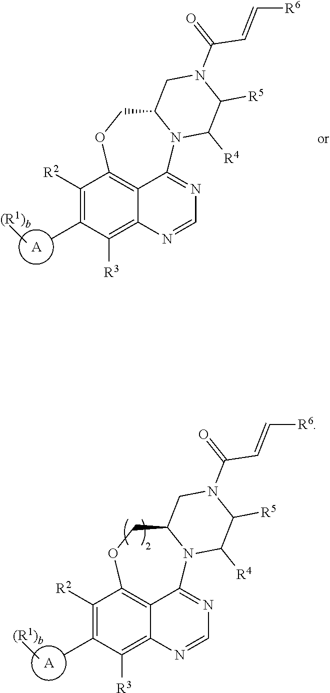

In another embodiment the RASG12C inhibitor is a compound of formula (II) wherein the Ring A is selected from the group consisting of:

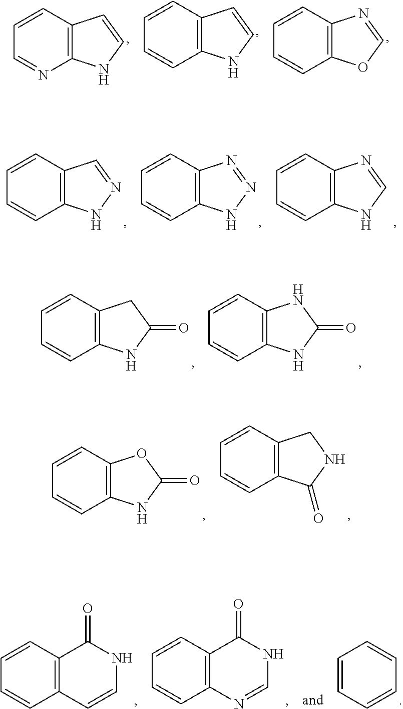

In another embodiment the RASG12C inhibitor is a compound of formula (II) wherein R⁴ is H. In another embodiment the RASG12C inhibitor is a compound of formula (II) wherein R⁶ is H. In another embodiment the RASG12C inhibitor is a compound of formula (II) wherein Y is CH₂. In another embodiment the RASG12C inhibitor is a compound of formula (II) wherein Y is CH₂CH₂. In another embodiment the RASG12C inhibitor is a compound of formula (II) wherein R² is Cl. In another embodiment the RASG12C inhibitor is a compound of formula (II) wherein R³ is F. In another embodiment the RASG12C inhibitor is a compound of formula (II) wherein R⁴ is H and R⁵ is Me.

In another embodiment the RASG12C inhibitor is a compound of formula (II) selected from:
7-[(8aS)-10-Acryloyl-6-chloro-4-fluoro-8,8a,9,10,11,12-hexahydropyrazino[2′,1′:3,4][1,4]oxazepino[5,6,7-de]quinazolin-5-yl]-6-methyl-2,3-dihydro-1H-isoindol-1-one;
1-[(8aS,11S)-6-Chloro-4-fluoro-5-(2-fluoro-6-hydroxyphenyl)-11-methyl-8a,9,11,12-tetrahydropyrazino[2′,1′:3,4][1,4]oxazepino[5,6,7-de]quinazolin-10(8H)-yl]prop-2-en-1-one;
1-[(8aS,11R)-6-Chloro-4-fluoro-5-(2-fluoro-6-hydroxyphenyl)-11-methyl-8a,9,11,12-tetrahydropyrazino[2′,1′:3,4][1,4]oxazepino[5,6,7-de]quinazolin-10(8H)-yl]prop-2-en-1-one;
5-[(8aS)-10-Acryloyl-6-chloro-4-fluoro-8,8a,9,10,11,12-hexa hydropyrazino[2′,1′:3,4][1,4]oxazepino[5,6,7-de]quinazolin-5-yl]-6-methylquinazolin-4(3H)-one;
1-[(8aS)-6-Chloro-4-fluoro-5-(5-methyl-1H-benzimidazol-4-yl)-8a,9,11,12-tetrahydropyrazino[2′,1′:3,4][1,4]oxazepino[5,6,7-de]quinazolin-10(8H)-yl]prop-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]isoquinolin-1(2H)-one;
1-[(8aS)-6-Chloro-4-fluoro-5-(1H-indazol-3-yl)-8a,9,11,12-tetrahydropyrazino[2′,1′:3,4][1,4]oxazepino[5,6,7-de]quinazolin-10(8H)-yl]prop-2-en-1-one;
1-[(8aS)-6-Chloro-4-fluoro-5-(2-hydroxy-6-methylphenyl)-8a,9,11,12-tetrahydropyrazino[2′,1′:3,4][1,4]oxazepino[5,6,7-de]quinazolin-10(8H)-yl]prop-2-en-1-one;
(2E)-1-[(8aS)-6-Chloro-4-fluoro-5-(2-fluoro-6-hydroxyphenyl)-8a,9,11,12-tetrahydropyrazino[2′,1′:3,4][1,4]oxazepino[5,6,7-de]quinazolin-10(8H)-yl]-4-(dimethylamino)but-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]-7-methylisoquinolin-1(2H)-one;
1-[(8aS)-6-Chloro-4-fluoro-5-(5-methyl-1H-benzotriazol-4-yl)-8a,9,11,12-tetrahydropyrazino[2′,1′:3,4][1,4]oxazepino[5,6,7-de]quinazolin-10(8H)-yl]prop-2-en-1-one;
1-((8aS)-6-chloro-4-fluoro-5-(5-fluoro-1H-benzo[d]imidazol-4-yl)-8a,9,11,12-tetrahydropyrazino[2′,1′:3,4][1,4]oxazepino[5,6,7-de]quinazolin-10(8H)-yl)prop-2-en-1-one;
1-[(8aS)-6-Chloro-4-fluoro-5-(5-fluoro-1H-indazol-4-yl)-8a,9,11,12-tetrahydropyrazino[2′,1′:3,4][1,4]oxazepino[5,6,7-de]quinazolin-10(8H)-yl]prop-2-en-1-one;
1-((8aS)-6-Chloro-4-fluoro-5-(5-fluoro-1-methyl-1H-benzo[d]imidazol-4-yl)-8a,9,11,12-tetrahydropyrazino[2′,1′:3,4][1,4]oxazepino[5,6,7-de]quinazolin-10(8H)-yl)prop-2-en-1-one;
1-[(8aS)-6-Chloro-4-fluoro-5-(5-fluoro-1H-benzotriazol-4-yl)-8a,9,11,12-tetrahydropyrazino[2′,1′:3,4][1,4]oxazepino[5,6,7-de]quinazolin-10(8H)-yl]prop-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]-7-fluoroisoquinolin-1(2H)-one;
(2E)-1-[(8aS)-6-chloro-4-fluoro-5-(5-methyl-1H-indazol-4-yl)-8a,9,11,12-tetrahydropyrazino[2′,1′:3,4][1,4]oxazepino[5,6,7-de]quinazolin-10(8H)-yl]-4-(dimethylamino)but-2-en-1-one;
1-[(6aR,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aR,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
8-[3-Chloro-1-fluoro-8-(prop-2-enoyl)-6,6a,7,8,9,10-hexahydro-5H-pyrazino[1′,2′:5,6][1,5]oxazocino[4,3,2-de]quinazolin-2-yl]-7-methylisoquinolin-1(2H)-one;
1-[(6aS,9R)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aR,9R)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aS,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(8aS)-4-Chloro-6-fluoro-5-(2-fluoro-6-hydroxyphenyl)-8a,9,11,12-tetrahydropyrazino[2′,1′:3,4][1,4]oxazepino[5,6,7-de]quinazolin-10(8H)-yl]prop-2-en-1-one;
1-[(8aS,11R)-6-Chloro-4-fluoro-11-methyl-5-(5-methyl-1H-benzimidazol-4-yl)-8a,9,11,12-tetrahydropyrazino[2′,1′:3,4][1,4]oxazepino[5,6,7-de]quinazolin-10(8H)-yl]prop-2-en-1-one;
8-[(8aS,11R)-6-Chloro-4-fluoro-11-methyl-10-(prop-2-enoyl)-8,8a,9,10,11,12-hexahydropyrazino[2′,1′:3,4][1,4]oxazepino[5,6,7-de]quinazolin-5-yl]-7-methylisoquinolin-1(2H)-one;
(2E)-1-[(8aS,11R)-6-Chloro-4-fluoro-11-methyl-5-(5-methyl-1H-benzimidazol-4-yl)-8a,9,11,12-tetrahydropyrazino[2′,1′:3,4][1,4]oxazepino[5,6,7-de]quinazolin-10(8H)-yl]-4-(dimethylamino)but-2-en-1-one; and
(2E)-1-[(8aS,11R)-6-Chloro-4-fluoro-11-methyl-5-(5-methyl-1H-indazol-4-yl)-8a,9,11,12-tetrahydropyrazino[2′,1′:3,4][1,4]oxazepino[5,6,7-de]quinazolin-10(8H)-yl]-4-(dimethylamino)but-2-en-1-one;
   or a pharmaceutically acceptable salt thereof.

In a particularly preferred embodiment the RASG12C inhibitor used in the disclosure is the Compound A (1-[(6aS,9R)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one) represented by the following formula:

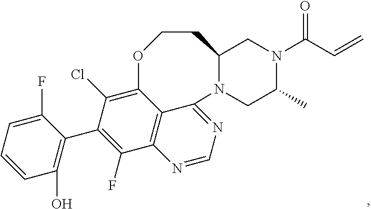 [SMILES: C=CC(=O)N1C[C@@H]2CCOc3c(Cl)c(-c4c(O)cccc4F)c(F)c4ncnc(c34)N2C[C@H]1C]

or a pharmaceutically acceptable salt thereof.

In another preferred embodiment the RASG12C inhibitor used in the disclosure is AMG510 (sotorasib: Amgen).

In another preferred embodiment the RASG12C inhibitor used in the disclosure is MRTX849 (adagrasib: Mirati).

In another embodiment the RASG12C inhibitor used in the disclosure is 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-7-fluoro-benzothiophene-3-carbonitrile.

### 5. Combination of Antibody-Drug Conjugate and RASG12C Inhibitor

In a first combination embodiment of the disclosure, the antibody-drug conjugate which is combined with the RASG12C inhibitor is an antibody-drug conjugate in which the antibody is an anti-HER2 antibody.

In an embodiment of the first combination embodiment described above, the antibody-drug conjugate which is combined with the RASG12C inhibitor is an antibody-drug conjugate wherein the anti-HER2 antibody is an antibody comprising a heavy chain comprising CDRH1 consisting of an amino acid sequence represented by SEQ ID NO: 3, CDRH2 consisting of an amino acid sequence represented by SEQ ID NO: 4 and CDRH3 consisting of an amino acid sequence represented by SEQ ID NO: 5, and a light chain comprising CDRL1 consisting of an amino acid sequence represented by SEQ ID NO: 6, CDRL2 consisting of an amino acid sequence consisting of amino acid residues 1 to 3 of SEQ ID NO: 7 and CDRL3 consisting of an amino acid sequence represented by SEQ ID NO: 8.

In another embodiment of the first combination embodiment described above, the anti-HER2 antibody is an antibody comprising a heavy chain comprising a heavy chain variable region consisting of an amino acid sequence represented by SEQ ID NO: 9 and a light chain comprising a light chain variable region consisting of an amino acid sequence represented by SEQ ID NO: 10.

In another embodiment of the first combination embodiment described above, the anti-HER2 antibody is an antibody comprising a heavy chain consisting of an amino acid sequence represented by SEQ ID NO: 1 and a light chain consisting of an amino acid sequence represented by SEQ ID NO: 2.

In another embodiment of the first combination embodiment described above, the anti-HER2 antibody is an antibody comprising a heavy chain consisting of an amino acid sequence represented by SEQ ID NO: 11 and a light chain consisting of an amino acid sequence represented by SEQ ID NO: 2.

In another embodiment of the first combination embodiment described above, the antibody-drug conjugate is an antibody-drug conjugate in which a drug-linker represented by the following formula:

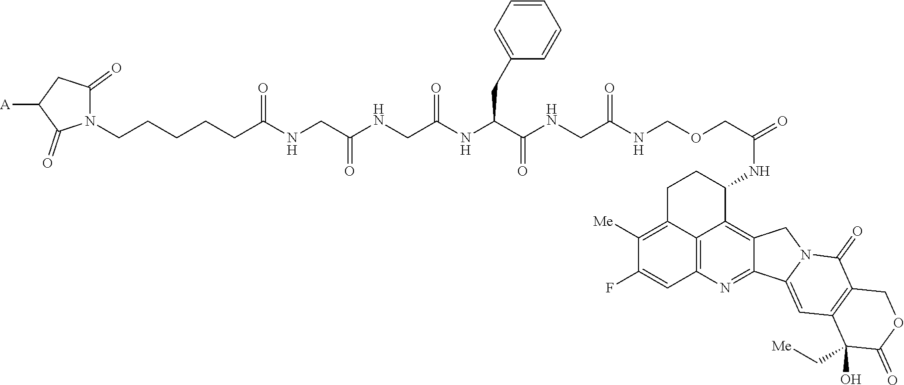

wherein A represents the connecting position to an antibody, is conjugated to an anti-HER2 antibody via a thioether bond.

In another combination embodiment of the disclosure, the antibody-drug conjugate which is combined with the RASG12C inhibitor is trastuzumab deruxtecan (DS-8201) or trastuzumab emtansine (T-DM1).

In an embodiment of each of the combination embodiments described above, the antibody-drug conjugate as defined for the above combination embodiments is combined with a RASG12C inhibitor which is a compound represented by the following formula (I):

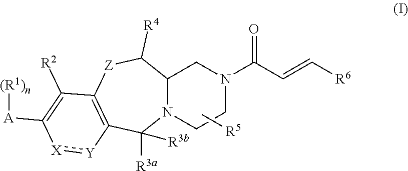

wherein:
A is phenyl or a bicyclic heteroaryl group;
X and Y are connected by a double bond and i) X is CR⁷ and Y is CR⁸, ii) X is N and Y is CR⁸, or iii) X is CR⁷ and Y is N; or X and Y together are C(O)NR⁹; or
X and Y are adjacent ring atoms of an optionally substituted 5- or 6-membered N-heterocycle fused to the aromatic ring substituted with Z, and X and Y are both C or are C and N;
Z is O, NH, or NMe;
R¹ is independently selected from F, Cl, Br, OH, CH₂OH, OMe, CH₂OMe, C₁-C₃alkyl and C₁-C₃fluoroalkyl; n is 0, 1, 2 or 3; R² is H, F, Cl, CCH, CCMe, CN, Br, C₁-C₃alkyl, C₁-C₃fluoroalkyl, OMe or OEt;
R^{3a} and R^{3b} together are ═O or R^{3a} and R^{3b} are H;
R⁴ is H or Me;
R⁵ is H or Me;
R⁶ is H or CH₂NMe₂;
R⁷ and R⁸ are independently selected from H, F, Cl, CCH, CC(C₁-C₃alkyl), CCCH₂NMe₂, CCCH₂O(C₁-C₃alkyl), CN, Me, C₁-C₆alkyl, OH, OMe, O(C₁-C₃alkyl), O(C₁-C₃deuteroalkyl), O(C₁-C₃fluoroalkyl), O(C₃-C₆cycloalkyl), C₁-C₃fluoroalkyl, OCH₂CH₂NMe₂, OCH₂CH₂OMe, CH₂OMe, OCH₂CH₂N(CH₂CH₂)₂CH, OCH₂CH₂N(CH₂CH₂) 20, OCH₂CH₂ (2-pyridyl) or an optionally substituted 3-, 4-, 5- or 6-membered carbocycle or heterocycle; or
R⁷ and R⁸ combine to form an optionally substituted 5- or 6-membered carbocycle or heterocycle;
R⁹ is selected from H, Me, Et, C₃H₇ and C₁-C₃fluoroalkyl; or a pharmaceutically acceptable salt thereof.

In another combination embodiment, the antibody-drug conjugate as defined for the above combination embodiments is combined with a RASG12C inhibitor as defined above wherein, in formula (I), i) X is CR⁷ and Y is CR⁸, ii) X is N and Y is CR⁸ or iii) X is CR⁷ and Y is N. In another embodiment, in formula (I), Z is 0. In another embodiment, in formula (I), R^{3a} and R^{3b} are H. In another embodiment, in formula (I), R⁴ is H. In another embodiment, in formula (I), R⁶ is H. In another embodiment, in formula (I), A is phenyl.

In another combination embodiment, the anti-HER2 antibody-drug conjugate as defined for the above combination embodiments is combined with a RASG12C inhibitor wherein the RASG12C inhibitor is a compound of formula (I) selected from:
(12aS)-2-Acryloyl-10-chloro-9-(5-methyl-1H-indazol-4-yl)-1,2,3,4,12,12a-hexahydro-6H-benzo[f]pyrazino[2,1-c][1,4]oxazepin-6-one;
1-((12aS)-10-Chloro-9-(5-methyl-1H-indazol-4-yl)-3,4,12,12a-tetrahydro-6H-benzo[f]pyrazino[2,1-c][1,4]oxazepin-2(1H)-yl)prop-2-en-1-one;
1-[(12aR)-10-Chloro-9-(2-fluoro-6-hydroxyphenyl)-7-methoxy-3,4,12,12a-tetrahydro-6H-pyrazino[2,1-c][1,4]benzoxazepin-2(1H)-yl]prop-2-en-1-one;
(12aR)-10-chloro-9-(2-fluoro-6-hydroxyphenyl)-7-hydroxy-2-(prop-2-enoyl)-1,2,3,4,12,12a-hexahydro-6H-pyrazino[2,1-c][1,4]benzoxazepin-6-one;
(12aR)-10-Chloro-9-(5-methyl-1H-indazol-4-yl)-2-(prop-2-enoyl)-1,2,3,4,12,12a-hexahydro-6H-pyrazino[2,1-c][1,4]benzoxazepin-6-one;
1-((12aR)-10-Chloro-9-(5-methyl-1H-indazol-4-yl)-3,4,12,12a-tetrahydro-6H-benzo[f]pyrazino[2,1-c][1,4]oxazepin-2(1H)-yl)prop-2-en-1-one;
(12aR)-10-Chloro-8-fluoro-9-(2-fluoro-6-hydroxyphenyl)-2-(prop-2-enoyl)-1,2,3,4,12,12a-hexahydro-6H-pyrazino[2,1-c][1,4]benzoxazepin-6-one;
1-[(12aR)-8,10-dichloro-9-(2-fluoro-6-hydroxyphenyl)-7-hydroxy-3,4,12,12a-tetrahydro-6H-pyrazino[2,1-c][1,4]benzoxazepin-2(1H)-yl]prop-2-en-1-one;
1-[(12aR)-10-Chloro-9-(2-fluoro-6-hydroxyphenyl)-7-(1H-imidazol-1-yl)-3,4,12,12a-tetrahydro-6H-pyrazino[2,1-c][1,4]benzoxazepin-2(1H)-yl]prop-2-en-1-one;
(12aR)-10-Chloro-9-(2-fluoro-6-hydroxyphenyl)-2-(prop-2-enoyl)-1,2,3,4,12,12a-hexahydro-6H-pyrazino[2,1-c][1,4]benzoxazepine-7-carbonitrile;
1-[(12aR)-10-Chloro-9-(2-fluoro-6-hydroxyphenyl)-7-(1H-pyrazol-1-yl)-3,4,12,12a-tetrahydro-6H-pyrazino[2,1-c][1,4]benzoxazepin-2(1H)-yl]prop-2-en-1-one;
1-((12aR)-10-Chloro-8-fluoro-9-(5-methyl-1H-benzo[d]imidazol-4-yl)-3,4,12,12a-tetrahydro-6H-benzo[f]pyrazino[2,1-c][1,4]oxazepin-2(1H)-yl)prop-2-en-1-one;
(12aR)-8,10-Dichloro-9-(2-fluoro-6-hydroxyphenyl)-2-(prop-2-enoyl)-1,2,3,4,12,12a-hexahydro-6H-pyrazino[2,1-c][1,4]benzoxazepin-6-one;
(12aR)-10-Chloro-9-(2-fluoro-6-hydroxyphenyl)-2-(prop-2-enoyl)-1,2,3,4,12,12a-hexahydro-6H-pyrazino[2,1-c][1,4]benzoxazepine-8-carbonitrile;
(12aR)-10-Chloro-9-(2-fluoro-6-hydroxyphenyl)-8-methyl-2-(prop-2-enoyl)-1,2,3,4,12,12a-hexahydro-6H-pyrazino[2,1-c][1,4]benzoxazepin-6-one;
1-[(12aR)-8,10-Dichloro-9-(2-fluoro-6-hydroxyphenyl)-3,4,12,12a-tetrahydro-6H-pyrazino[2,1-c][1,4]benzoxazepin-2(1H)-yl]prop-2-en-1-one;
8-[(12aR)-10-Chloro-8-fluoro-2-(prop-2-enoyl)-1,2,3,4,12,12a-hexahydro-6H-pyrazino[2,1-c][1,4]benzoxazepin-9-yl]-7-methylisoquinolin-1(2H)-one;
1-[(12aR)-10-chloro-9-(2-fluoro-6-hydroxyphenyl)-8-methoxy-3,4,12,12a-tetrahydro-6H-pyrazino[2,1-c][1,4]benzoxazepin-2(1H)-yl]prop-2-en-1-one;
(12aS)-10-Chloro-9-(5-methyl-1H-indazol-4-yl)-2-(prop-2-enoyl)-1,3,4,11,12,12a-hexahydropyrazino[2,1-c][1,4]benzodiazepin-6(2H)-one;
1-[(12aR)-10-chloro-9-(2-chloro-6-hydroxyphenyl)-8-fluoro-3,4,12,12a-tetrahydro-6H-pyrazino[2,1-c][1,4]benzoxazepin-2(1H)-yl]prop-2-en-1-one;
(12aS)-10-Chloro-11-methyl-9-(5-methyl-1H-indazol-4-yl)-2-(prop-2-enoyl)-1,3,4,11,12,12a-hexahydropyrazino[2,1-c][1,4]benzodiazepin-6(2H)-one;
1-[(12aR)-10-Chloro-9-(2,3-difluoro-6-hydroxyphenyl)-8-fluoro-3,4,12,12a-tetrahydro-6H-pyrazino[2,1-c][1,4]benzoxazepin-2(1H)-yl]prop-2-en-1-one;
(12aR)-10-Chloro-9-(2-hydroxy-6-methylphenyl)-2-(prop-2-enoyl)-1,2,3,4,12,12a-hexahydro-6H-pyrazino[2,1-c][1,4]benzoxazepine-8-carbonitrile;
1-[(12aR)-9-(2-Chloro-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-8,10-Difluoro-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-8,10-Difluoro-9-[2-fluoro-6-(hydroxymethyl)phenyl]-3,4,12,12a-tetrahydro-6H-pyrazino[2,1-c][1,4]benzoxazepin-2(1H)-yl]prop-2-en-1-one;
1-[(12aR)-8,10-Difluoro-9-[2-hydroxy-6-(trifluoromethyl)phenyl]-3,4,12,12a-tetrahydro-6H-pyrazino[2,1-c][1,4]benzoxazepin-2(1H)-yl]prop-2-en-1-one;
1-[(12aR)-9-(2-Ethyl-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-9-[2-(Difluoromethyl)-6-hydroxyphenyl]-8,10-difluoro-3,4,12,12a-tetrahydro-6H-pyrazino[2,1-c][1,4]benzoxazepin-2(1H)-yl]prop-2-en-1-one;
(12aR)-9-(2-Chloro-6-hydroxyphenyl)-10-fluoro-2-(prop-2-enoyl)-1,2,3,4,12,12a-hexahydro-6H-pyrazino[2,1-c][1,4]benzoxazepine-8-carbonitrile; (12aR)-10-Chloro-9-(2-chloro-6-hydroxyphenyl)-2-(prop-2-enoyl)-1,2,3,4,12,12a-hexahydro-6H-pyrazino[2,1-c][1,4]benzoxazepine-8-carbonitrile;
1-[(12aR)-9-(2-Bromo-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-8-Chloro-10-fluoro-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-8-Chloro-10-ethynyl-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-10-Ethynyl-8-fluoro-9-(2-hydroxy-6-methylphenyl)-3,4,12,12a-tetrahydro-6H-pyrazino[2,1-c][1,4]benzoxazepin-2(1H)-yl]prop-2-en-1-one;
(6aR)-4-Chloro-3-(2-fluoro-6-hydroxyphenyl)-2-methyl-8-(prop-2-enoyl)-2,6,6a,7,8,9,10,12-octahydro-1H-pyrazino[2,1-c]pyrido[3,4-f][1,4]oxazepin-1-one;
1-[(6aR)-1,4-Dichloro-3-(2-fluoro-6-hydroxyphenyl)-6a,7,9,10-tetrahydro-12H-pyrazino[2,1-c]pyrido[3,4-f][1,4]oxazepin-8(6H)-yl]prop-2-en-1-one;
(6aR)-4-Chloro-3-(2-fluoro-6-hydroxyphenyl)-8-(prop-2-enoyl)-2,6,6a,7,8,9,10,12-octahydro-1H-pyrazino[2,1-c]pyrido[3,4-f][1,4]oxazepin-1-one;
1-[(8aR)-6-Chloro-5-(2-fluoro-6-hydroxyphenyl)-8a,9,11,12-tetrahydro-14H-pyrazino[2,1-c][1,2,4]triazolo[4′,3′:1,2]pyrido[3,4-f][1,4]oxazepin-10(8H)-yl]prop-2-en-1-one;
1-[(7aR)-5-chloro-4-(2-fluoro-6-hydroxyphenyl)-1-methyl-1,7a,8,10,11,13-hexahydropyrazino[2′,1′:3,4][1,4]oxazepino[7,6-g]indazol-9(7H)-yl]prop-2-en-1-one;
1-[(7aR)-5-Chloro-4-(2-fluoro-6-hydroxyphenyl)-2-methyl-2,7a,8,10,11,13-hexahydropyrazino[2′,1′:3,4][1,4]oxazepino[7,6-g]indazol-9(7H)-yl]prop-2-en-1-one;
1-[(12aR)-9-(2-chloro-6-hydroxyphenyl)-8-ethynyl-10-fluoro-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-ethynyl-3,4,12,12a-tetrahydro-6H-pyrazino[2,1-c][1,4]benzoxazepin-2(1H)-yl]prop-2-en-1-one;
1-((12aR)-10-chloro-8-ethynyl-9-(2-fluoro-6-hydroxyphenyl)-3,4,12,12a-tetrahydro-6H-benzo[f]pyrazino[2,1-c][1,4]oxazepin-2(1H)-yl)prop-2-en-1-one;
1-[(7aR)-5-Chloro-4-(2-chloro-6-hydroxyphenyl)-1-methyl-1,7a,8,10,11,13-hexahydroimidazo[4,5-g]pyrazino[2,1-c][1,4]benzoxazepin-9(7H)-yl]prop-2-en-1-one;
1-[(12aR)-8-Chloro-9-(2-chloro-6-hydroxyphenyl)-10-fluoro-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-10-fluoro-8-(prop-1-yn-1-yl)-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-2-ethynyl-6a,7,9,10-tetrahydro-12H-pyrazino[2,1-c]pyrido[2,3-f][1,4]oxazepin-8(6H)-yl]prop-2-en-1-one;
1-[(12aR)-9-(2-Chloro-6-hydroxyphenyl)-8-ethynyl-10-methyl-3,4,12,12a-tetrahydro-6H-pyrazino[2,1-c][1,4]benzoxazepin-2(1H)-yl]prop-2-en-1-one;
1-[(12aR)-10-chloro-9-(2-chloro-6-hydroxyphenyl)-7,8-difluoro-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-(difluoromethoxy)-10-fluoro-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-ethynyl-7,10-difluoro-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-(difluoromethoxy)-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-(cyclopropyloxy)-10-fluoro-3,4,12,12a-tetrahydro-6H-pyrazino[2,1-c][1,4]benzoxazepin-2(1H)-yl]prop-2-en-1-one;
1-((12aR)-9-(2-Chloro-6-hydroxyphenyl)-8-(3-(dimethylamino)prop-1-yn-1-yl)-10-fluoro-3,4,12,12a-tetrahydro-6H-benzo[f]pyrazino[2,1-c][1,4]oxazepin-2(1H)-yl)prop-2-en-1-one;
1-[(12aR)-9-(2-chloro-6-hydroxyphenyl)-10-fluoro-8-[(pyridin-4-yl)methoxy]-3,4,12,12a-tetrahydro-6H-pyrazino[2,1-c][1,4]benzoxazepin-2(1H)-yl]prop-2-en-1-one;
1-[(12aR)-10-chloro-9-(2-chloro-6-hydroxyphenyl)-8-(2-methoxyethoxy)-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-10-fluoro-8-[2-(piperidin-1-yl)ethoxy]-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-(prop-1-yn-1-yl)-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-2-[(<2>H3)methyloxy]-6a,7,9,10-tetrahydro-12H-pyrazino[2,1-c]pyrido[2,3-f][1,4]oxazepin-8(6H)-yl]prop-2-en-1-one;
1-[(12aR)-10-Chloro-9-(2-chloro-6-hydroxyphenyl)-8-(methoxymethyl)-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-7-[2-(dimethylamino)ethoxy]-10-fluoro-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-1-(prop-1-yn-1-yl)-6a,7,9,10-tetrahydro-12H-pyrazino[2,1-c]pyrido[3,4-f][1,4]oxazepin-8(6H)-yl]prop-2-en-1-one; and
1-((6aR)-4-Chloro-3-(2-chloro-6-hydroxyphenyl)-1-ethynyl-6a,7,9,10-tetrahydro-12H-pyrazino[2,1-c]pyrido[3,4-f][1,4]oxazepin-8(6H)-yl)prop-2-en-1-one; or a pharmaceutically acceptable salt thereof.

In another combination embodiment, the RASG12C inhibitor is a compound of formula (I), which is 1-[(12aR)-9-(2-chloro-6-hydroxyphenyl)-8-ethynyl-10-fluoro-3,4,12,12a-tetrahydro-6H-pyrazino[2,1-c][1,4]benzoxazepin-2(1H)-yl]prop-2-en-1-one.

In another combination embodiment, the RASG12C inhibitor is a compound of formula (I), which is 1-[(12aR)-9-(2-chloro-6-hydroxyphenyl)-10-fluoro-8-(prop-1-yn-1-yl)-3,4,12,12a-tetrahydro-6H-pyrazino[2,1-c][1,4]benzoxazepin-2(1H)-yl]prop-2-en-1-one.

In another embodiment of each of the combination embodiments described above, the antibody-drug conjugate as defined for the above combination embodiments is combined with a RASG12C inhibitor which is a compound represented by the following formula (II):

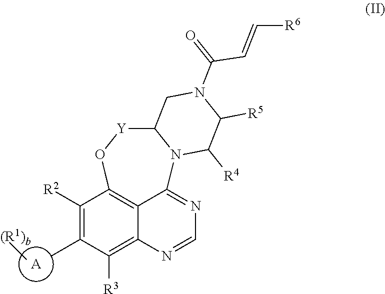

wherein:
Ring A is selected from phenyl and bicyclic heteroaryl;
R¹ in each occurrence is independently selected from C₁₋₄alkyl, halo, hydroxy, C₁₋₄alkoxy, C₁₋₃fluoroalkyl, C₁₋₃fluoroalkoxy, cyano and acetylenyl;
b is 0, 1, 2 or 3;
Y is CH₂ or CH₂CH₂;
R² is cyano, halo, C₁₋₄alkyl, C₁₋₄alkoxy or C₁₋₃fluoroalkyl;
R³ is F, Me, Et, MeO or C₁₋₂fluoroalkyl;
R⁴ is H or Me;
R⁵ is H or Me;
R⁶ is H or CH₂NMe₂;
or a pharmaceutically acceptable salt thereof, provided that when Y is CH₂, R² is Cl, R³ is F, A is phenyl,
b is 2, the groups R¹ are F and OH and are each ortho to the biaryl bond, and when both R⁴ and R⁶ are H, then R⁵ is Me.

In another combination embodiment, the antibody-drug conjugate as defined for the above combination embodiments is combined with a RASG12C inhibitor as defined above, having the structure:

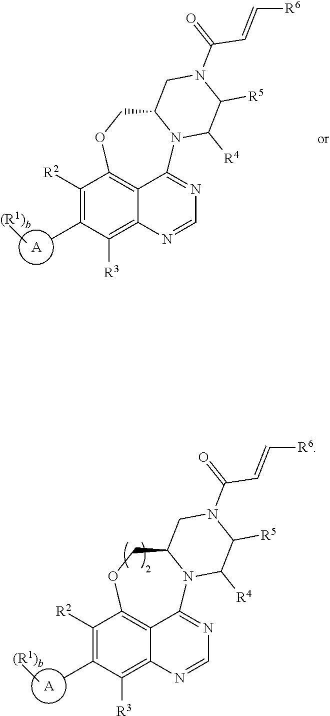

In another combination embodiment, the antibody-drug conjugate as defined for the above combination embodiments is combined with a RASG12C inhibitor as defined above wherein, in formula (II), the Ring A is selected from the group consisting

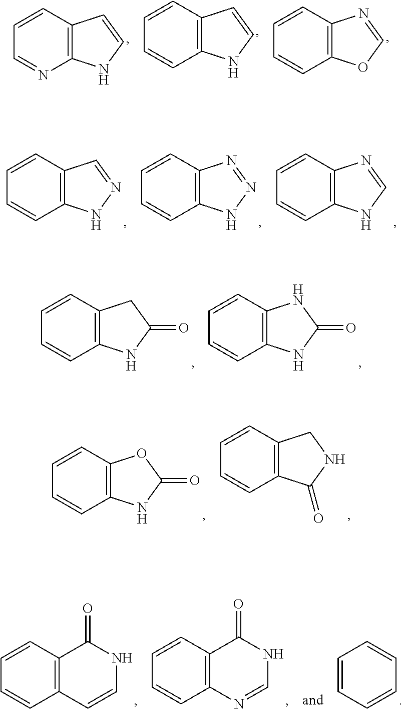

In another combination embodiment, the antibody-drug conjugate as defined for the above combination embodiments is combined with a RASG12C inhibitor as defined above wherein, in formula (II), R⁴ is H. In another embodiment, in formula (II), R⁶ is H. In another embodiment, in formula (II), Y is CH₂. In another embodiment, in formula (II), Y is CH₂CH₂. In another embodiment, in formula (II), R² is Cl. In another embodiment, in formula (II), R³ is F. In another embodiment, in formula (II), R⁴ is H and R⁵ is Me.

In another combination embodiment, the anti-HER2 antibody-drug conjugate as defined for the above combination embodiments is combined with a RASG12C inhibitor wherein the RASG12C inhibitor is a compound of formula (II) selected from:
7-[(8aS)-10-Acryloyl-6-chloro-4-fluoro-8,8a,9,10,11,12-hexahydropyrazino[2′,1′:3,4][1,4]oxazepino[5,6,7-de]quinazolin-5-yl]-6-methyl-2,3-dihydro-1H-isoindol-1-one;
1-[(8aS,11S)-6-Chloro-4-fluoro-5-(2-fluoro-6-hydroxyphenyl)-11-methyl-8a,9,11,12-tetrahydropyrazino[2′,1′:3,4][1,4]oxazepino[5,6,7-de]quinazolin-10(8H)-yl]prop-2-en-1-one;
1-[(8aS,11R)-6-Chloro-4-fluoro-5-(2-fluoro-6-hydroxyphenyl)-11-methyl-8a,9,11,12-tetrahydropyrazino[2′,1′:3,4][1,4]oxazepino[5,6,7-de]quinazolin-10(8H)-yl]prop-2-en-1-one;
5-[(8aS)-10-Acryloyl-6-chloro-4-fluoro-8,8a,9,10,11,12-hexa hydropyrazino[2′,1′:3,4][1,4]oxazepino[5,6,7-de]quinazolin-5-yl]-6-methylquinazolin-4(3H)-one;
1-[(8aS)-6-Chloro-4-fluoro-5-(5-methyl-1H-benzimidazol-4-yl)-8a,9,11,12-tetrahydropyrazino[2′,1′:3,4][1,4]oxazepino[5,6,7-de]quinazolin-10(8H)-yl]prop-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]isoquinolin-1(2H)-one;
1-[(8aS)-6-Chloro-4-fluoro-5-(1H-indazol-3-yl)-8a,9,11,12-tetrahydropyrazino[2′,1′:3,4][1,4]oxazepino[5,6,7-de]quinazolin-10(8H)-yl]prop-2-en-1-one;
1-[(8aS)-6-Chloro-4-fluoro-5-(2-hydroxy-6-methylphenyl)-8a,9,11,12-tetrahydropyrazino[2′,1′:3,4][1,4]oxazepino[5,6,7-de]quinazolin-10(8H)-yl]prop-2-en-1-one;
(2E)-1-[(8aS)-6-Chloro-4-fluoro-5-(2-fluoro-6-hydroxyphenyl)-8a,9,11,12-tetrahydropyrazino[2′,1′:3,4][1,4]oxazepino[5,6,7-de]quinazolin-10(8H)-yl]-4-(dimethylamino)but-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]-7-methylisoquinolin-1(2H)-one;
1-[(8aS)-6-Chloro-4-fluoro-5-(5-methyl-1H-benzotriazol-4-yl)-8a,9,11,12-tetrahydropyrazino[2′,1′:3,4][1,4]oxazepino[5,6,7-de]quinazolin-10(8H)-yl]prop-2-en-1-one;
1-((8aS)-6-chloro-4-fluoro-5-(5-fluoro-1H-benzo[d]imidazol-4-yl)-8a,9,11,12-tetrahydropyrazino[2′,1′:3,4][1,4]oxazepino[5,6,7-de]quinazolin-10(8H)-yl)prop-2-en-1-one;
1-[(8aS)-6-Chloro-4-fluoro-5-(5-fluoro-1H-indazol-4-yl)-8a,9,11,12-tetrahydropyrazino[2′,1′:3,4][1,4]oxazepino[5,6,7-de]quinazolin-10(8H)-yl]prop-2-en-1-one;
1-((8aS)-6-Chloro-4-fluoro-5-(5-fluoro-1-methyl-1H-benzo[d]imidazol-4-yl)-8a,9,11,12-tetrahydropyrazino[2′,1′:3,4][1,4]oxazepino[5,6,7-de]quinazolin-10(8H)-yl)prop-2-en-1-one;
1-[(8aS)-6-Chloro-4-fluoro-5-(5-fluoro-1H-benzotriazol-4-yl)-8a,9,11,12-tetrahydropyrazino[2′,1′:3,4][1,4]oxazepino[5,6,7-de]quinazolin-10(8H)-yl]prop-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]-7-fluoroisoquinolin-1(2H)-one;
(2E)-1-[(8aS)-6-chloro-4-fluoro-5-(5-methyl-1H-indazol-4-yl)-8a,9,11,12-tetrahydropyrazino[2′,1′:3,4][1,4]oxazepino[5,6,7-de]quinazolin-10(8H)-yl]-4-(dimethylamino)but-2-en-1-one;
1-[(6aR,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aR,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
8-[3-Chloro-1-fluoro-8-(prop-2-enoyl)-6,6a,7,8,9,10-hexahydro-5H-pyrazino[1′,2′:5,6][1,5]oxazocino[4,3,2-de]quinazolin-2-yl]-7-methylisoquinolin-1(2H)-one;
1-[(6aS,9R)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aR,9R)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aS,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(8aS)-4-Chloro-6-fluoro-5-(2-fluoro-6-hydroxyphenyl)-8a,9,11,12-tetrahydropyrazino[2′,1′:3,4][1,4]oxazepino[5,6,7-de]quinazolin-10(8H)-yl]prop-2-en-1-one;
1-[(8aS,11R)-6-Chloro-4-fluoro-11-methyl-5-(5-methyl-1H-benzimidazol-4-yl)-8a,9,11,12-tetrahydropyrazino[2′,1′:3,4][1,4]oxazepino[5,6,7-de]quinazolin-10(8H)-yl]prop-2-en-1-one;
8-[(8aS,11R)-6-Chloro-4-fluoro-11-methyl-10-(prop-2-enoyl)-8,8a,9,10,11,12-hexahydropyrazino[2′,1′:3,4][1,4]oxazepino[5,6,7-de]quinazolin-5-yl]-7-methylisoquinolin-1(2H)-one;
(2E)-1-[(8aS,11R)-6-Chloro-4-fluoro-11-methyl-5-(5-methyl-1H-benzimidazol-4-yl)-8a,9,11,12-tetrahydropyrazino[2′,1′:3,4][1,4]oxazepino[5,6,7-de]quinazolin-10(8H)-yl]-4-(dimethylamino)but-2-en-1-one; and
(2E)-1-[(8aS,11R)-6-Chloro-4-fluoro-11-methyl-5-(5-methyl-1H-indazol-4-yl)-8a,9,11,12-tetrahydropyrazino[2′,1′:3,4][1,4]oxazepino[5,6,7-de]quinazolin-10(8H)-yl]-4-(dimethylamino)but-2-en-1-one; or a pharmaceutically acceptable salt thereof.

In another combination embodiment, the anti-HER2 antibody-drug conjugate as defined for the above combination embodiments is combined with a RASG12C inhibitor wherein the RASG12C inhibitor is Compound A represented by the following formula:

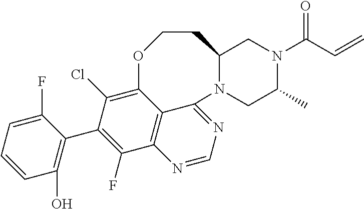 [SMILES: C=CC(=O)N1C[C@@H]2CCOc3c(Cl)c(-c4c(O)cccc4F)c(F)c4ncnc(c34)N2C[C@H]1C]

or a pharmaceutically acceptable salt thereof.

In another combination embodiment, the anti-HER2 antibody-drug conjugate as defined for the above combination embodiments is combined with a RASG12C inhibitor wherein the RASG12C inhibitor is selected from LY3537982 (Loxo/Lilly), AZD4625 (AstraZeneca), AMG510 (sotorasib: Amgen), MRTX849 (adagrasib: Mirati), JDQ443 (Novartis), GDC-6036 (Genentech), BI 1,823,911 (Boehringer Ingelheim), D1553 (InventisBio) and JNJ-74699157 (Johnson and Johnson).

In another combination embodiment, the anti-HER2 antibody-drug conjugate as defined for the above combination embodiments is combined with a RASG12C inhibitor wherein the RASG12C inhibitor is AMG510 (sotorasib).

In another combination embodiment, the anti-HER2 antibody-drug conjugate as defined above is combined with a RASG12C inhibitor wherein the RASG12C inhibitor is MRTX849 (adagrasib).

In a particularly preferred combination embodiment of the disclosure, the anti-HER2 antibody-drug conjugate is trastuzumab deruxtecan (DS-8201) and the RASG12C inhibitor is the compound represented by the following formula:

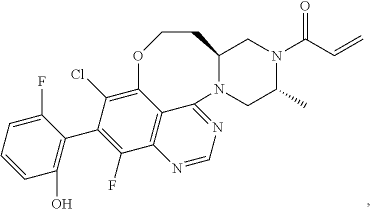 [SMILES: C=CC(=O)N1C[C@@H]2CCOc3c(Cl)c(-c4c(O)cccc4F)c(F)c4ncnc(c34)N2C[C@H]1C]

also identified as Compound A.

In another particularly preferred combination embodiment of the disclosure, the anti-HER2 antibody-drug conjugate is trastuzumab deruxtecan (DS-8201) and the RASG12C inhibitor is AMG510 (sotorasib).

In another particularly preferred combination embodiment of the disclosure, the anti-HER2 antibody-drug conjugate is trastuzumab deruxtecan (DS-8201) and the RASG12C inhibitor is MRTX849 (adagrasib).

6. Therapeutic Combined Use and Method Described in the following are a pharmaceutical product and a therapeutic use and method wherein the anti-HER2 antibody-drug conjugate according to the present disclosure and a RASG12C inhibitor are administered in combination.

The pharmaceutical product and therapeutic use and method of the present disclosure may be characterized in that the anti-HER2 antibody-drug conjugate and the RASG12C inhibitor are separately contained as active components in different formulations, and are administered simultaneously or at different times, or characterized in that the antibody-drug conjugate and the RASG12C inhibitor are contained as active components in a single formulation and administered.

In the pharmaceutical product and therapeutic method of the present disclosure, a single RASG12C inhibitor used in the present disclosure can be administered in combination with the anti-HER2 antibody-drug conjugate, or two or more different RASG12C inhibitors can be administered in combination with the antibody-drug conjugate.

The pharmaceutical product and therapeutic method of the present disclosure can be used for treating cancer, and can be preferably used for treating at least one cancer selected from the group consisting of breast cancer (including triple negative breast cancer and luminal breast cancer), gastric cancer (also called gastric adenocarcinoma), colorectal cancer (also called colon and rectal cancer, and including colon cancer and rectal cancer), lung cancer (including small cell lung cancer and non-small cell lung cancer), esophageal cancer, head-and-neck cancer (including salivary gland cancer and pharyngeal cancer), esophagogastric junction adenocarcinoma, biliary tract cancer (including bile duct cancer), Paget's disease, pancreatic cancer, ovarian cancer, uterine carcinosarcoma, urothelial cancer, prostate cancer, bladder cancer, gastrointestinal stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, and melanoma, and can be more preferably used for treating at least one cancer selected from the group consisting of breast cancer, gastric cancer, colorectal cancer, lung cancer (preferably non-small cell lung cancer), pancreatic cancer, ovarian cancer, prostate cancer, and kidney cancer.

The presence or absence of HER2 tumor markers can be determined, for example, by collecting tumor tissue from a cancer patient to prepare a formalin-fixed, paraffin-embedded (FFPE) specimen and subjecting the specimen to a test for gene products (proteins), for example, with an immunohistochemical (IHC) method, a flow cytometer, or Western blotting, or to a test for gene transcription, for example, with an in situ hybridization (ISH) method, a quantitative PCR method (q-PCR), or microarray analysis, or by collecting cell-free circulating tumor DNA (ctDNA) from a cancer patient and subjecting the ctDNA to a test with a method such as next-generation sequencing (NGS).

The pharmaceutical product and therapeutic method of the present disclosure can be used for HER2-expressing cancer, which may be HER2-overexpressing cancer (high or moderate) or may be HER2 low-expressing cancer.

In the present disclosure, the term “HER2-overexpressing cancer” is not particularly limited as long as it is recognized as HER2-overexpressing cancer by those skilled in the art. Preferred examples of the HER2-overexpressing cancer can include cancer given a score of 3+ for the expression of HER2 in an IHC method, and cancer given a score of 2+ for the expression of HER2 in an IHC method and determined as positive for the expression of HER2 in an in situ hybridization method (ISH). The in situ hybridization method of the present disclosure includes a fluorescence in situ hybridization method (FISH) and a dual color in situ hybridization method (DISH).

In the present disclosure, the term “HER2 low-expressing cancer” is not particularly limited as long as it is recognized as HER2 low-expressing cancer by those skilled in the art. Preferred examples of the HER2 low-expressing cancer can include cancer given a score of 2+ for the expression of HER2 in an IHC method and determined as negative for the expression of HER2 in an in situ hybridization method, and cancer given a score of 1+ for the expression of HER2 in an IHC method.

The method for scoring the degree of HER2 expression by the IHC method, or the method for determining positivity or negativity to HER2 expression by the in situ hybridization method is not particularly limited as long as it is recognized by those skilled in the art. Examples of the method can include a method described in the 4th edition of the guidelines for HER2 testing, breast cancer (developed by the Japanese Pathology Board for Optimal Use of HER2 for Breast Cancer).

The cancer, particularly in regard to the treatment of breast cancer, may be HER2-overexpressing (high or moderate) or low-expressing breast cancer, or triple-negative breast cancer, and/or may have a HER2 status score of IHC 3+, IHC 2+, IHC 1+ or IHC>0 and <1+.

The pharmaceutical product and therapeutic method of the present disclosure can be preferably used for a mammal, but are more preferably used for a human.

The antitumor effect of the pharmaceutical product and therapeutic method of the present disclosure can be confirmed by transplanting cancer cells to a test subject animal to prepare a model and measuring reduction in tumor volume or life-prolonging effect by application of the pharmaceutical product and therapeutic method of the present disclosure. And then, the effect of combined use of the antibody-drug conjugate used in the present disclosure and a RASG12C inhibitor can be confirmed by comparing antitumor effect with single administration of the antibody-drug conjugate used in the present disclosure and that of the RASG12C inhibitor.

The antitumor effect of the pharmaceutical product and therapeutic method of the present disclosure can be confirmed in a clinical trial using any of an evaluation method with Response Evaluation Criteria in Solid Tumors (RECIST), a WHO evaluation method, a Macdonald evaluation method, body weight measurement, and other approaches, and can be determined on the basis of indexes of complete response (CR), partial response (PR); progressive disease (PD), objective response rate (ORR), duration of response (DoR), progression-free survival (PFS), overall survival (OS), and so on.

By using the above methods, the superiority in antitumor effect of the pharmaceutical product and therapeutic method of the present disclosure to existing pharmaceutical products and therapeutic methods for cancer treatment can be confirmed.

The pharmaceutical product and therapeutic method of the present disclosure can delay development of cancer cells, inhibit growth thereof, and further kill cancer cells. These effects can allow cancer patients to be free from symptoms caused by cancer or achieve improvement in quality of life (QOL) of cancer patients and attain a therapeutic effect by sustaining the lives of the cancer patients. Even if the pharmaceutical product and therapeutic method of the present disclosure do not accomplish killing cancer cells, they can achieve higher QOL of cancer patients while achieving longer-term survival, by inhibiting or controlling the growth of cancer cells.

The pharmaceutical product of the present disclosure can be expected to exert a therapeutic effect by application as systemic therapy to patients, and additionally, by local application to cancer tissues.

The pharmaceutical product and therapeutic method of the present disclosure, in another aspect, provides for use as an adjuct in cancer therapy with ionizing radiation or other chemotherapeutic agents. For example, in the treatment of cancer, the treatment may comprise administering to a subject in need of treatment an effective amount of the pharmaceutical product, simultaneously or sequentially with ionizing radiation or other chemotherapeutic agents.

The pharmaceutical product and therapeutic method of the present disclosure can be used as adjuvant chemotherapy combined with surgery operation. The pharmaceutical product of the present disclosure may be administered for the purpose of reducing tumor size before surgical operation (referred to as preoperative adjuvant chemotherapy or neoadjuvant therapy), or may be administered for the purpose of preventing recurrence of tumor after surgical operation (referred to as postoperative adjuvant chemotherapy or adjuvant therapy).

In some embodiments, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies, et al., Cell, 115, 523-535). BRCA1 and BRCA2 are known tumour suppressors whose wild-type alleles are frequently lost in tumours of heterozygous carriers (Jasin M., Oncogene, 21(58), 8981-93 (2002); Tutt, et al., Trends Mol Med., 8 (12), 571-6, (2002)). The association of BRCA1 and/or BRCA2 mutations with breast cancer is well-characterised in the art (Radice, P. J., Exp Clin Cancer Res., 21(3 Suppl), 9-12 (2002)). Amplification of the EMSY gene, which encodes a BRCA2 binding factor, is also known to be associated with breast and ovarian cancer. Carriers of mutations in BRCA1 and/or BRCA2 are also at elevated risk of certain cancers, including breast, ovary, pancreas, prostate, hematological, gastrointestinal and lung cancer. In some embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen, S. L. and Ostrander, E. A., Genet. Test, 1, 75-83 (1992); Chappnis, P. O. and Foulkes, W. O., Cancer Treat Res, 107, 29-59 (2002); Janatova M., et al., Neoplasma, 50(4), 246-505 (2003); Jancarkova, N., Ceska Gynekol., 68{1), 11-6 (2003)). Determination of amplification of the BRCA2 binding factor EMSY is described in Hughes-Davies, et al., Cell, 115, 523-535).

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

The pharmaceutical product of the present disclosure can be administered containing at least one pharmaceutically suitable ingredient. Pharmaceutically suitable ingredients can be suitably selected and applied from formulation additives or the like that are generally used in the art, in accordance with the dosage, administration concentration, or the like of the antibody-drug conjugate used in the present disclosure and a RASG12C inhibitor. The anti-HER2 antibody-drug conjugate used in the present disclosure can be administered, for example, as a pharmaceutical product containing a buffer such as histidine buffer, a vehicle such as sucrose and trehalose, and a surfactant such as Polysorbates 80 and 20. The pharmaceutical product containing the antibody-drug conjugate used in the present disclosure can be preferably used as an injection, can be more preferably used as an aqueous injection or a lyophilized injection, and can be even more preferably used as a lyophilized injection. In the case that the pharmaceutical product containing the anti-HER2 antibody-drug conjugate used in the present disclosure is an aqueous injection, the aqueous injection can be preferably diluted with a suitable diluent and then given as an intravenous infusion. Examples of the diluent can include dextrose solution and physiological saline, dextrose solution can be preferably exemplified, and 5% dextrose solution can be more preferably exemplified. In the case that the pharmaceutical product of the present disclosure is a lyophilized injection, a required amount of the lyophilized injection dissolved in advance in water for injection can be preferably diluted with a suitable diluent and then given as an intravenous infusion. Examples of the diluent can include dextrose solution and physiological saline, dextrose solution can be preferably exemplified, and 5% dextrose solution can be more preferably exemplified.

Examples of the administration route applicable to administration of the pharmaceutical product of the present disclosure can include intravenous, intradermal, subcutaneous, intramuscular, and intraperitoneal routes, and intravenous routes are preferred.

The anti-HER2 antibody-drug conjugate used in the present disclosure can be administered to a human with intervals of 1 to 180 days, can be preferably administered with intervals of a week, two weeks, three weeks, or four weeks, and can be more preferably administered with intervals of three weeks. The anti-HER2 antibody-drug conjugate used in the present disclosure can be administered in a dose of about 0.001 to 100 mg/kg per administration, and can be preferably administered in a dose of 0.8 to 12.4 mg/kg per administration. For example, the anti-HER2 antibody-drug conjugate can be administered once every three weeks at a dose of 0.8 mg/kg, 1.6 mg/kg, 3.2 mg/kg, 5.4 mg/kg, 6.4 mg/kg, 7.4 mg/kg, or 8 mg/kg, and can be preferably administered once every three weeks at a dose of 5.4 mg/kg or 6.4 mg/kg.

The RASG12C selective inhibitor may be administered in a suitable dose by any suitable route of administration. The size of the dose required for the therapeutic treatment of a particular disease state will necessarily be varied depending on the subject treated, the route of administration and the severity of the illness being treated. For further information on routes of administration and dosage regimes, reference may be made to Chapter 25.3 in Volume 5 of Comprehensive Medicinal Chemistry (Corwin Hansch; Chairman of Editorial Board), Pergamon Press 1990.

The compound of Formula (I) or (II), or a pharmaceutically acceptable salt thereof, will normally be administered via the oral route though parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable salt or solvate thereof, or a solvate of such a salt, in a pharmaceutically acceptable dosage form may be possible. Depending upon the disorder and patient to be treated and the route of administration, the compositions may be administered at varying doses, for example in an oral dose of from 1 mg to 1,000 mg or from 100 mg to 2,000 mg. The pharmaceutical formulations of the compound of Formula (I) or (II) described above may be prepared e.g. for parenteral, subcutaneous, intramuscular or intravenous administration. The pharmaceutical formulations of the compound of Formula (I) or (II) described above may conveniently be administered in unit dosage form and may be prepared by any of the methods well-known in the pharmaceutical art, for example as described in Remington's Pharmaceutical Sciences, 17th ed., Mack Publishing Company, Easton, PA., (1985). Pharmaceutical formulations suitable for oral administration may comprise one or more physiologically compatible carriers and/or excipients and may be in solid or liquid form. Tablets and capsules may be prepared with binding agents; fillers; lubricants; and surfactants. Liquid compositions may contain conventional additives such as suspending agents; emulsifying agents; and preservatives Liquid compositions may be encapsulated in, for example, gelatin to provide a unit dosage form. Solid oral dosage forms include tablets, two-piece hard shell capsules and soft elastic gelatin (SEG) capsules. An exemplary oral composition would comprise a compound of Formula (I) or (II) and at least one pharmaceutically acceptable excipient filled into a two-piece hard shell capsule or a soft elastic gelatin (SEG) capsule.

### EXAMPLES

The present disclosure is specifically described in view of the examples shown below. However, the present disclosure is not limited to these. Further, it is by no means to be interpreted in a limited way.

### Example 1: Production of Antibody-Drug Conjugate

In accordance with a production method described in WO2015/115091 and using an anti-HER2 antibody (an antibody comprising a heavy chain consisting of an amino acid sequence represented by SEQ ID NO: 11 (amino acid residues 1 to 449 of SEQ ID NO: 1) and a light chain consisting of an amino acid sequence consisting of all amino acid residues 1 to 214 of SEQ ID NO: 2), an anti-HER2 antibody-drug conjugate in which a drug-linker represented by the following formula:

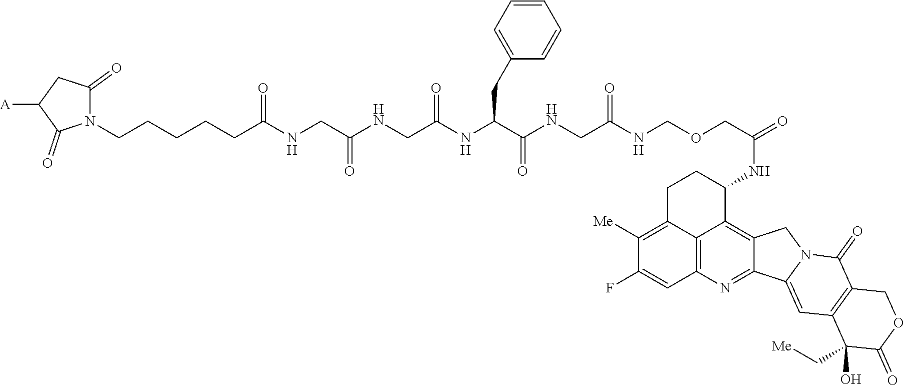

wherein A represents the connecting position to an antibody, is conjugated to the anti-HER2 antibody via a thioether bond was produced (DS-8201: trastuzumab deruxtecan). The DAR of the antibody-drug conjugate is 7.7 or 7.8.

### Example 2: Production of RASG12C Inhibitor

In accordance with a known production method, a RASG12C inhibitor of formula (I) is prepared. Specifically, 1-[(6aS,9R)-3-chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one:

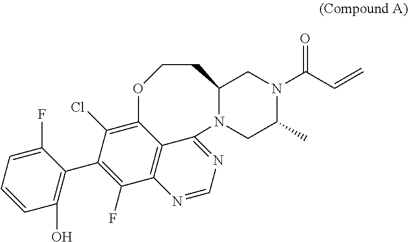 [SMILES: C=CC(=O)N1C[C@@H]2CCOc3c(Cl)c(-c4c(O)cccc4F)c(F)c4ncnc(c34)N2C[C@H]1C]

can be prepared according to Example 39 in WO2019/215203.

### Example 3: Antitumor Tests

Combination of antibody-drug conjugate DS-8201 (trastuzumab deruxtecan (Enhertu®)) with KRAS G12C inhibitors.

To assess the benefit of combining KRAS G12C inhibitors with DS-8201, in vitro cellular proliferation assays in a range of KRAS^{G12C} mutant human cell lines were conducted. 5 day cellular proliferation assays using dose ranges of inhibitors were used to explore the synergistic combination potential of KRAS G12C inhibitors and DS-8201. Extended duration (20 day) cellular proliferation assays, that included monitoring of growth following drug treatment cessation were utilised to explore depth & duration of combination benefit between Compound A as KRAS G12C inhibitor and DS-8201.

Anti-tumour combination benefit of KRAS G12C inhibitors with DS-8201 was explored in vivo in KRAS G12C mutant lung xenograft and colorectal patient derived explant models.

### Method

### Preclinical Models

NCI-H358 (CRL-5807), NCI-H2122 (CRL-5985), NCI-H1792 (CRL-5895), SW1573 (CRL-2170) and SW837 (CCL-235) cells were obtained from the American Type Culture Collection (ATCC). LIM2099 (12062002) & KYSE410 (94072023) cells were from the European Collection of Authenticated Cell Cultures (ECACC). LU99 cells (JCRB0080) were obtained from the Japanese Collection of Research Bioresources (JCRB) and HCC44 (70044) were from the Korean Cell line bank (KCLB). Cell lines were authenticated by short-tandem repeat analysis (STR). CTG-1489 and CTG-0387 are colorectal cancer patient derived explant (PDX) models from Champions Oncology.

### In Vitro Studies

Cell lines were routinely cultured in Growth Media (RPMI-1640 without phenol red, 10% FCS, 2 mM L-glutamine). For seeding cells into plates for assays, the cells were detached with 0.25% Trypsin solution, and then syringed 3 times through an 18G blunt ended needle to ensure cells were fully dispersed.

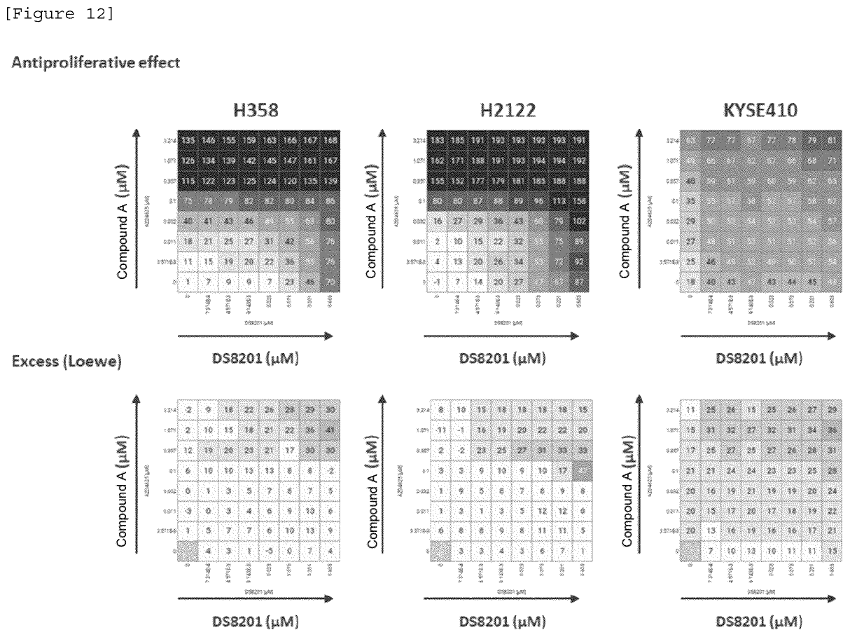

For determination of synergistic combination effects, cells were seeded into 384-well plates in 70 μl of growth media at 500-1000 cells/well. Plates were incubated for 24 hrs and either processed immediately (day 0) or treated with KRAS G12C inhibitor (Compound A) and DS-8201, using a 8×8 dosing matrix, with an ECHO 555 liquid handler (Labcyte) and incubated for a further 5 days. Cell growth was assessed by adenosine triphosphate content using CellTiter-Glo (Promega). 35 μl of CellTiter-Glo reagent was added and incubated for 1 hour at room temperature. The luminescence was measured in SpectraMax i3 (Molecular Devices) plate reader. Data was normalised to day 0 and Two-dimensional dose response matrix and curve fitting were processed in the combination extension of Genedata Screener12™ (Genedata, Basel, Switzerland). Combination activity (synergism) was calculated using the Loewe dose-additivity model. This model of additivity provides a null-reference that is predicted by the expected response if the two agents were the same drug. The 3-dimensional model surface, predicted from the two single-agent response curves, is subtracted from the experimentally-derived 3-dimensional dose effect surface to generate a difference volume. This excess matrix volume can be integrated to generate a synergy score. Synergistic combinations are defined to have a Loewe score of ≥5. The results as combination matrices are shown in FIG. 12.

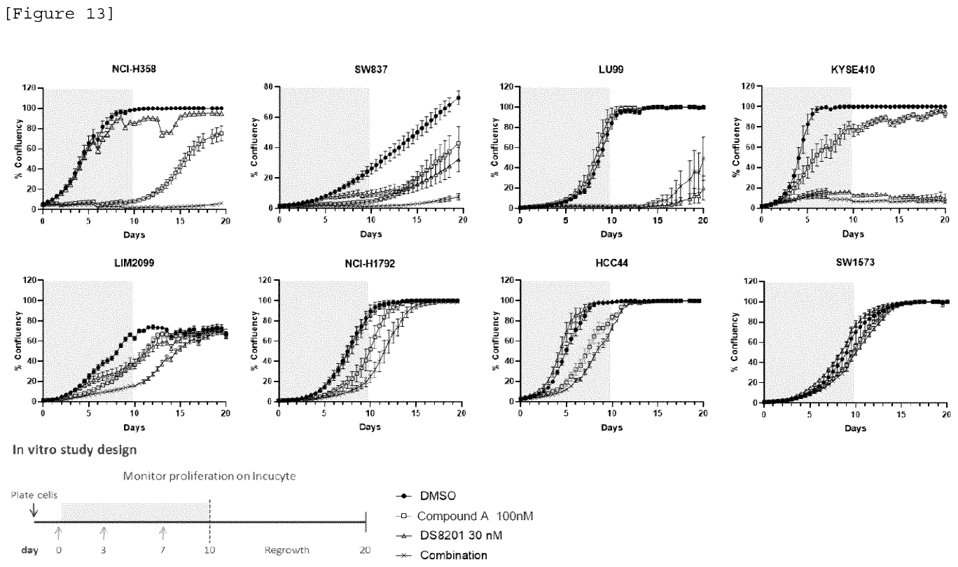

For time-to-regrowth assays cells were seeded into 48 well tissue culture plate in growth media and left to attach overnight. After 24 hours cells were dosed with KRAS G12C inhibitor (Compound A) and/or DS-8201. Cells were re-dosed with fresh media containing inhibitors on the 4th and 7th day, and on 10th day fresh-media was added with no inhibitors. The confluency of the cells was monitored using the Incucyte Zoom live cell imaging platform (Sartorius). The results are shown in the graphs in FIG. 13.

Loewe synergy scores were obtained for combined treatment of KRAS G12C inhibitors (Compound A; AMG510; MRTX849) and DS-8201 in a panel of KRAS G12C mutant cancer cell lines. Synergy scores were derived from independent combination experiments. Synergistic combinations are defined to have a Loewe score of ≥5. The results are shown in Table 1 below:

TABLE 1

| | | Compound A + | AMG510 + | MRTX849 + |
|---|---|---|---|---|
| | | DS-8201 | DS-8201 | DS-8201 |
| | | synergy | synergy | synergy |
| | Disease | score | score | score |
| Cell line | type | Loewe model | Loewe model | Loewe model |
| NCI-H358 | NSCLC | 7.5, 8.2, | 10.8 | 6.2 |
| | | 10.3, 4.9, | | |
| | | 7.8, 7.0 | | |
| NCI-H2122 | NSCLC | 12.5, 12.9, | 27.1 | 17.2 |
| | | 11.8, 6.9, | | |
| | | 13.7, 15.3 | | |
| HCC44 | NSCLC | 2.0, 1.4, | — | — |
| | | 0.5, 0.9 | | |
| NCI-H1792 | NSCLC | 1.7, 1.4, | — | — |
| | | 1.5, 1.3 | | |
| SW1573 | NSCLC | 1.6, 3.6, | — | — |
| | | 3.7, 3.9 | | |
| LU99 | NSCLC | 3.6 | — | — |
| SW837 | CRC | 2.1, 1.8, | — | — |
| | | 2.0, 2.5 | | |
| LIM2099 | CRC | −0.4, −0.1, | — | — |
| | | −0.1, 0.4 | | |
| KYSE410 | Oesophageal | 9.1, 10.6, | — | — |
| | | 8.9, 1.9, | | |
| | | 7.9 | | |

The results in FIG. 12 show that Compound A exhibits synergistic combination activity with DS-8201 in vitro, in KRAS^{G12C} lung cancer cell lines H358 and H2122, and in KRAS^{G12C} oesophageal cancer cell line KYSE410.

The results in FIG. 13 show that Compound A and DS-8201 combinations have increased tumour growth inhibition and delay regrowth on treatment cessation in vitro, in lung, colorectal and oesophageal cancer cell lines.

The results in Table 1 show that AMG510 and MRTX849 exhibit synergistic combination activity with DS-8201 in vitro, in KRAS^{G12C} lung, colorectal and oesophageal cancer cell lines, and that DS-8201 and Compound A exhibit synergistic combination activity in KRAS^{G12C} lung cancer cell lines.

### In Vivo Studies

In vivo studies were performed at AstraZeneca (xenograft models) or Champions Oncology (PDX models) in accordance with local regulations in Athymic Nude-Foxn1nu mice (Envigo). For NCI-H358, NCI-H2122 or LU99 xenograft studies cells were prepared on the day of implant at a concentration of 3×10⁶, 2.5×10⁵ or 5×10⁶ cells respectively in a 50:50 ratio of Matrigel:cells. Cell suspensions were implanted at 0.1 ml/mouse subcutaneously into the left flank. Animals with actively growing tumours were randomized into the relevant treatment groups once a mean tumour volume of approximately 0.2-0.3 cm³ was reached. For the patient derived explant (PDX) studies, animals were implanted with the relevant PDX tumour fragment on the left flank and tumour volumes recorded seven to ten days after implantation. Once tumours reached an average tumour volume of 0.15-0.3 cm³ actively growing tumours were then randomized into the relevant treatment groups.

The day following randomization treatments were initiated. Animals were given orally a single daily dose of vehicle control or 100 mg/kg Compound A for the treatment period indicated. DS-8201 was given as a single intravenous dose. Compound A was formulated in 0.5% HPMC/0.1% Tween80. DS-8201 was formulated in 25 mM Histidine Buffer-9% Sucrose, pH 5.5. Tumours were measured twice weekly by caliper and volume calculated using elliptical formula (pi/6×width×width×length). Animal bodyweight and tumour condition were also recorded twice weekly for the duration of the study.

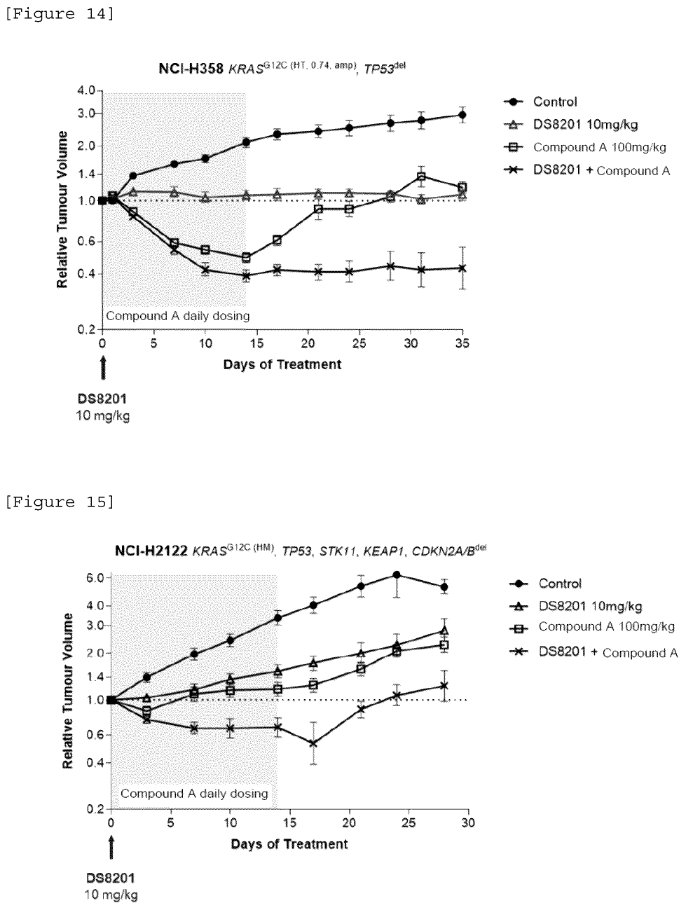

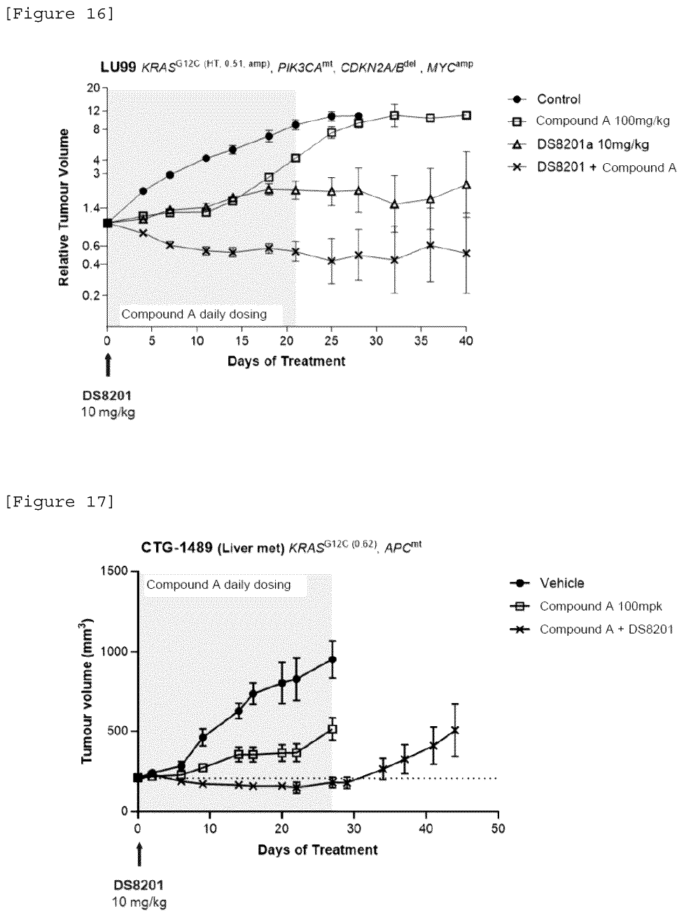

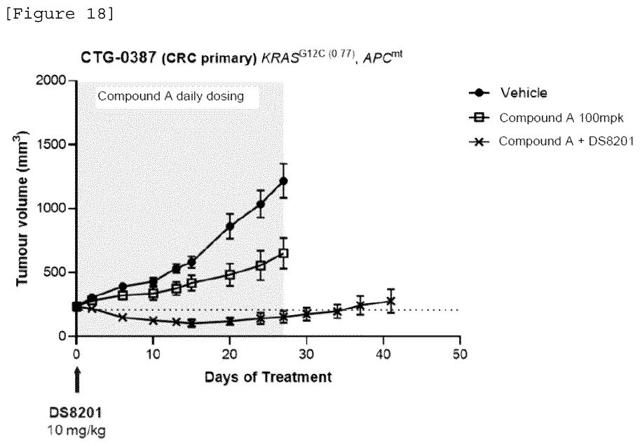

The results are shown in the graphs in in FIGS. 14 to 18. As seen from the results, Compound A and DS-8201 combinations exhibit enhanced and prolonged tumour growth inhibition in vivo, in KRAS^{G12C} mutant lung cancer xenograft models NCI-H358, NCI-H2122 and LU99, and in KRAS^{G12C} mutant colorectal cancer patient derived explant models CTG-1489 and CTG-0387.

Thus, the results in Example 3 (FIGS. 12-18; Table 1) demonstrate that KRAS G12C inhibition using a KRAS G12C inhibitor (Compound A) enhances the antitumor efficacy of an anti-HER2 antibody-drug conjugate (DS-8201) in HER2-expressing cell lines in vitro and in vivo.

### Example 4: Antitumor Tests

Combination of antibody-drug conjugate T-DM1 (trastuzumab emtansine (Kadcyla®)) with KRAS G12C inhibitors.

To assess the benefit of combining KRAS G12C inhibitors with T-DM1, in vitro cellular proliferation assays in a range of KRAS^{G12C} mutant human cell lines were conducted. 5 day cellular proliferation assays using dose ranges of inhibitors were used to explore the synergistic combination potential of KRAS G12C inhibitors and DS-8201.

### Method

### Preclinical Models

NCI-H358 (CRL-5807) and NCI-H2122 (CRL-5985) cells were obtained from the American Type Culture Collection (ATCC). Cell lines were authenticated by short-tandem repeat analysis (STR).

### In Vitro Studies

Cell lines were routinely cultured in Growth Media (RPMI-1640 without phenol red, 10% FCS, 2 mM L-glutamine). For seeding cells into plates for assays, the cells were detached with 0.25% Trypsin solution, and then syringed 3 times through an 18G blunt ended needle to ensure cells were fully dispersed.

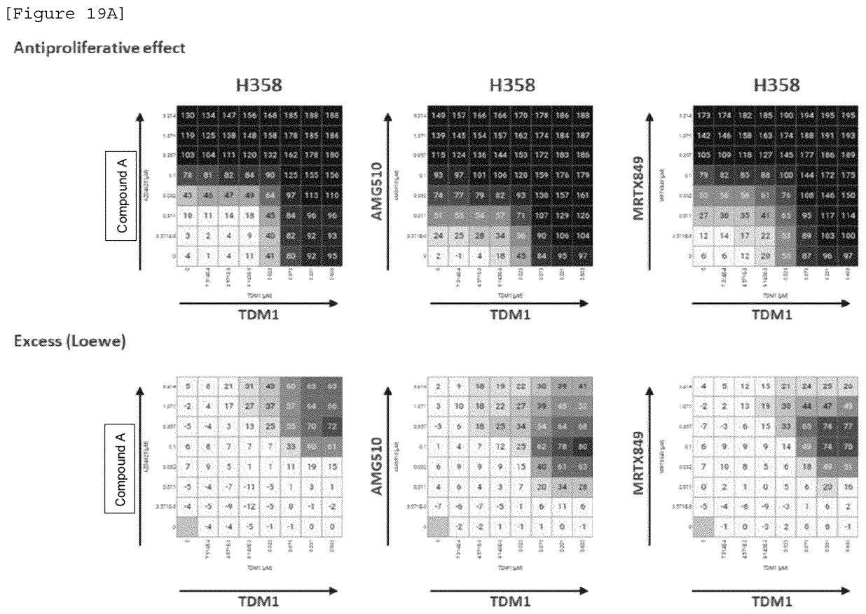

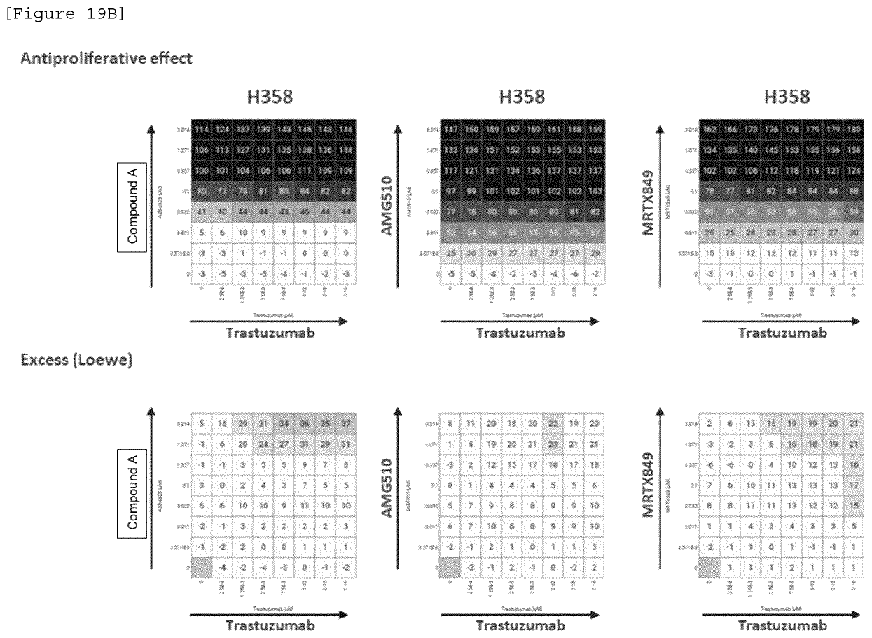

For determination of synergistic combination effects, cells were seeded into 384-well plates in 70 μl of growth media at 500-1000 cells/well. Plates were incubated for 24 hrs and either processed immediately (day 0) or treated with KRAS G12C inhibitor (Compound A) and T-DM1 or trastuzumab, using a 8×8 dosing matrix, with an ECHO 555 liquid handler (Labcyte) and incubated for a further 5 days. Cell growth was assessed by adenosine triphosphate content using CellTiter-Glo (Promega). 35 μl of CellTiter-Glo reagent was added and incubated for 1 hour at room temperature. The luminescence was measured in SpectraMax i3 (Molecular Devices) plate reader. Data was normalised to day 0 and Two-dimensional dose response matrix and curve fitting were processed in the combination extension of Genedata Screener12™ (Genedata, Basel, Switzerland). Combination activity (synergism) was calculated using the Loewe dose-additivity model. This model of additivity provides a null-reference that is predicted by the expected response if the two agents were the same drug. The 3-dimensional model surface, predicted from the two single-agent response curves, is subtracted from the experimentally-derived 3-dimensional dose effect surface to generate a difference volume. This excess matrix volume can be integrated to generate a synergy score. Synergistic combinations are defined to have a Loewe score of ≥5. The results as combination matrices are shown in FIGS. 19A and 19B.

Loewe synergy scores were obtained for combined treatment of KRAS G12C inhibitors (Compound A; AMG510; MRTX849) and T-DM1 or trastuzumab in NCI-H358 and NCI-H2122 cancer cell lines. Synergy scores were derived from independent combination experiments. Synergistic combinations are defined to have a Loewe score of ≥5. The results are shown in Table 2 below:

TABLE 2

| | | Compound | AMG510 + | MRTX849 + | Compound A + | AMG510 + | MRTX849 + |
|---|---|---|---|---|---|---|---|
| | | A + T-DM1 | T-DM1 | T-DM1 | Trastuzumab | Trastuzumab | Trastuzumab |
| | | Synergy | Synergy | Synergy | Synergy | Synergy | Synergy |
| | | score | score | score | score | score | score |
| Cell | Disease | Loewe | Loewe | Loewe | Loewe | Loewe | Loewe |
| line | type | model | model | model | model | model | model |
| NCI- | NSCLC | 20.43, 24.01 | 24.83 | 20.98 | 7.34, 5.21 | 7.84 | 6.02 |
| H358 | | | | | | | |
| NCI- | NSCLC | 7.78, 1.48 | 10.91 | 8.63 | 7.85, 7.21 | 17.41 | 7.0 |
| H2122 | | | | | | | |

The results in FIGS. 19A and 19B show that Compound A, AMG510 and MRTX849 exhibit synergistic combination activity with T-DM1, but not with trastuzumab, in vitro, in KRAS^{G12C} lung cancer cell line H358.

The results in Table 2 show that Compound A, AMG510 and MRTX849 exhibit synergistic combination activity with T-DM1 in vitro, in KRAS^{G12C} lung cancer cell lines NCI-H358 and NCI-H2122.

Thus, the results in Example 4 (FIG. 19A; Table 2) demonstrate that KRAS G12C inhibition using a KRAS G12C inhibitor (Compound A; AMG510; MRTX849) enhances the antitumor efficacy of an anti-HER2 antibody-drug conjugate (T-DM1) in HER2-expressing lung cancer cell lines in vitro.

The foregoing written specification is considered to be sufficient to enable one skilled in the art to practice the embodiments. The foregoing description and Examples detail certain embodiments and describe the best mode contemplated by the inventors. It will be appreciated, however, that no matter how detailed the foregoing may appear in text, the embodiments may be practiced in many ways and the claims include any equivalents thereof.

### Free Text of Sequence Listing

SEQ ID NO: 1—Amino acid sequence of a heavy chain of an anti-HER2 antibody
SEQ ID NO: 2—Amino acid sequence of a light chain of an anti-HER2 antibody
SEQ ID NO: 3—Amino acid sequence of a heavy chain CDRH1 [=amino acid residues 26 to 33 of SEQ ID NO: 1]
SEQ ID NO: 4—Amino acid sequence of a heavy chain CDRH2 [=amino acid residues 51 to 58 of SEQ ID NO: 1]
SEQ ID NO: 5—Amino acid sequence of a heavy chain CDRH3 [=amino acid residues 97 to 109 of SEQ ID NO: 1]
SEQ ID NO: 6—Amino acid sequence of a light chain CDRL1 [=amino acid residues 27 to 32 of SEQ ID NO: 2]
SEQ ID NO: 7—Amino acid sequence comprising amino acid sequence of a light chain CDRL2 (SAS) [=amino acid residues 50 to 56 of SEQ ID NO: 2]
SEQ ID NO: 8—Amino acid sequence of a light chain CDRL3 [=amino acid residues 89 to 97 of SEQ ID NO: 2]
SEQ ID NO: 9—Amino acid sequence of a heavy chain variable region [=amino acid residues 1 to 120 of SEQ ID NO: 1]
SEQ ID NO: 10—Amino acid sequence of a light chain variable region [=amino acid residues 1 to 107 of SEQ ID NO: 2]
SEQ ID NO: 11—Amino acid sequence of a heavy chain [=amino acid residues 1 to 449 of SEQ ID NO: 1]

1. A pharmaceutical product comprising an antibody-drug conjugate in which the antibody is an anti-HER2 antibody, and a RASG12C inhibitor for administration in combination.

2. The pharmaceutical product according to claim 1, wherein the RASG12C inhibitor is a compound represented by the following formula (I): 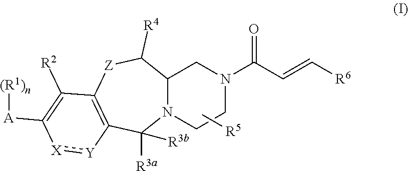
wherein:
A is phenyl or a bicyclic heteroaryl group;
X and Y are connected by a double bond and i) X is CR⁷ and Y is CR⁸, ii) X is N and Y is CR⁸, or iii) X is CR⁷ and Y is N; or
X and Y together are C(O)NR⁹; or
X and Y are adjacent ring atoms of an optionally substituted 5- or 6-membered N-heterocycle fused to the aromatic ring substituted with Z, and X and Y are both C or are C and N;
Z is O, NH, or NMe;
R¹ is independently selected from F, Cl, Br, OH, CH₂OH, OMe, CH₂OMe, C₁-C₃alkyl and C₁-C₃fluoroalkyl;
n is 0, 1, 2 or 3;
R² is H, F, Cl, CCH, CCMe, CN, Br, C₁-C₃alkyl, C₁-C₃fluoroalkyl, OMe or OEt;
R^{3a} and R^{3b} together are ═O or R^{3a} and R^{3b} are H;
R⁴ is H or Me;
R⁵ is H or Me;
R⁶ is H or CH₂NMe₂;
R⁷ and R⁸ are independently selected from H, F, Cl, CCH, CC(C₁-C₃alkyl), CCCH₂NMe₂, CCCH₂O(C₁-C₃alkyl), CN, Me, C₁-C₆alkyl, OH, OMe, O(C₁-C₃alkyl), O(C₁-C₃deuteroalkyl), O(C₁-C₃fluoroalkyl), O(C₃-C₆cycloalkyl), C₁-C₃fluoroalkyl, OCH₂CH₂NMe₂, OCH₂CH₂OMe, CH₂OMe, OCH₂CH₂N(CH₂CH₂)₂CH, OCH₂CH₂N(CH₂CH₂)₂O, OCH₂CH₂ (2-pyridyl) or an optionally substituted 3-, 4-, 5- or 6-membered carbocycle or heterocycle; or
R⁷ and R⁸ combine to form an optionally substituted 5- or 6-membered carbocycle or heterocycle;
R⁹ is selected from H, Me, Et, C₃H₇ and C₁-C₃fluoroalkyl;
or a pharmaceutically acceptable salt thereof.

3. The pharmaceutical product according to claim 2 wherein, in formula (I), i) X is CR⁷ and Y is CR⁸, ii) X is N and Y is CR⁸ or iii) X is CR⁷ and Y is N.

4. The pharmaceutical product according to claim 2 or claim 3 wherein, in formula (I), Z is O.

5. The pharmaceutical product according to any one of claims 2 to 4 wherein, in formula (I), R^{3a} and R^{3b} are H.

6. The pharmaceutical product according to any one of claims 2 to 5 wherein, in formula (I), R⁴ is H.

7. The pharmaceutical product according to any one of claims 2 to 6 wherein, in formula (I), R⁶ is H.

8. The pharmaceutical product according to any one of claims 2 to 7 wherein, in formula (I), A is phenyl.

9. The pharmaceutical product according to claim 2, wherein the RASG12C inhibitor is a compound selected from:
(12aS)-2-Acryloyl-10-chloro-9-(5-methyl-1H-indazol-4-yl)-1,2,3,4,12,12a-hexahydro-6H-benzo[f]pyrazino[2,1-c][1,4]oxazepin-6-one;
1-((12aS)-10-Chloro-9-(5-methyl-1H-indazol-4-yl)-3,4,12,12a-tetrahydro-6H-benzo[f]pyrazino[2,1-c][1,4]oxazepin-2(1H)-yl)prop-2-en-1-one;
1-[(12aR)-10-Chloro-9-(2-fluoro-6-hydroxyphenyl)-7-methoxy-3,4,12,12a-tetrahydro-6H-pyrazino[2,1-c][1,4]benzoxazepin-2(1H)-yl]prop-2-en-1-one;
(12aR)-10-chloro-9-(2-fluoro-6-hydroxyphenyl)-7-hydroxy-2-(prop-2-enoyl)-1,2,3,4,12,12a-hexahydro-6H-pyrazino[2,1-c][1,4]benzoxazepin-6-one;
(12aR)-10-Chloro-9-(5-methyl-1H-indazol-4-yl)-2-(prop-2-enoyl)-1,2,3,4,12,12a-hexahydro-6H-pyrazino[2,1-c][1,4]benzoxazepin-6-one;
1-((12aR)-10-Chloro-9-(5-methyl-1H-indazol-4-yl)-3,4,12,12a-tetrahydro-6H-benzo[f]pyrazino[2,1-c][1,4]oxazepin-2(1H)-yl)prop-2-en-1-one;
(12aR)-10-Chloro-8-fluoro-9-(2-fluoro-6-hydroxyphenyl)-2-(prop-2-enoyl)-1,2,3,4,12,12a-hexahydro-6H-pyrazino[2,1-c][1,4]benzoxazepin-6-one;
1-[(12aR)-8,10-dichloro-9-(2-fluoro-6-hydroxyphenyl)-7-hydroxy-3,4,12,12a-tetrahydro-6H-pyrazino[2,1-c][1,4]benzoxazepin-2(1H)-yl]prop-2-en-1-one;
1-[(12aR)-10-Chloro-9-(2-fluoro-6-hydroxyphenyl)-7-(1H-imidazol-1-yl)-3,4,12,12a-tetrahydro-6H-pyrazino[2,1-c][1,4]benzoxazepin-2(1H)-yl]prop-2-en-1-one;
(12aR)-10-Chloro-9-(2-fluoro-6-hydroxyphenyl)-2-(prop-2-enoyl)-1,2,3,4,12,12a-hexahydro-6H-pyrazino[2,1-c][1,4]benzoxazepine-7-carbonitrile;
1-[(12aR)-10-Chloro-9-(2-fluoro-6-hydroxyphenyl)-7-(1H-pyrazol-1-yl)-3,4,12,12a-tetrahydro-6H-pyrazino[2,1-c][1,4]benzoxazepin-2(1H)-yl]prop-2-en-1-one;
1-((12aR)-10-Chloro-8-fluoro-9-(5-methyl-1H-benzo[d]imidazol-4-yl)-3,4,12,12a-tetrahydro-6H-benzo[f]pyrazino[2,1-c][1,4]oxazepin-2(1H)-yl)prop-2-en-1-one;
(12aR)-8,10-Dichloro-9-(2-fluoro-6-hydroxyphenyl)-2-(prop-2-enoyl)-1,2,3,4,12,12a-hexahydro-6H-pyrazino[2,1-c][1,4]benzoxazepin-6-one;
(12aR)-10-Chloro-9-(2-fluoro-6-hydroxyphenyl)-2-(prop-2-enoyl)-1,2,3,4,12,12a-hexahydro-6H-pyrazino[2,1-c][1,4]benzoxazepine-8-carbonitrile;
(12aR)-10-Chloro-9-(2-fluoro-6-hydroxyphenyl)-8-methyl-2-(prop-2-enoyl)-1,2,3,4,12,12a-hexahydro-6H-pyrazino[2,1-c][1,4]benzoxazepin-6-one;
1-[(12aR)-8,10-Dichloro-9-(2-fluoro-6-hydroxyphenyl)-3,4,12,12a-tetrahydro-6H-pyrazino[2,1-c][1,4]benzoxazepin-2(1H)-yl]prop-2-en-1-one;
8-[(12aR)-10-Chloro-8-fluoro-2-(prop-2-enoyl)-1,2,3,4,12,12a-hexahydro-6H-pyrazino[2,1-c][1,4]benzoxazepin-9-yl]-7-methylisoquinolin-1(2H)-one;
1-[(12aR)-10-chloro-9-(2-fluoro-6-hydroxyphenyl)-8-methoxy-3,4,12,12a-tetrahydro-6H-pyrazino[2,1-c][1,4]benzoxazepin-2(1H)-yl]prop-2-en-1-one;
(12aS)-10-Chloro-9-(5-methyl-1H-indazol-4-yl)-2-(prop-2-enoyl)-1,3,4,11,12,12a-hexahydropyrazino[2,1-c][1,4]benzodiazepin-6(2H)-one;
1-[(12aR)-10-chloro-9-(2-chloro-6-hydroxyphenyl)-8-fluoro-3,4,12,12a-tetrahydro-6H-pyrazino[2,1-c][1,4]benzoxazepin-2(1H)-yl]prop-2-en-1-one;
(12aS)-10-Chloro-11-methyl-9-(5-methyl-1H-indazol-4-yl)-2-(prop-2-enoyl)-1,3,4,11,12,12a-hexahydropyrazino[2,1-c][1,4]benzodiazepin-6(2H)-one;
1-[(12aR)-10-Chloro-9-(2,3-difluoro-6-hydroxyphenyl)-8-fluoro-3,4,12,12a-tetrahydro-6H-pyrazino[2,1-c][1,4]benzoxazepin-2(1H)-yl]prop-2-en-1-one;
(12aR)-10-Chloro-9-(2-hydroxy-6-methylphenyl)-2-(prop-2-enoyl)-1,2,3,4,12,12a-hexahydro-6H-pyrazino[2,1-c][1,4]benzoxazepine-8-carbonitrile;
1-[(12aR)-9-(2-Chloro-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-8,10-Difluoro-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-8,10-Difluoro-9-[2-fluoro-6-(hydroxymethyl)phenyl]-3,4,12,12a-tetrahydro-6H-pyrazino[2,1-c][1,4]benzoxazepin-2(1H)-yl]prop-2-en-1-one;
1-[(12aR)-8,10-Difluoro-9-[2-hydroxy-6-(trifluoromethyl)phenyl]-3,4,12,12a-tetrahydro-6H-pyrazino[2,1-c][1,4]benzoxazepin-2(1H)-yl]prop-2-en-1-one;
1-[(12aR)-9-(2-Ethyl-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-9-[2-(Difluoromethyl)-6-hydroxyphenyl]-8,10-difluoro-3,4,12,12a-tetrahydro-6H-pyrazino[2,1-c][1,4]benzoxazepin-2(1H)-yl]prop-2-en-1-one;
(12aR)-9-(2-Chloro-6-hydroxyphenyl)-10-fluoro-2-(prop-2-enoyl)-1,2,3,4,12,12a-hexahydro-6H-pyrazino[2,1-c][1,4]benzoxazepine-8-carbonitrile;
(12aR)-10-Chloro-9-(2-chloro-6-hydroxyphenyl)-2-(prop-2-enoyl)-1,2,3,4,12,12a-hexahydro-6H-pyrazino[2,1-c][1,4]benzoxazepine-8-carbonitrile;
1-[(12aR)-9-(2-Bromo-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-8-Chloro-10-fluoro-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-8-Chloro-10-ethynyl-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-10-Ethynyl-8-fluoro-9-(2-hydroxy-6-methylphenyl)-3,4,12,12a-tetrahydro-6H-pyrazino[2,1-c][1,4]benzoxazepin-2(1H)-yl]prop-2-en-1-one;
(6aR)-4-Chloro-3-(2-fluoro-6-hydroxyphenyl)-2-methyl-8-(prop-2-enoyl)-2,6,6a,7,8,9,10,12-octahydro-1H-pyrazino[2,1-c]pyrido[3,4-f][1,4]oxazepin-1-one;
1-[(6aR)-1,4-Dichloro-3-(2-fluoro-6-hydroxyphenyl)-6a,7,9,10-tetrahydro-12H-pyrazino[2,1-c]pyrido[3,4-f][1,4]oxazepin-8(6H)-yl]prop-2-en-1-one;
(6aR)-4-Chloro-3-(2-fluoro-6-hydroxyphenyl)-8-(prop-2-enoyl)-2,6,6a,7,8,9,10,12-octahydro-1H-pyrazino[2,1-c]pyrido[3,4-f][1,4]oxazepin-1-one;
1-[(8aR)-6-Chloro-5-(2-fluoro-6-hydroxyphenyl)-8a,9,11,12-tetrahydro-14H-pyrazino[2,1-c][1,2,4]triazolo[4′,3′:1,2]pyrido[3,4-f][1,4]oxazepin-10(8H)-yl]prop-2-en-1-one;
1-[(7aR)-5-chloro-4-(2-fluoro-6-hydroxyphenyl)-1-methyl-1,7a,8,10,11,13-hexahydropyrazino[2′,1′:3,4][1,4]oxazepino[7,6-g]indazol-9(7H)-yl]prop-2-en-1-one; and
1-[(7aR)-5-Chloro-4-(2-fluoro-6-hydroxyphenyl)-2-methyl-2,7a,8,10,11,13-hexahydropyrazino[2′,1′:3,4][1,4]oxazepino[7,6-g]indazol-9(7H)-yl]prop-2-en-1-one;
1-[(12aR)-9-(2-chloro-6-hydroxyphenyl)-8-ethynyl-10-fluoro-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-ethynyl-3,4,12,12a-tetrahydro-6H-pyrazino[2,1-c][1,4]benzoxazepin-2(1H)-yl]prop-2-en-1-one;
1-((12aR)-10-chloro-8-ethynyl-9-(2-fluoro-6-hydroxyphenyl)-3,4,12,12a-tetrahydro-6H-benzo[f]pyrazino[2,1-c][1,4]oxazepin-2(1H)-yl)prop-2-en-1-one;
1-[(7aR)-5-Chloro-4-(2-chloro-6-hydroxyphenyl)-1-methyl-1,7a,8,10,11,13-hexahydroimidazo[4,5-g]pyrazino[2,1-c][1,4]benzoxazepin-9(7H)-yl]prop-2-en-1-one;
1-[(12aR)-8-Chloro-9-(2-chloro-6-hydroxyphenyl)-10-fluoro-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-10-fluoro-8-(prop-1-yn-1-yl)-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-2-ethynyl-6a,7,9,10-tetrahydro-12H-pyrazino[2,1-c]pyrido[2,3-f][1,4]oxazepin-8(6H)-yl]prop-2-en-1-one;
1-[(12aR)-9-(2-Chloro-6-hydroxyphenyl)-8-ethynyl-10-methyl-3,4,12,12a-tetrahydro-6H-pyrazino[2,1-c][1,4]benzoxazepin-2(1H)-yl]prop-2-en-1-one;
1-[(12aR)-10-chloro-9-(2-chloro-6-hydroxyphenyl)-7,8-difluoro-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-(difluoromethoxy)-10-fluoro-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-ethynyl-7,10-difluoro-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-(difluoromethoxy)-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-(cyclopropyloxy)-10-fluoro-3,4,12,12a-tetrahydro-6H-pyrazino[2,1-c][1,4]benzoxazepin-2(1H)-yl]prop-2-en-1-one;
1-((12aR)-9-(2-Chloro-6-hydroxyphenyl)-8-(3-(dimethylamino)prop-1-yn-1-yl)-10-fluoro-3,4,12,12a-tetrahydro-6H-benzo[f]pyrazino[2,1-c][1,4]oxazepin-2(1H)-yl)prop-2-en-1-one;
1-[(12aR)-9-(2-chloro-6-hydroxyphenyl)-10-fluoro-8-[(pyridin-4-yl)methoxy]-3,4,12,12a-tetrahydro-6H-pyrazino[2,1-c][1,4]benzoxazepin-2(1H)-yl]prop-2-en-1-one;
1-[(12aR)-10-chloro-9-(2-chloro-6-hydroxyphenyl)-8-(2-methoxyethoxy)-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-10-fluoro-8-[2-(piperidin-1-yl)ethoxy]-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-(prop-1-yn-1-yl)-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-2-[(²H3)methyloxy]-6a,7,9,10-tetrahydro-12H-pyrazino[2,1-c]pyrido[2,3-f][1,4]oxazepin-8(6H)-yl]prop-2-en-1-one;
1-[(12aR)-10-Chloro-9-(2-chloro-6-hydroxyphenyl)-8-(methoxymethyl)-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-7-[2-(dimethylamino)ethoxy]-10-fluoro-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-1-(prop-1-yn-1-yl)-6a,7,9,10-tetrahydro-12H-pyrazino[2,1-c]pyrido[3,4-f][1,4]oxazepin-8(6H)-yl]prop-2-en-1-one; and
1-((6aR)-4-Chloro-3-(2-chloro-6-hydroxyphenyl)-1-ethynyl-6a,7,9,10-tetrahydro-12H-pyrazino[2,1-c]pyrido[3,4-f][1,4]oxazepin-8(6H)-yl)prop-2-en-1-one;
or a pharmaceutically acceptable salt thereof.

10. The pharmaceutical product according to claim 2, wherein the RASG12C inhibitor is 1-[(12aR)-9-(2-chloro-6-hydroxyphenyl)-8-ethynyl-10-fluoro-3,4,12,12a-tetrahydro-6H-pyrazino[2,1-c][1,4]benzoxazepin-2(1H)-yl]prop-2-en-1-one.

11. The pharmaceutical product according to claim 2, wherein the RASG12C inhibitor is 1-[(12aR)-9-(2-chloro-6-hydroxyphenyl)-10-fluoro-8-(prop-1-yn-1-yl)-3,4,12,12a-tetrahydro-6H-pyrazino[2,1-c][1,4]benzoxazepin-2(1H)-yl]prop-2-en-1-one.

12. The pharmaceutical product according to claim 1, wherein the RASG12C inhibitor is selected from LY3537982 (Loxo/Lilly), AZD4625 (AstraZeneca), sotorasib (AMG510), adagrasib (MRTX849), JDQ443 (Novartis), GDC-6036 (Genentech), BI 1,823,911 (Boehringer Ingelheim), D1553 (InventisBio) and JNJ-74699157 (Johnson and Johnson), or a pharmaceutically acceptable salt thereof.

13. The pharmaceutical product according to claim 1, wherein the RASG12C inhibitor is Compound A represented by the following formula: 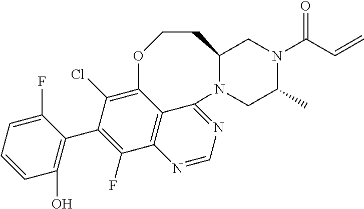 [SMILES: C=CC(=O)N1C[C@@H]2CCOc3c(Cl)c(-c4c(O)cccc4F)c(F)c4ncnc(c34)N2C[C@H]1C]
or a pharmaceutically acceptable salt thereof.

14. The pharmaceutical product according to any one of claims 1 to 13, wherein the anti-HER2 antibody is an antibody comprising a heavy chain comprising CDRH1 consisting of an amino acid sequence represented by SEQ ID NO: 3, CDRH2 consisting of an amino acid sequence represented by SEQ ID NO: 4 and CDRH3 consisting of an amino acid sequence represented by SEQ ID NO: 5, and a light chain comprising CDRL1 consisting of an amino acid sequence represented by SEQ ID NO: 6, CDRL2 consisting of an amino acid sequence consisting of amino acid residues 1 to 3 of SEQ ID NO: 7 and CDRL3 consisting of an amino acid sequence represented by SEQ ID NO: 8.

15. The pharmaceutical product according to any one of claims 1 to 13, wherein the anti-HER2 antibody is an antibody comprising a heavy chain comprising a heavy chain variable region consisting of an amino acid sequence represented by SEQ ID NO: 9 and a light chain comprising a light chain variable region consisting of an amino acid sequence represented by SEQ ID NO: 10.

16. The pharmaceutical product according to any one of claims 1 to 13, wherein the anti-HER2 antibody is an antibody comprising a heavy chain consisting of an amino acid sequence represented by SEQ ID NO: 1 and a light chain consisting of an amino acid sequence represented by SEQ ID NO: 2.

17. The pharmaceutical product according to any one of claims 1 to 13, wherein the anti-HER2 antibody is an antibody comprising a heavy chain consisting of an amino acid sequence represented by SEQ ID NO: 11 and a light chain consisting of an amino acid sequence represented by SEQ ID NO: 2.

18. The pharmaceutical product according to any one of claims 1 to 17, wherein the antibody-drug conjugate is an antibody-drug conjugate in which a drug-linker represented by the following formula: 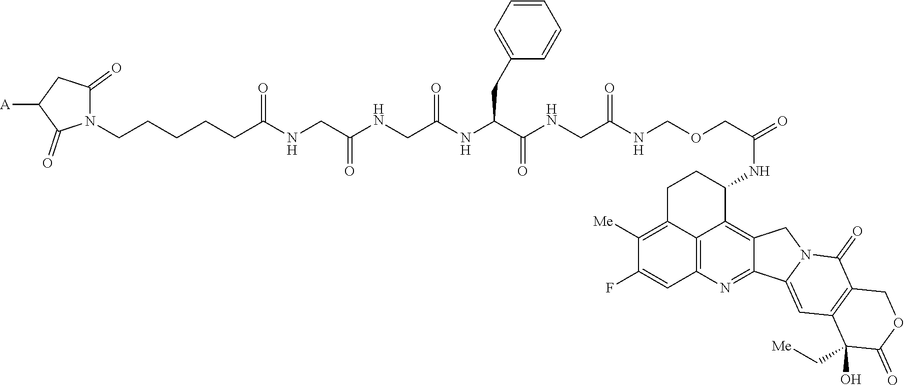
wherein A represents the connecting position to an antibody, is conjugated to an anti-HER2 antibody via a thioether bond.

19. The pharmaceutical product according to any one of claims 1 to 18, wherein the anti-HER2 antibody-drug conjugate is represented by the following formula: 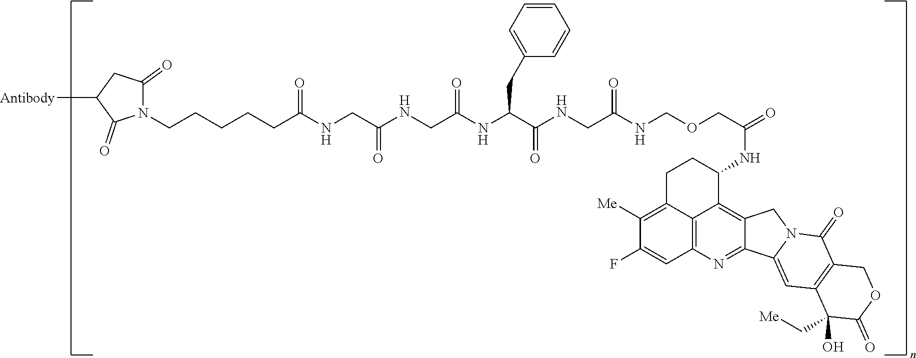
wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8.

20. The pharmaceutical product according to any one of claims 1 to 13, wherein the anti-HER2 antibody-drug conjugate is trastuzumab deruxtecan (DS-8201) or trastuzumab emtansine (T-DM1).

21. The pharmaceutical product according to claim 20, wherein the anti-HER2 antibody-drug conjugate is trastuzumab deruxtecan (DS-8201).

22. The pharmaceutical product according to any one of claims 1 to 21 wherein the product is a composition comprising the anti-HER2 antibody-drug conjugate and the RASG12C inhibitor, for separate simultaneous administration.

23. The pharmaceutical product according to any one of claims 1 to 21 wherein the product is a combined preparation comprising the anti-HER2 antibody-drug conjugate and the RASG12C inhibitor, for sequential administration.

24. The pharmaceutical product according to any one of claims 1 to 23, wherein the product is for treating cancer.

25. The pharmaceutical product according to claim 24, wherein the cancer is at least one selected from the group consisting of breast cancer, gastric cancer, colorectal cancer, lung cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, and melanoma.

26. The pharmaceutical product according to claim 25, wherein the cancer is breast cancer.

27. The pharmaceutical product according to claim 26, wherein the breast cancer has a HER2 status score of IHC 3+.

28. The pharmaceutical product according to claim 26, wherein the breast cancer is HER2 low-expressing breast cancer.

29. The pharmaceutical product according to claim 26, wherein the breast cancer has a HER2 status score of IHC 2+.

30. The pharmaceutical product according to claim 26, wherein the breast cancer has a HER2 status score of IHC 1+.

31. The pharmaceutical product according to claim 26, wherein the breast cancer has a HER2 status score of IHC>0 and <1+.

32. The pharmaceutical product according to claim 26, wherein the breast cancer is triple-negative breast cancer.

33. The pharmaceutical product according to claim 24, wherein the cancer is gastric cancer.

34. The pharmaceutical product according to claim 24, wherein the cancer is colorectal cancer.

35. The pharmaceutical product according to claim 24, wherein the cancer is lung cancer.

36. The pharmaceutical product according to claim 35, wherein the lung cancer is non-small cell lung cancer.

37. The pharmaceutical product according to claim 24, wherein the cancer is pancreatic cancer.

38. The pharmaceutical product according to claim 24, wherein the cancer is ovarian cancer.

39. The pharmaceutical product according to claim 24, wherein the cancer is prostate cancer.

40. The pharmaceutical product according to claim 24, wherein the cancer is kidney cancer.

41. A pharmaceutical product as defined in any one of claims 1 to 23, for use in treating cancer.

42. The pharmaceutical product for the use according to claim 41, wherein the cancer is as defined in any one of claims 25 to 40.

43. Use of an anti-HER2 antibody-drug conjugate in the manufacture of a medicament for use in combination with a RASG12C inhibitor, wherein the anti-HER2 antibody-drug conjugate and the RASG12C inhibitor are as defined in any one of claims 1 to 21, for treating cancer.

44. The use according to claim 43 wherein the medicament is for use in combination with the RASG12C inhibitor by sequential administration.

45. The use according to claim 43 wherein the medicament is for use in combination with the RASG12C inhibitor by separate simultaneous administration.

46. Use of a RASG12C inhibitor in the manufacture of a medicament for use in combination with an anti-HER2 antibody-drug conjugate, wherein the anti-HER2 antibody-drug conjugate and the RASG12C inhibitor are as defined in any one of claims 1 to 21, for treating cancer.

47. The use according to claim 46 wherein the medicament is for use in combination with the anti-HER2 antibody-drug conjugate by sequential administration.

48. The use according to claim 46 wherein the medicament is for use in combination with the anti-HER2 antibody-drug conjugate by separate simultaneous administration.

49. The use according to any one of claims 43 to 48, wherein the cancer is as defined in any one of claims 25 to 40.

50. An anti-HER2 antibody-drug conjugate for use, in combination with a RASG12C inhibitor, in the treatment of cancer, wherein the anti-HER2 antibody-drug conjugate and the RASG12C inhibitor are as defined in any one of claims 1 to 21.

51. The anti-HER2 antibody-drug conjugate for the use according to claim 50, wherein the cancer is as defined in any one of claims 25 to 40.

52. The anti-HER2 antibody-drug conjugate for the use according to claim 50 or 51, wherein the use comprises administration of the anti-HER2 antibody-drug conjugate and the RASG12C inhibitor sequentially.

53. The anti-HER2 antibody-drug conjugate for the use according to claim 50 or 51, wherein the use comprises administration of the anti-HER2 antibody-drug conjugate and the RASG12C inhibitor separately and simultaneously.

54. An anti-HER2 antibody-drug conjugate for use in the treatment of cancer in a subject, wherein said treatment comprises the sequential or separate simultaneous administration of i) said anti-HER2 antibody-drug conjugate, and ii) a RASG12C inhibitor to said subject, wherein said anti-HER2 antibody-drug conjugate and said RASG12C inhibitor are as defined in any one of claims 1 to 21.

55. A RASG12C inhibitor for use, in combination with an anti-HER2 antibody-drug conjugate, in the treatment of cancer, wherein the anti-HER2 antibody-drug conjugate and the RASG12C inhibitor are as defined in any one of claims 1 to 21.

56. The RASG12C inhibitor for the use according to claim 50, wherein the cancer is as defined in any one of claims 25 to 40.

57. The RASG12C inhibitor for the use according to claim 55 or 56, wherein the use comprises administration of the anti-HER2 antibody-drug conjugate and the RASG12C inhibitor sequentially.

58. The RASG12C inhibitor for the use according to claim 55 or 56, wherein the use comprises administration of the anti-HER2 antibody-drug conjugate and the RASG12C inhibitor separately and simultaneously.

59. A RASG12C inhibitor for use in the treatment of cancer in a subject, wherein said treatment comprises the sequential or separate simultaneous administration of i) said RASG12C inhibitor, and ii) an anti-HER2 antibody-drug conjugate to said subject, wherein said RASG12C inhibitor and said anti-HER2 antibody-drug conjugate are as defined in any one of claims 1 to 21.

60. A method of treating cancer comprising administering an anti-HER2 antibody-drug conjugate and a RASG12C inhibitor as defined in any one of claims 1 to 21 in combination to a subject in need thereof.

61. The method according to claim 60, wherein the cancer is as defined in any one of claims 25 to 40.

62. The method according to claim 60 or 61, wherein the method comprises administering the anti-HER2 antibody-drug conjugate and the RASG12C inhibitor sequentially.

63. The method according to claim 60 or 61, wherein the method comprises administering the anti-HER2 antibody-drug conjugate and the RASG12C inhibitor separately and simultaneously.
